SEQUENCE LISTING (276 sequences)

SEQ ID NO: 1 - protein, 120 aa, Artificial Sequence
  source: /note=“Description of Artificial Sequence Synthetic polypeptide”
EVQLVQSGAEVKKPGASVKVSCKASGYTFTGYYMHWVRQAPGQGLEWMGWINPNSGGTNYAQKFQGWVTMTRDTSISTAYMELSRLRSDDTAVYYCARDSTMAPGAFDIWGRGTLVTVSS

SEQ ID NO: 2 - protein, 110 aa, Artificial Sequence
  source: /note=“Description of Artificial Sequence Synthetic polypeptide”
QSVLTQPPSVSVAPGQTARMTCGGNNIESKTVHWYQQKPGQAPVLVVYNDNVRPSGIPARFSGSNSGNTATLTINRVEAGDEADYYCQVWDSSRDQGVFGGGTKLTVLGA

SEQ ID NO: 3 - protein, 118 aa, Artificial Sequence
  source: /note=“Description of Artificial Sequence Synthetic polypeptide”
EVQLVQSGSEVRRPGSSVRVSCTASGDTSSSFTVNWLRQAPGQGLEWMGGITPMFGTANYAQMFEDRVTITADEMELSGLTSEDTAVYFCATGPSDYVWGSYRFLDTWGRGTTVTVSS

SEQ ID NO: 4 - protein, 112 aa, Artificial Sequence
  source: /note=“Description of Artificial Sequence Synthetic polypeptide”
QAVLTQPSSVSAAPGQEVSISCSGARSNVGGNYVSWYQHLPGTAPKLLIYDNNKRPSGMPDRFSGSKSGTSATLGITGVQTEDEADYYCATWDSSLSAVVFGGGTKLTVLGA

SEQ ID NO: 5 - protein, 118 aa, Artificial Sequence
  source: /note=“Description of Artificial Sequence Synthetic polypeptide”
QVQLVQSGSEVRRPGSSVRISCTASGDTSSSFTVNWVRQAPGQGLEWMGGITPMFGTANYAQVFEDRVTIIADEMELSGLTSEDTAVYFCATGPSDYVWGSYRFLDRWGRGTLVTVSS

SEQ ID NO: 6 - protein, 112 aa, Artificial Sequence
  source: /note=“Description of Artificial Sequence Synthetic polypeptide”
QSVLTQPPSVSAAPGQKVTISCSGGRSSIGNNYVSWYQHLPGTAPKLLIYDNNQRPSGIPDRFSGSKSGTSATLGITGLQTGDEADYYCGTWDSSLSAVVFGGGTKVTVLGA

SEQ ID NO: 7 - protein, 117 aa, Artificial Sequence
  source: /note=“Description of Artificial Sequence Synthetic polypeptide”
EVQLVETGGGLVQPGGSLRLSCAASGFTFSSYGMNWVRQAPGKGLEWVSYISSSGNTIFYADSVKGRFTISRDSAKNSVSLQMNSLRDEDTAVYYCASYYSYYYGMDAWGQGTMVTV

SEQ ID NO: 8 - protein, 112 aa, Artificial Sequence
  source: /note=“Description of Artificial Sequence Synthetic polypeptide”
SYVLTQPPSASGTPGQRVTISCSGSSSNIGSNTVNWYQQLPGTAPKLLIYSNNQRPSGVPDRFSGSKSGTSASLAISGLRSEDEADYYCAAWDYSLSGWVFGGGTKVTVLGA

SEQ ID NO: 9 - protein, 122 aa, Artificial Sequence
  source: /note=“Description of Artificial Sequence Synthetic polypeptide”
EVQLVQSGAEVKKPGASVKVSCKASGYSFTAFYIHWVRQAPGQGLEYLGWIDPNTGATKYAQRFQGRVIMTWDTSITTATMELSRLTSDDSAVYYCVRDLREWGYELSVEYWGRGTLVTVSS

SEQ ID NO: 10 - protein, 112 aa, Artificial Sequence
  source: /note=“Description of Artificial Sequence Synthetic polypeptide”
QSVLTQPPSASGTPGQRVTISCSGSSSNIGSNYVYWYQQLPGTAPKLLIYRNNQRPSGVPDRFSGSKSGTSASLAISGLRSEDEADYYCAAWDDSLSGWVFGGGTKLTVLGA

SEQ ID NO: 11 - protein, 115 aa, Artificial Sequence
  source: /note=“Description of Artificial Sequence Synthetic polypeptide”
EVQLVETGGGVVQPGGSLSLSCAASGFTFSSYGMQWVRQAPGKGLEWVAFIRYDGSSEYYADSVKGRFTISRDNSKNTLYLQMNSLRAEDTAVYYCGRTLESSLWGKGTLVTVSS

SEQ ID NO: 12 - protein, 112 aa, Artificial Sequence
  source: /note=“Description of Artificial Sequence Synthetic polypeptide”
QSVLTQPPSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTRTKLTVLGA

SEQ ID NO: 13 - protein, 118 aa, Artificial Sequence
  source: /note=“Description of Artificial Sequence Synthetic polypeptide”
QVQLQESGPGLVKPSQTLSLTCGISGDSVSSNSAAWNWIRQSPTRGLEWLGRTYYRSSWYHNYAPSMNSRLTIIADTSKNQFSLQLNSVTPEDTAVYYCASGWAFDVWGRGTLVTVSS

SEQ ID NO: 14 - protein, 112 aa, Artificial Sequence
  source: /note=“Description of Artificial Sequence Synthetic polypeptide”
QSVLTQPPSASGSPGQSVTISCTGTSSDVGAYDFVSWYQQHPGKAPKLMIYEVNKRPSGVPDRFSGSKSGNTASLTVSGLQAEDEADYYCSSYAGSKNLLFGGGTKLTVLGA

SEQ ID NO: 15 - protein, 119 aa, Artificial Sequence
  source: /note=“Description of Artificial Sequence Synthetic polypeptide”
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARQSGADWYFDLWGRGTLVTVSS

SEQ ID NO: 16 - protein, 113 aa, Artificial Sequence
  source: /note=“Description of Artificial Sequence Synthetic polypeptide”
QAVLTQPSAVSGAPGQRVTISCTGTSSNIGTNYLVHWYQQRPGTAPQLLVSGNNTRPSGVTDRFSVSKSATSASLAITGLQAEDEADYYCQTYDINLRVWVFGGGTKVTVLGA

SEQ ID NO: 17 - protein, 125 aa, Artificial Sequence
  source: /note=“Description of Artificial Sequence Synthetic polypeptide”
QVQLVQSGAEVKKPGSSVKVSCKASGYTFTSYGISWVRQAPGQGLEWMGWISAYNGNTNYAQKLQGRVTMTTDTSTSTAYMELRSLRSDDTAVYYCARVPGVSGSYPDYYYMDVWGKGTLVTVSS

SEQ ID NO: 18 - protein, 109 aa, Artificial Sequence
  source: /note=“Description of Artificial Sequence Synthetic polypeptide”
DIQMTQSPSTLSASIGDRVTITCRASEGIYHWLAWYQQKPGKAPKLLIYKASSLASGAPSRFSGSGSGTDFTLTISSLQPDDFATYYCQQYSNYPLTFGGGTKLEIKRA

SEQ ID NO: 19 - protein, 120 aa, Artificial Sequence
  source: /note=“Description of Artificial Sequence Synthetic polypeptide”
EVQLVQSGGGLVRPGGSLRLSCAASGFSFSDYYMTWIRQIPGKGLEWVAVIWNDGSDRYYADSVKGRFTISRDNSKNTLFLQMSSLRDEDTALYYCVRGGPTASSGFDYWGRGTLVTVSS

SEQ ID NO: 20 - protein, 112 aa, Artificial Sequence
  source: /note=“Description of Artificial Sequence Synthetic polypeptide”
SSELTQPASVSGSPGQSITISCTGTSSDVGGYNYVSWYLQHPGKAPKLMIYEGSKRPSGVSNRFSGSKSGNTASLTISGLQAEDEADYYCSSYTTRSTRVFGGGTKLTVLGA

SEQ ID NO: 21 - protein, 119 aa, Artificial Sequence
  source: /note=“Description of Artificial Sequence Synthetic polypeptide”
EVQLVQSGAEVKKPGESLKISCKGFGYNFRSAWIGWVRQMPGKGLEWMGVIYPGDSDVRYSPSFQGQVTISADKSISTAYLQWSSLKASDTAMYYCTRPVGQWVDSDYWGKGTLVTVSS

SEQ ID NO: 22 - protein, 112 aa, Artificial Sequence
  source: /note=“Description of Artificial Sequence Synthetic polypeptide”
QSVLTQPPSASGTPGQRVTISCSGSSSNIGTNTVNWYQQLPGTAPKLLIYTSNQRPSGVPARFSASNSGTSASLAISGLRSEDEADYYCAAWDDKLSGAVFGGGTKLTVLGA

SEQ ID NO: 23 - protein, 120 aa, Artificial Sequence
  source: /note=“Description of Artificial Sequence Synthetic polypeptide”
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARWRPLLDYHFDQWGQGTMVTVSS

SEQ ID NO: 24 - protein, 112 aa, Artificial Sequence
  source: /note=“Description of Artificial Sequence Synthetic polypeptide”
QSVLTQPPSASGTPGQTVTISCSGSSSNIGSSVVNWYQQFPGTAPKVLVYSNTQRPSGVPDRFSGSRSGTSASLAISGLQSEDEADYYCLAWDASLNGWVFGGGTKLTVLGA

SEQ ID NO: 25 - protein, 119 aa, Artificial Sequence
  source: /note=“Description of Artificial Sequence Synthetic polypeptide”
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGYSGYDDPDSWGRGTTVTVSS

SEQ ID NO: 26 - protein, 112 aa, Artificial Sequence
  source: /note=“Description of Artificial Sequence Synthetic polypeptide”
HVILTQPPSTSGTPGQTVTISCSGSSSNIGSHYVYWYQQLPGTAPKLLIYRNNQRPSGVPDRFSGSKSGTSASLAISGLRSEDETDYYCAAWDDSLSGRVFGTGTKLTVLGA

SEQ ID NO: 27 - protein, 114 aa, Artificial Sequence
  source: /note=“Description of Artificial Sequence Synthetic polypeptide”
QVQLQQSGAEVKKPGSSVKVSCKASGGTISNYAISWVRLAPGQGLEWMGSIVPLHGTTNFAQKFQGRVTITADESTSTSYMEVNVLTYEDTAMYYCASLNWGYWGRGTLVTVSS

SEQ ID NO: 28 - protein, 111 aa, Artificial Sequence
  source: /note=“Description of Artificial Sequence Synthetic polypeptide”
NFMLTQPHSVSESPGKTVTISCTGSSGSIASNYVQWYQQRPDSAPTTVIYEDNRRSSGVPDRFSGSIDSNSASLSISGLKTEDEADYYCQSYDSSGHVVFGGGTKLTVLGA

SEQ ID NO: 29 - protein, 120 aa, Artificial Sequence
  source: /note=“Description of Artificial Sequence Synthetic polypeptide”
EVQLVESGEGLVKPGGSLRLSCTASGFTFRSYSLNWVRQAPGQGLEWVSSISSTSTYIYYADSVKGRFTISRDDAKNTLYLQMNSLRAEDTAAYYCVRLGSGGGYFPDYWGRGTLVTVSS

SEQ ID NO: 30 - protein, 110 aa, Artificial Sequence
  source: /note=“Description of Artificial Sequence Synthetic polypeptide”
SSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGA

SEQ ID NO: 31 - protein, 114 aa, Artificial Sequence
  source: /note=“Description of Artificial Sequence Synthetic polypeptide”
QVQLVQSGGGLVQPGGSLRLSCAASGFTFSTYAMSWARQAPGKGLEWVSSISGDGGRILDADSAKGRFTISRDNSKNTLYLQMNGLRVEDTALYYCARADGNYWGRGTMVTVSS

SEQ ID NO: 32 - protein, 112 aa, Artificial Sequence
  source: /note=“Description of Artificial Sequence Synthetic polypeptide”
QSVLTQPASVSGSPGQSITISCTGTSSDVGGYNYVSWYQQHPGKAPKLMIYEGSKRPSGVSNRFSGSKSGNTASLTISGLQAEDEADYYCSSYTTRSTRVFGGGTKLTVLGA

SEQ ID NO: 33 - protein, 121 aa, Artificial Sequence
  source: /note=“Description of Artificial Sequence Synthetic polypeptide”
QVQLVESGAEVKKPGASVKVSCKASGYTFTSYDINWVRQAPGQRLEWMGWINAGNGNTKYSQKFQGRVTITRDTSASTAYMELRSLRSDDTAVYYCARGRSYGHPYYFDYWGQGTLVTVSS

SEQ ID NO: 34 - protein, 112 aa, Artificial Sequence; identical to SEQ ID NO: 32
  source: /note=“Description of Artificial Sequence Synthetic polypeptide”

SEQ ID NO: 35 - protein, 118 aa, Artificial Sequence
  source: /note=“Description of Artificial Sequence Synthetic polypeptide”
EVQLVQSGGGLVKPGGSLRLSCAASGFTFSSYGMHWVRQAPGKGLEWVAGIFYDGGNKYYADSVKGRFTISRDNSKNTLYLQMNSLRAEDTAVYYCARDRGYYYMDVWGKGTTVTVSS

SEQ ID NO: 36 - protein, 113 aa, Artificial Sequence
  source: /note=“Description of Artificial Sequence Synthetic polypeptide”
QSVLTQPPSVSGAPGQRVTISCTGRSSNIGAGHDVHWYQQLPGTAPKLLIYGDSNRPSGVPDRFSGSRSGTSASLAITGLQAEDEADYYCQSYDSSLRGSVFGGGTKVTVLGA

SEQ ID NO: 37 - protein, 123 aa, Artificial Sequence
  source: /note=“Description of Artificial Sequence Synthetic polypeptide”
KVQLVQSGTEVKKPGESLKISCQGSGYRFSSDWIAWVRQMPGKGLEWMGIVYPGDSDTRYSPSFQGQVTISADKSISTAYLQWSGLKASDTAKYYCARVQQAVGAKGYAMDVWGKGTLVTVSS

SEQ ID NO: 38 - protein, 112 aa, Artificial Sequence
  source: /note=“Description of Artificial Sequence Synthetic polypeptide”
QTVVIQEPSFSVSPGGTVTLTCGLSSGSVSTSYYPSWYRQTPGQAPHTLIHNTKIRSSGVPDRFSGSILGNNAALTITGAQADDESDYYCLLYMGSGIYVFGGGTKLTVLGA

SEQ ID NO: 39 - protein, 122 aa, Artificial Sequence
  source: /note=“Description of Artificial Sequence Synthetic polypeptide”
QVQLQESGAGLVKPSGTLSLTCAVSGGSISSGNWWSWVRQPPGKGLEWIGEISHSGSTNYNPSLKSRVTISVDKSKNQFSLNLSSVTAADTAVYYCARVRGTVGDTRGPDYWGQGTLVTVSS

SEQ ID NO: 40 - protein, 110 aa, Artificial Sequence; identical to SEQ ID NO: 30
  source: /note=“Description of Artificial Sequence Synthetic polypeptide”

SEQ ID NO: 41 - protein, 124 aa, Artificial Sequence
  source: /note=“Description of Artificial Sequence Synthetic polypeptide”
EVQLVQSGAEVKKPGASVRVSCKGSGNTFTGHYIHWVRQAPGQGLEWLGWIDPNTGDIQYSENFKGSVTLTRDPSINSVFMDLIRLTSDDTAMYYCAREGAGLANYYYYGLDVWGRGTMVTVSS

SEQ ID NO: 42 - protein, 111 aa, Artificial Sequence
  source: /note=“Description of Artificial Sequence Synthetic polypeptide”
QTVVLQEPSFSVSPGGTVTLTCGLNFGSVSTAYYPSWYQQTPGQAPRTLIYGTNIRSSGVPDRFSGSIVGNKAALTITGAQTEDESDYYCALYMGSGMLFGGGTKVTVLGA

SEQ ID NO: 43 - protein, 123 aa, Artificial Sequence
  source: /note=“Description of Artificial Sequence Synthetic polypeptide”
EVQLVQSGGGVVQPGRSLRLSCAASGFTFSSYGMHWVRQAPGKGLEWVAVISYDGSIKYYADSVKGRFTISRDNSKNTLYLQMNSLRAEDTAVYYCARTGEYSGYDTSGYSNWGQGTLVTVSS

SEQ ID NO: 44 - protein, 111 aa, Artificial Sequence
  source: /note=“Description of Artificial Sequence Synthetic polypeptide”
QSVLTQPPSASGTPGQRVTISCSGSSSNIGSNTVNWYQRLPGAAPQLLIYNNDQRPSGIPDRFSGSKSGTSGSLVISGLQSEDEADYYCASWDDSLNGRVFGGGTKLTVLG

SEQ ID NO: 45 - protein, 121 aa, Artificial Sequence
  source: /note=“Description of Artificial Sequence Synthetic polypeptide”
GVQLVESGGGLVKPGGSLRLSCAASGFTFSSYNMNWVRQAPGKGLEWVSAISGSGGSTYYADSVTGRFTISRDNSKNTLYLQMNSLRAEDTAVYYCAKDTSGWYGDGMDVWGRGTLVTVSS

SEQ ID NO: 46 - protein, 109 aa, Artificial Sequence; identical to SEQ ID NO: 18
  source: /note=“Description of Artificial Sequence Synthetic polypeptide”

SEQ ID NO: 47 - protein, 124 aa, Artificial Sequence
  source: /note=“Description of Artificial Sequence Synthetic polypeptide”
QMQLVQSGGGVVQPGRSLRLSCAASGFTFSSYGMHWVRQAPGKGLEWVAVISYDGSIKYYADSVKGRFTISRDNSKNTLYLQMNSLRAEDTGVYYCSKDRYSSGWYSSDAFDIWGRGTMVTVSS

SEQ ID NO: 48 - protein, 110 aa, Artificial Sequence
  source: /note=“Description of Artificial Sequence Synthetic polypeptide”
SSELTQDPAVSVALGQTVRITCQGDSLRSYYASWYQQKPGQAPVLVIYGKNNRPSGIPDRFSGSSSGNTASLTITGAQAEDEADYYCHSRDSSGNHVLFGGGTKLTVLGA

SEQ ID NO: 49 - protein, 128 aa, Artificial Sequence
  source: /note=“Description of Artificial Sequence Synthetic polypeptide”
EVQLVQSGAEVKKPGESLKISCKGSGYTFTNHWIAWVRQMPGKGLEWMGIIYPGDSETRYSPSFQGHVTISADKSISTAYLQWSTLKDSDSAMYFCVRQARGWDDGRAGYYYSGMDAWGQGTLVTVSS

SEQ ID NO: 50 - protein, 112 aa, Artificial Sequence
  source: /note=“Description of Artificial Sequence Synthetic polypeptide”
QAVVLQEPSFSVSPGGTVTLTCGLRSGSVSTSHYPSWYQQTPGQAPRTLIYSTNTRSSGVPDRFSGSILGNKAALTITGAQADDESNYYCMLYMGSGMYVFGGGTKVTVLGA

SEQ ID NO: 51 - protein, 120 aa, Artificial Sequence
  source: /note=“Description of Artificial Sequence Synthetic polypeptide”
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARVSGSHFPFFDSWGQGTMVTVSS

SEQ ID NO: 52 - protein, 110 aa, Artificial Sequence
  source: /note=“Description of Artificial Sequence Synthetic polypeptide”
QSVLTQPPSVSVAPGQTARITCGGDKIGHKSVHWYQQKPGQAPVLLVYDDRKRPSGIPERFSGSNSGNTATLTISRVEAGDEAAYHCQVWDRSSDPYVFGTGTKVTVLGA

SEQ ID NO: 53 - protein, 119 aa, Artificial Sequence
  source: /note=“Description of Artificial Sequence Synthetic polypeptide”
QVQLVQSGAEVKKPGASVKVSCQASGYTFSGHYMHLVRQAPGQGLEWMGWIHPTSGGTTYAQKFQGRVVMTRDTSISTAYMELSRLTSDDTAVYYCARMSQNYDAFDIWGQGTMVTVSS

SEQ ID NO: 54 - protein, 111 aa, Artificial Sequence
  source: /note=“Description of Artificial Sequence Synthetic polypeptide”
QAVLTQPSSVSGAPGQRVTISCTGSSSNIGAGYDVNWYQQFPGTAPKIIVYGDRPSGAPDRFSGSKSGTSASLAITGLRAEDEADYYCQSWDSRLSSYVFGTGTKVTVLGA

SEQ ID NO: 55 - protein, 123 aa, Artificial Sequence
  source: /note=“Description of Artificial Sequence Synthetic polypeptide”
QVQLQESGGGVVQPGGSLRLSCAASGFTFSGYGMHWVRQAPGKGLEWVASVRNDGSNTYYTDSVKDRFTISRDNTKNTLYLQMNSLRAEDTAVYYCAKSRRVMYGTSYYFDYWGRGTLVTVSS

SEQ ID NO: 56 - protein, 110 aa, Artificial Sequence; identical to SEQ ID NO: 30
  source: /note=“Description of Artificial Sequence Synthetic polypeptide”

SEQ ID NO: 57 - protein, 126 aa, Artificial Sequence
  source: /note=“Description of Artificial Sequence Synthetic polypeptide”
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARDLGIDPLWSGYYTPLDYWGRGTMVTVSS

SEQ ID NO: 58 - protein, 112 aa, Artificial Sequence
  source: /note=“Description of Artificial Sequence Synthetic polypeptide”
HVILTQPPSASGTPGQRVTISCSGSSSNIGSNSVSWYQQLPGTAPKLLMYTNNQRPSGVPDRFSGSKSGTSASLAISGLQSEDEADYYCATWDASLNTWVFGGGTKVTVLGA

SEQ ID NO: 59 - protein, 116 aa, Artificial Sequence
  source: /note=“Description of Artificial Sequence Synthetic polypeptide”
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGGSGSDYWGQGTMVTVSS

SEQ ID NO: 60 - protein, 110 aa, Artificial Sequence
  source: /note=“Description of Artificial Sequence Synthetic polypeptide”
NFMLTQPHSVSGSPGKTVTISCTRSSGYIDSKYVQWYQQRPGSAPTTVIYEDNRRPSGVPDRFSGSIDSNSASLTISGLETEDEADYYCQSYDDTNVVFGGGTKVTVLGA

SEQ ID NO: 61 - protein, 120 aa, Artificial Sequence
  source: /note=“Description of Artificial Sequence Synthetic polypeptide”
EVQLVQSGAEVKEPGASVKVSCKASGYDFSNYGFSWVRQAPGQGLEWMGWISSYNGYTNYAQRLQGRVTMTTDTSTSTAYMELRSLRSDDTAVYYCARDRGLGNWYFDLWGQGTLVTVSS

SEQ ID NO: 62 - protein, 112 aa, Artificial Sequence; identical to SEQ ID NO: 32
  source: /note=“Description of Artificial Sequence Synthetic polypeptide”

SEQ ID NO: 63 - protein, 108 aa, Artificial Sequence
  source: /note=“Description of Artificial Sequence Synthetic polypeptide”
QSVLTQPPSVSVAPGQTARMTCGGNNIESKTVHWYQQKPGQAPVLVVYNDNVRPSGIPARFSGSNSGNTATLTINRVEAGDEADYYCQVWDSSRDQGVFGGGTKLTVL

SEQ ID NO: 64 - protein, 110 aa, Artificial Sequence
  source: /note=“Description of Artificial Sequence Synthetic polypeptide”
QAVLTQPSSVSAAPGQEVSISCSGARSNVGGNYVSWYQHLPGTAPKLLIYDNNKRPSGMPDRFSGSKSGTSATLGITGVQTEDEADYYCATWDSSLSAVVFGGGTKLTVL

SEQ ID NO: 65 - protein, 118 aa, Artificial Sequence
  source: /note=“Description of Artificial Sequence Synthetic polypeptide”
QVQLVQSGSEVRRPGSSVRISCTASGDTSSSFTVNWVRQAPGQGLEWMGGITPMFGTANYAQVFEDRVTIIADEMELSGLTSEDTAVYFCATGPSDYVWGSYRFLDNWGRGTLVTVSS

SEQ ID NO: 66 - protein, 110 aa, Artificial Sequence
  source: /note=“Description of Artificial Sequence Synthetic polypeptide”
QSVLTQPPSVSAAPGQKVTISCSGGRSSIGNNYVSWYQHLPGTAPKLLIYDNNQRPSGIPDRFSGSKSGTSATLGITGLQTGDEADYYCGTWDSSLSAVVFGGGTKVTVL

SEQ ID NO: 67 - protein, 119 aa, Artificial Sequence
  source: /note=“Description of Artificial Sequence Synthetic polypeptide”
EVQLVETGGGLVQPGGSLRLSCAASGFTFSSYGMNWVRQAPGKGLEWVSYISSSGNTIFYADSVKGRFTISRDSAKNSVSLQMNSLRDEDTAVYYCASYYSYYYGMDAWGQGTMVTVSS

SEQ ID NO: 68 - protein, 110 aa, Artificial Sequence
  source: /note=“Description of Artificial Sequence Synthetic polypeptide”
SYVLTQPPSASGTPGQRVTISCSGSSSNIGSNTVNWYQQLPGTAPKLLIYSNNQRPSGVPDRFSGSKSGTSASLAISGLRSEDEADYYCAAWDYSLSGWVFGGGTKVTVL

SEQ ID NO: 69 - protein, 110 aa, Artificial Sequence
  source: /note=“Description of Artificial Sequence Synthetic polypeptide”
QSVLTQPPSASGTPGQRVTISCSGSSSNIGSNYVYWYQQLPGTAPKLLIYRNNQRPSGVPDRFSGSKSGTSASLAISGLRSEDEADYYCAAWDDSLSGWVFGGGTKLTVL

SEQ ID NO: 70 - protein, 110 aa, Artificial Sequence
  source: /note=“Description of Artificial Sequence Synthetic polypeptide”
QSVLTQPPSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTRTKLTVL

SEQ ID NO: 71 - protein, 110 aa, Artificial Sequence
  source: /note=“Description of Artificial Sequence Synthetic polypeptide”
QSVLTQPPSASGSPGQSVTISCTGTSSDVGAYDFVSWYQQHPGKAPKLMIYEVNKRPSGVPDRFSGSKSGNTASLTVSGLQAEDEADYYCSSYAGSKNLLFGGGTKLTVL

SEQ ID NO: 72 - protein, 111 aa, Artificial Sequence
  source: /note=“Description of Artificial Sequence Synthetic polypeptide”
QAVLTQPSAVSGAPGQRVTISCTGTSSNIGTNYLVHWYQQRPGTAPQLLVSGNNTRPSGVTDRFSVSKSATSASLAITGLQAEDEADYYCQTYDINLRVWVFGGGTKVTVL

SEQ ID NO: 73 - protein, 107 aa, Artificial Sequence
  source: /note=“Description of Artificial Sequence Synthetic polypeptide”
DIQMTQSPSTLSASIGDRVTITCRASEGIYHWLAWYQQKPGKAPKLLIYKASSLASGAPSRFSGSGSGTDFTLTISSLQPDDFATYYCQQYSNYPLTFGGGTKLEIK

SEQ ID NO: 74 - protein, 110 aa, Artificial Sequence
  source: /note=“Description of Artificial Sequence Synthetic polypeptide”
SSELTQPASVSGSPGQSITISCTGTSSDVGGYNYVSWYLQHPGKAPKLMIYEGSKRPSGVSNRFSGSKSGNTASLTISGLQAEDEADYYCSSYTTRSTRVFGGGTKLTVL

SEQ ID NO: 75 - protein, 110 aa, Artificial Sequence
  source: /note=“Description of Artificial Sequence Synthetic polypeptide”
QSVLTQPPSASGTPGQRVTISCSGSSSNIGTNTVNWYQQLPGTAPKLLIYTSNQRPSGVPARFSASNSGTSASLAISGLRSEDEADYYCAAWDDKLSGAVFGGGTKLTVL

SEQ ID NO: 76 - protein, 110 aa, Artificial Sequence
  source: /note=“Description of Artificial Sequence Synthetic polypeptide”
QSVLTQPPSASGTPGQTVTISCSGSSSNIGSSVVNWYQQFPGTAPKVLVYSNTQRPSGVPDRFSGSRSGTSASLAISGLQSEDEADYYCLAWDASLNGWVFGGGTKLTVL

SEQ ID NO: 77 - protein, 110 aa, Artificial Sequence
  source: /note=“Description of Artificial Sequence Synthetic polypeptide”
HVILTQPPSTSGTPGQTVTISCSGSSSNIGSHYVYWYQQLPGTAPKLLIYRNNQRPSGVPDRFSGSKSGTSASLAISGLRSEDETDYYCAAWDDSLSGRVFGTGTKLTVL

SEQ ID NO: 78 - protein, 109 aa, Artificial Sequence
  source: /note=“Description of Artificial Sequence Synthetic polypeptide”
NFMLTQPHSVSESPGKTVTISCTGSSGSIASNYVQWYQQRPDSAPTTVIYEDNRRSSGVPDRFSGSIDSNSASLSISGLKTEDEADYYCQSYDSSGHVVFGGGTKLTVL

SEQ ID NO: 79 - protein, 108 aa, Artificial Sequence
  source: /note=“Description of Artificial Sequence Synthetic polypeptide”
SSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVL

SEQ ID NO: 80 - protein, 110 aa, Artificial Sequence
  source: /note=“Description of Artificial Sequence Synthetic polypeptide”
QSVLTQPASVSGSPGQSITISCTGTSSDVGGYNYVSWYQQHPGKAPKLMIYEGSKRPSGVSNRFSGSKSGNTASLTISGLQAEDEADYYCSSYTTRSTRVFGGGTKLTVL

SEQ ID NO: 81 - protein, 110 aa, Artificial Sequence; identical to SEQ ID NO: 80
  source: /note=“Description of Artificial Sequence Synthetic polypeptide”

SEQ ID NO: 82 - protein, 111 aa, Artificial Sequence
  source: /note=“Description of Artificial Sequence Synthetic polypeptide”
QSVLTQPPSVSGAPGQRVTISCTGRSSNIGAGHDVHWYQQLPGTAPKLLIYGDSNRPSGVPDRFSGSRSGTSASLAITGLQAEDEADYYCQSYDSSLRGSVFGGGTKVTVL

SEQ ID NO: 83 - protein, 110 aa, Artificial Sequence
  source: /note=“Description of Artificial Sequence Synthetic polypeptide”
QTVVIQEPSFSVSPGGTVTLTCGLSSGSVSTSYYPSWYRQTPGQAPHTLIHNTKIRSSGVPDRFSGSILGNNAALTITGAQADDESDYYCLLYMGSGIYVFGGGTKLTVL

SEQ ID NO: 84 - protein, 108 aa, Artificial Sequence; identical to SEQ ID NO: 79
  source: /note=“Description of Artificial Sequence Synthetic polypeptide”

SEQ ID NO: 85 - protein, 109 aa, Artificial Sequence
  source: /note=“Description of Artificial Sequence Synthetic polypeptide”
QTVVLQEPSFSVSPGGTVTLTCGLNFGSVSTAYYPSWYQQTPGQAPRTLIYGTNIRSSGVPDRFSGSIVGNKAALTITGAQTEDESDYYCALYMGSGMLFGGGTKVTVL

SEQ ID NO: 86 - protein, 110 aa, Artificial Sequence
  source: /note=“Description of Artificial Sequence Synthetic polypeptide”
QSVLTQPPSASGTPGQRVTISCSGSSSNIGSNTVNWYQRLPGAAPQLLIYNNDQRPSGIPDRFSGSKSGTSGSLVISGLQSEDEADYYCASWDDSLNGRVFGGGTKLTVL

SEQ ID NO: 87 - protein, 107 aa, Artificial Sequence; identical to SEQ ID NO: 73
  source: /note=“Description of Artificial Sequence Synthetic polypeptide”

SEQ ID NO: 88 - protein, 108 aa, Artificial Sequence
  source: /note=“Description of Artificial Sequence Synthetic polypeptide”
SSELTQDPAVSVALGQTVRITCQGDSLRSYYASWYQQKPGQAPVLVIYGKNNRPSGIPDRFSGSSSGNTASLTITGAQAEDEADYYCHSRDSSGNHVLFGGGTKLTVL

SEQ ID NO: 89 - protein, 110 aa, Artificial Sequence
  source: /note=“Description of Artificial Sequence Synthetic polypeptide”
QAVVLQEPSFSVSPGGTVTLTCGLRSGSVSTSHYPSWYQQTPGQAPRTLIYSTNTRSSGVPDRFSGSILGNKAALTITGAQADDESNYYCMLYMGSGMYVFGGGTKVTVL

SEQ ID NO: 90 - protein, 108 aa, Artificial Sequence
  source: /note=“Description of Artificial Sequence Synthetic polypeptide”
QSVLTQPPSVSVAPGQTARITCGGDKIGHKSVHWYQQKPGQAPVLLVYDDRKRPSGIPERFSGSNSGNTATLTISRVEAGDEAAYHCQVWDRSSDPYVFGTGTKVTVL

SEQ ID NO: 91 - protein, 109 aa, Artificial Sequence
  source: /note=“Description of Artificial Sequence Synthetic polypeptide”
QAVLTQPSSVSGAPGQRVTISCTGSSSNIGAGYDVNWYQQFPGTAPKIIVYGDRPSGAPDRFSGSKSGTSASLAITGLRAEDEADYYCQSWDSRLSSYVFGTGTKVTVL

SEQ ID NO: 92 - protein, 108 aa, Artificial Sequence; identical to SEQ ID NO: 79
  source: /note=“Description of Artificial Sequence Synthetic polypeptide”

SEQ ID NO: 93 - protein, 110 aa, Artificial Sequence
  source: /note=“Description of Artificial Sequence Synthetic polypeptide”
HVILTQPPSASGTPGQRVTISCSGSSSNIGSNSVSWYQQLPGTAPKLLMYTNNQRPSGVPDRFSGSKSGTSASLAISGLQSEDEADYYCATWDASLNTWVFGGGTKVTVL

SEQ ID NO: 94 - protein, 108 aa, Artificial Sequence
  source: /note=“Description of Artificial Sequence Synthetic polypeptide”
NFMLTQPHSVSGSPGKTVTISCTRSSGYIDSKYVQWYQQRPGSAPTTVIYEDNRRPSGVPDRFSGSIDSNSASLTISGLETEDEADYYCQSYDDTNVVFGGGTKVTVL

SEQ ID NO: 95 - protein, 110 aa, Artificial Sequence; identical to SEQ ID NO: 80
  source: /note=“Description of Artificial Sequence Synthetic polypeptide”

SEQ ID NO: 96 - DNA, 360 bp, Artificial Sequence
  source: /note=“Description of Artificial Sequence Synthetic polynucleotide”
gaggtccagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatgcacagaagtttcagggctgggtcaccatgaccagggacacgtccatcagcacagcctacatggagctgagcaggctgagatctgacgacacggccgtgtattactgtgcgagagattctactatggccccaggtgcttttgatatctggggccgaggcaccctggtcaccgtctcgagt

SEQ ID NO: 97 - DNA, 321 bp, Artificial Sequence
  source: /note=“Description of Artificial Sequence Synthetic polynucleotide”
cagtctgtgctgactcagccaccctcggtgtcagtggccccaggacagacggccaggatgacctgtgggggaaacaacattgaaagtaaaactgtgcattggtaccagcagaagccgggccaggcccctgtgctggtcgtctacaatgataacgtccggccctcagggatccctgcgcgattctctggctccaactccggcaacacggccaccctgaccatcaacagggtcgaagccggggatgaggccgactattattgtcaggtgtgggactccagtagagatcaaggggtattcggcggagggaccaagctgaccgtc

SEQ ID NO: 98 - DNA, 320 bp, Artificial Sequence
  source: /note=“Description of Artificial Sequence Synthetic polynucleotide”
ggaggcctgggtcctcggtgagggtctcctgcacggcttctggagacacctccagcagctttaccgtcaactggctgcgacaggcccctggacaaggtcttgagtggatgggagggatcacccctatgtttggcactgcaaactacgcacagatgttcgaggacagagtcacgataaccgcggacgaaatggaactgagtggcctgacatctgaggacacggccgtgtatttttgtgcgacaggcccctccgattacgtttgggggagttatcgtttccttgacacctgggggcgggggaccacggtcaccgtctcgagt

SEQ ID NO: 99 - DNA, 330 bp, Artificial Sequence
  source: /note=“Description of Artificial Sequence Synthetic polynucleotide”
caggctgtgctgactcagccgtcctcagtgtctgcggccccaggacaggaggtctccatctcctgctctggagccagatccaacgttgggggtaattatgtttcctggtaccaacacctcccaggaacagcccccaaactcctcatttatgacaataataagcgaccctcagggatgcctgaccgattctctggctccaagtctggcacgtcagccaccctgggcatcaccggagtccagactgaggacgaggccgattattactgcgcaacatgggatagcagcctgagcgctgtggtcttcggcggagggaccaagctgaccgtccta

SEQ ID NO: 100 - DNA, 354 bp, Artificial Sequence
  source: /note=“Description of Artificial Sequence Synthetic polynucleotide”
caggtgcagctggtgcagtctgggtctgaggtgaggaggcctgggtcctcggtgaggatctcctgcacggcttctggagacacctccagcagctttaccgtcaactgggtgcgacaggcccctggacaaggtcttgagtggatgggagggatcacccctatgtttggcactgcaaactacgcacaggtgttcgaggacagagtcacaataatcgcggacgagatggaactgagtggcctgacatctgaggacacggccgtgtatttctgtgcgacaggcccctccgattacgtttgggggagttatcgtttccttgacaactggggcaggggcaccctggtcaccgtctcgagt

SEQ ID NO: 101 - DNA, 330 bp, Artificial Sequence
  source: /note=“Description of Artificial Sequence Synthetic polynucleotide”
cagtctgtgctgactcagccaccctcagtgtctgcggccccagggcagaaggtcaccatctcctgctctggaggcaggtccagcattgggaataattatgtgtcctggtatcaacacctcccaggaacagcccccaaactcctcatctatgacaataatcagcgaccctcagggattcctgaccgattctctggctccaagtctggcacgtcagccaccctgggcatcaccggactccagactggggacgaggccgattattactgcggaacatgggatagcagcctgagtgctgtggtgtttggcggagggaccaaggtcaccgtccta

SEQ ID NO: 102 - DNA, 363 bp, Artificial Sequence
  source: /note=“Description of Artificial Sequence Synthetic polynucleotide”
gaggtgcagctggtggagactgggggaggcttggtacagcctggggggtccctgagactctcctgtgcagcctctggattcaccttcagtagctatggcatgaactgggtccgccaggctccagggaaggggctggagtgggtttcatacattagtagttctggtaataccatattctacgcagactctgtgaagggccgattcaccatctccagagacagtgccaagaattcagtgtctctgcagatgaacagcctgagagacgaggacacggctgtgtattactgtgcttcctactactcctactactacggtatggacgcctggggccaggggacaatggtcaccgtctcgagttcgagt

SEQ ID NO: 103 - DNA, 300 bp, Artificial Sequence
  source: /note=“Description of Artificial Sequence Synthetic polynucleotide”
tctgggacccccgggcagagggtcaccatctcttgttctggaagcagctccaacatcggaagtaatactgtaaactggtaccagcagctcccaggaacggcccccaaactcctcatctatagtaataatcagcggccctcaggggtccctgaccgattctctggctccaagtctggcacctcagcctccctggccatcagtgggctgcggtccgaggatgaggctgattattactgtgcagcatgggattacagcctgagtggttgggtgttcggcggagggaccaaggtcaccgtccta

SEQ ID NO: 104 - DNA, 366 bp, Artificial Sequence
  source: /note=“Description of Artificial Sequence Synthetic polynucleotide”
gaagtgcagctggtgcagtctggggctgaggtgaagaagcctggggcctcagtgaaggtctcctgcaaggcttctgggtacagcttcaccgccttctatattcactgggtgcgacaggcccctggacaaggccttgagtatttgggatggatcgaccctaatactggtgccacaaaatatgcacagcgctttcagggcagggtcatcatgacctgggacacgtccatcaccacagccaccatggaactgagcaggctgacgtctgacgactcggccgtctactactgtgtgagagatttgcgggagtggggctacgaattgtccgttgagtattggggcagaggaaccctggtcaccgtctcgagt

SEQ ID NO: 105 - DNA, 330 bp, Artificial Sequence
  source: /note=“Description of Artificial Sequence Synthetic polynucleotide”
cagtctgtgctgactcagccaccctcagcgtctgggacccccgggcagagggtcaccatctcttgttctggaagcagctccaacatcggaagtaattatgtatactggtaccagcagctcccaggaacggcccccaaactcctcatctataggaataatcagcggccctcaggggtccctgaccgattctctggctccaagtctggcacctcagcctccctggccatcagtgggctccggtccgaggatgaggctgattattactgtgcagcatgggatgacagcctgagtggttgggtgttcggcggagggaccaagctgaccgtccta

SEQ ID NO: 106 - DNA, 345 bp, Artificial Sequence
  source: /note=“Description of Artificial Sequence Synthetic polynucleotide”
gaggtgcagctggtggagactgggggaggcgtggtccagcctggggggtccctgagcctctcctgtgcagcgtctggattcaccttcagtagctatggcatgcagtgggtccgccaggctccaggcaaggggctggagtgggtggcgtttatacggtacgatggaagtagtgaatactatgcagactccgtgaagggccgattcaccatctccagagacaattccaagaacacgctgtatctgcaaatgaacagcctgagagctgaggacacggctgtgtattactgtggaagaacgctggagtctagtttgtggggcaagggaaccctggtcaccgtctcgagt

SEQ ID NO: 107 - DNA, 330 bp, Artificial Sequence
  source: /note=“Description of Artificial Sequence Synthetic polynucleotide”
cagtctgtgttgacgcagccgccctcagtgtctgcggccccaggacagaaggtcaccatttcctgctctggaagcacctccaacattgggaataattatgtctcctggtaccaacagcacccaggcaaagcccccaaactcatgatttatgatgtcagtaagcggccctcaggggtccctgaccgattctctggctccaagtctggcaactcagcctccctggacatcagtgggctccagtctgaggatgaggctgattattactgtgcagcatgggatgacagcctgagtgaatttctcttcggaactaggaccaagctgaccgtccta

SEQ ID NO: 108 - DNA, 354 bp, Artificial Sequence
  source: /note=“Description of Artificial Sequence Synthetic polynucleotide”
caggtgcagctgcaggagtcgggtccaggactggtgaagccctcgcagaccttgtcactcacctgtggcatctccggggacagtgtctctagcaacagtgctgcttggaactggatcaggcagtccccaacgagaggccttgagtggctgggaaggacatattacaggtccagttggtatcataactatgcaccttctatgaacagtcgattaaccatcatcgcagacacatccaaaaaccagttctctttgcaactgaactctgtgactcccgaggacacggctgtatattactgtgcaagcgggtgggcctttgatgtctggggcaggggaaccctggtcaccgtctcgagt

SEQ ID NO: 109 - DNA, 330 bp, Artificial Sequence
  source: /note=“Description of Artificial Sequence Synthetic polynucleotide”
cagtctgtgctgactcagccaccctccgcgtccgggtctcctggacagtcagtcaccatctcctgcactggaaccagcagtgacgttggtgcttatgactttgtctcctggtaccaacagcaccctggcaaagcccccaaactcatgatttatgaggtcaataagcggccctcaggggtccctgatcgcttctctggctccaagtctggcaacacggcctccctgaccgtctctgggctccaggctgaggatgaggctgattattactgcagctcatatgcaggcagcaagaatttgcttttcggcggagggaccaagctgaccgtccta

SEQ ID NO: 110 - DNA, 357 bp, Artificial Sequence
  source: /note=“Description of Artificial Sequence Synthetic polynucleotide”
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgagacagtcgggcgcggactggtacttcgatctctggggccgaggcaccctggtcaccgtctcgagt

SEQ ID NO: 111 - DNA, 333 bp, Artificial Sequence
  source: /note=“Description of Artificial Sequence Synthetic polynucleotide”
caggctgtgctgactcagccgtccgcagtttctggggccccagggcagagggtcaccatctcctgcactgggaccagctccaacatcgggacaaactatcttgtacactggtatcagcaacgtccaggaacagccccccaactcctcgtctctggtaacaacactcgaccctctggggtcactgaccggttctctgtctccaagtctgccacttcagcctccctggccatcactgggctccaggctgaggatgaggctgattattactgccagacctatgacatcaacttgagggtttgggtgttcggcggagggaccaaggtcaccgtccta

SEQ ID NO: 112 - DNA, 375 bp, Artificial Sequence
  source: /note=“Description of Artificial Sequence Synthetic polynucleotide”
caggtgcagctggtgcagtctggagctgaggtgaagaagcctgggtcctcggtgaaggtctcctgcaaggcttctggttacacctttaccagctatggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggccgtgtattactgtgcgagagtcccgggcgtaagtgggagctatccagactactactacatggacgtctggggcaagggaaccctggtcaccgtctcctca

SEQ ID NO: 113 - DNA, 321 bp, Artificial Sequence
  source: /note=“Description of Artificial Sequence Synthetic polynucleotide”
gacatccagatgacccagtctccttccaccctgtctgcatctattggagacagagtcaccatcacctgccgggccagtgagggtatttatcactggttggcctggtatcagcagaagccagggaaagctcctaaactcctgatctataaggcctctagtttagccagtggggccccatcaaggttcagcggcagtggatctgggacagatttcactctcaccatcagcagcctgcagcctgatgattttgcaacttattactgccaacaatatagtaattatccgctcactttcggcggagggaccaagctggagatcaaa

SEQ ID NO: 114 - DNA, 359 bp, Artificial Sequence
  source: /note=“Description of Artificial Sequence Synthetic polynucleotide”
gaggtgcagctggtgcagtctgggggaggcttggtcaggcctggagggtccctgagactctcctgtgcagcctcgggattctccttcagtgactactacatgacctggatccgccagattccagggaaggggctggagtgggtggcagttatatggaatgatggaagtgatagatactatgcagactccgtgaagggccgattcaccatttccagagacaattccaagaacacgctgtttctgcaaatgagcagcctgagagacgaggacacggctctatattactgtgtgagagggggaccaacagcttcaagcggatttgactactggggccgaggcaccctggtcaccgtctcgag

SEQ ID NO: 115 - DNA, 330 bp, Artificial Sequence
  source: /note=“Description of Artificial Sequence Synthetic polynucleotide”
tcgtctgagctgactcagcctgcctccgtgtctgggtctcctggacagtcgatcaccatctcctgcactggaaccagcagtgacgttggtggttataactatgtctcctggtacctacaacacccaggcaaagcccccaaactcatgatttatgagggcagtaagcggccctcaggggtttctaatcgcttctctggctccaagtctggcaacacggcctccctgacaatctctgggctccaggctgaggacgaggctgattattactgcagctcatatacaaccaggagcactcgagttttcggcggagggaccaagctgaccgtccta

SEQ ID NO: 116 - DNA, 357 bp, Artificial Sequence
  source: /note=“Description of Artificial Sequence Synthetic polynucleotide”
gaggtgcagctggtgcagtctggggcagaggtgaaaaagcccggggagtctctgaagatctcctgtaagggttttggatacaattttcgcagcgcctggatcggctgggtgcgccagatgcccggcaaaggcctggagtggatgggggtcatctatcctggtgactctgatgtcagatacagtccgtccttccaaggccaggtcaccatctcagccgacaagtccatcagtaccgcctacctgcagtggagcagcctgaaagcctcggacaccgccatgtattattgtacgagacccgtagggcagtgggtggactctgactattggggcaagggaaccctggtcaccgtctcgagt

SEQ ID NO: 117 - DNA, 330 bp, Artificial Sequence
  source: /note=“Description of Artificial Sequence Synthetic polynucleotide”
cagtctgtgttgacgcagccgccctcagcgtctgggacccccggacagagggtcaccatctcttgttctggaagcagctccaacatcggaactaatactgtgaactggtaccagcagcttccaggaacggcccccaaactcctcatctatactagtaatcagcggccctcaggggtccctgcccgcttctctgcctccaactctggcacctcagcctccctggccatcagtgggctccggtccgaggatgaggctgattattattgtgcagcgtgggatgacaagttgagtggtgcggtgttcggcggagggaccaagctgaccgtccta

SEQ ID NO: 118 - DNA, 360 bp, Artificial Sequence
  source: /note=“Description of Artificial Sequence Synthetic polynucleotide”
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgagatggaggcctcttctagactaccactttgaccaatggggccaagggacaatggtcaccgtctcgagt

SEQ ID NO: 119 - DNA, 330 bp, Artificial Sequence
  source: /note=“Description of Artificial Sequence Synthetic polynucleotide”
cagtctgtgctgactcagccaccctcagcgtctgggacccccggacagacggtaacaatctcttgttctggaagcagctccaacatcggaagtagtgttgttaattggtaccagcagttcccaggaacggcccccaaagtcctcgtctatagtaacactcagcggccctcaggggtccctgaccgattctctggctccaggtctggcacctcagcctccctggccatcagtgggctccagtctgaggatgaggctgattattactgtttagcatgggatgccagcctgaatggttgggtgttcggcggagggaccaagctgaccgtccta

SEQ ID NO: 120 - DNA, 357 bp, Artificial Sequence
  source: /note=“Description of Artificial Sequence Synthetic polynucleotide”
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgagaggatacagtggctacgatgaccctgactcctgggggagagggaccacggtcaccgtctcgagt

SEQ ID NO: 121 - DNA, 330 bp, Artificial Sequence
  source: /note=“Description of Artificial Sequence Synthetic polynucleotide”
cacgttatactgactcaaccgccctcaacgtctgggacccccgggcagacggtcaccatctcttgttctgggagcagctccaacatcggaagtcattatgtatactggtaccagcagctcccaggaacggcccccaaactcctcatctataggaataatcagcggccctcaggggtccctgaccgattctctggctccaagtctggcacctcagcctccctggccatcagtgggctccggtccgaggatgagactgattattactgtgcagcatgggatgacagcctgagtggtcgagtcttcggaactgggaccaagctgaccgtccta

SEQ ID NO: 122 - DNA, 342 bp, Artificial Sequence
  source: /note=“Description of Artificial Sequence Synthetic polynucleotide”
caggtacagctgcagcagtcaggggctgaggtgaagaagcctgggtcctcggtgaaggtctcctgcaaggcttctggaggcaccatcagcaactatgctatcagttgggtgcggctggcccctggacaaggtcttgagtggatgggaagtatcgtccctcttcatgggacaacaaacttcgcacagaaattccagggcagagtcacgatcaccgcggacgagtccacgagcacatcctacatggaggtgaacgtcctgacatatgaagacacggcgatgtattattgtgcgtctctcaattggggctactggggccggggcaccctggtcaccgtctcgagt

SEQ ID NO: 123 - DNA, 333 bp, Artificial Sequence
  source: /note=“Description of Artificial Sequence Synthetic polynucleotide”
aattttatgctgactcagccccactctgtgtcggagtctccggggaagacggtaaccatctcctgcaccggcagtagtggcagcattgccagcaactatgtgcagtggtaccagcagcgcccggacagtgcccccaccactgtgatctatgaggataatcgaagatcctctggagtccctgatcggttctctggctccatcgacagctcctccaactctgcctccctcagcatctctggactgaagactgaggacgaggctgactactactgtcagtcctatgatagtagcggtcatgtggtcttcggcggagggaccaagctgaccgtccta

SEQ ID NO: 124 - DNA, 360 bp, Artificial Sequence
  source: /note=“Description of Artificial Sequence Synthetic polynucleotide”
gaggtgcagctggtggagtctggggaaggcctggtcaagcctggggggtccctgagactctcctgtacagcctctggattcaccttcaggagttatagcttgaactgggtccgccaggctccagggcaggggctggagtgggtctcatccattagtagtactagtacttacatatactacgcagactcggtgaagggccgattcaccatctccagagacgacgccaagaacacactgtatctgcaaatgaacagcctgagagccgaagacacagctgcatattactgtgttagactgggatctggtgggggatattttcctgactactggggcaggggcaccctggtcaccgtctcgagt

SEQ ID NO: 125 - DNA, 324 bp, Artificial Sequence
  source: /note=“Description of Artificial Sequence Synthetic polynucleotide”
tcgtctgagctgactcaggaccctgctgtgtctgtggccttgggacagacagtcaggatcacatgccaaggagacagcctcagaagctattatgcaagctggtaccagcagaagccaggacaggcccctgtacttgtcatctatggtaaaaacaaccggccctcagggatcccagaccgattctctggctccagctcaggaaacacagcttccttgaccatcactggggctcaggcggaagatgaggctgactattactgtaactcccgggacagcagtggtaaccatgtggtattcggcggagggaccaagctgaccgtccta

SEQ ID NO: 126 - DNA, 342 bp, Artificial Sequence
  source: /note=“Description of Artificial Sequence Synthetic polynucleotide”
caggtgcagctggtgcagtctgggggaggcttggtccagccgggggggtccctgagactctcctgtgcagcctctggattcacgtttagtacctatgccatgagttgggcccgccaggctccagggaaggggctggagtgggtctcaagtattagtggtgatggtggaagaattctcgatgcagactccgcgaagggccggttcaccatctccagagacaattccaagaacacgctgtatctgcaaatgaacggcctgagagtcgaggacacggccctttattactgtgcgagagcggacggtaactactggggcagggggacaatggtcaccgtctcttca

SEQ ID NO: 127 - DNA, 330 bp, Artificial Sequence
  source: /note=“Description of Artificial Sequence Synthetic polynucleotide”
cagtctgtgctgactcagcctgcctccgtgtctgggtctcctggacagtcgatcaccatctcctgcactggaaccagcagtgacgttggtggttataactatgtctcctggtaccaacaacacccaggcaaagcccccaaactcatgatttatgagggcagtaagcggccctcaggggtttctaatcgcttctctggctccaagtctggcaacacggcctccctgacaatctctgggctccaggctgaggacgaggctgattattactgcagctcatatacaaccaggagcactcgagttttcggcggagggaccaagctgaccgtccta

SEQ ID NO: 128 - DNA, 363 bp, Artificial Sequence
  source: /note=“Description of Artificial Sequence Synthetic polynucleotide”
caggtgcagctggtggagtctggggctgaggtgaagaagcctggggcctcagtgaaggtctcctgcaaggcttctggatacaccttcaccagttatgatatcaactgggtgcgacaggcccccggacaaaggcttgagtggatgggatggatcaacgctggcaatggtaacacaaaatattcacagaagttccagggcagagtcaccattaccagggacacatccgcgagcacagcctacatggagctgaggagcctgagatctgacgacacggccgtgtattactgtgcgagagggaggagctatggccacccgtactactttgactactggggccagggaaccctggtcaccgtctcgagt

SEQ ID NO: 129 - DNA, 330 bp, Artificial Sequence; identical to SEQ ID NO: 127
  source: /note=“Description of Artificial Sequence Synthetic polynucleotide”

SEQ ID NO: 130 - DNA, 354 bp, Artificial Sequence
  source: /note=“Description of Artificial Sequence Synthetic polynucleotide”
gaggtgcagctggtgcagtctgggggaggcctggtcaagcctggggggtccctgagactctcctgtgcagcgtctggattcaccttcagtagctatgggatgcactgggtccgccaggctccaggcaaggggctggagtgggtggcaggtattttttatgatggaggtaataaatactatgcagactccgtgaagggccgattcaccatctccagagacaattccaagaacacgctgtatctgcaaatgaacagcctgagagctgaggacacggctgtgtattactgtgcgagagataggggctactactacatggacgtctggggcaaagggaccacggtcaccgtctcctca

SEQ ID NO: 131 - DNA, 333 bp, Artificial Sequence
  source: /note=“Description of Artificial Sequence Synthetic polynucleotide”
cagtctgtgttgacgcagccgccctcagtgtctggggccccaggacagagggtcaccatctcctgcactgggagaagctccaacatcggggcgggtcatgatgtacactggtaccagcaacttccaggaacagcccccaaactcctcatctatggtgacagcaatcggccctcaggggtccctgaccgattctctggctccaggtctggcacctcagcctccctggccatcactgggctccaggctgaagatgaggctgattattactgccagtcctatgacagcagcctgaggggttcggtattcggcggagggaccaaggtcaccgtccta

SEQ ID NO: 132 - DNA, 369 bp, Artificial Sequence
  source: /note=“Description of Artificial Sequence Synthetic polynucleotide”
aaggtgcagctggtgcagtctgggacagaggtgaaaaagcccggggagtctctgaagatctcctgtcagggttctggatacaggtttagtagtgactggattgcctgggtgcgccagatgcccgggaaaggcctggagtggatggggattgtctatcctggtgactctgataccagatatagcccgtccttccaaggccaagtcaccatctcagccgacaagtccatcagtactgcctacctgcagtggagcggcctgaaggcctcggacaccgccaagtattactgtgcgagagtgcaacaggcagtgggagctaaaggttatgctatggacgtctggggcaagggaaccctggtcaccgtctcgagt

SEQ ID NO: 133 - DNA, 330 bp, Artificial Sequence
  source: /note=“Description of Artificial Sequence Synthetic polynucleotide”
cagactgtggtgatccaggagccatcgttctcagtgtcccctggagggacagtcacactcacttgtggcttgagctctggctcagtctctaccagttactaccccagctggtaccggcagaccccaggccaggctccacacacactcattcacaacacaaagattcgctcctctggggtccctgatcgcttctctggctccatccttgggaacaatgctgccctcaccatcacgggggcccaggcagatgatgaatctgattattactgtcttttgtatatgggtagcggcatttacgtgttcggcggagggaccaagctgaccgtccta

SEQ ID NO: 134 - DNA, 366 bp, Artificial Sequence
  source: /note=“Description of Artificial Sequence Synthetic polynucleotide”
caggtgcagctgcaggagtcgggcgcaggactggtgaagccttcggggaccctgtccctcacctgcgctgtctctggtggctccatcagcagtggtaactggtggagttgggtccgccagcccccagggaaggggctggagtggattggggaaatctctcatagtgggagcaccaactacaacccgtccctcaagagtcgagtcaccatatcagtagacaagtccaagaaccagttctccctgaacctgagttctgtgaccgccgcagacacggccgtgtattactgtgcgagagtaaggggtacggtgggggatacacggggacctgactactggggccagggaaccctggtcaccgtctcgagt

SEQ ID NO: 135 - DNA, 324 bp, Artificial Sequence; identical to SEQ ID NO: 125
  source: /note=“Description of Artificial Sequence Synthetic polynucleotide”

SEQ ID NO: 136 - DNA, 372 bp, Artificial Sequence
  source: /note=“Description of Artificial Sequence Synthetic polynucleotide”
gaagtgcagctggtgcagtctggggctgaggtgaagaagcctggggcctcagtgagggtctcctgcaagggttctggaaacaccttcaccggccactacatccactgggtgcgacaggcccctggacaaggacttgagtggctgggatggatcgaccctaacactggtgacatacagtattcagaaaactttaagggctcggtcaccttgaccagggacccatccatcaactcagtcttcatggacctgatcaggctgacatctgacgacacggccatgtattactgtgcgagagaaggtgccgggctcgccaactactattactacggtctggacgtctggggccgagggacaatggtcaccgtctcgagt

SEQ ID NO: 137 - DNA, 327 bp, Artificial Sequence
  source: /note=“Description of Artificial Sequence Synthetic polynucleotide”
cagactgtggtgctccaggagccttcgttctcagtgtcccctggggggacagtcacactcacttgtggcttgaactttggctcagtctctactgcttactaccccagttggtaccagcagaccccaggccaagctccacgcacgctcatctacggcacaaatattcgttcctctggggtcccggatcgcttctctggctccatcgtagggaacaaagctgccctcaccatcacgggggcccagacagaagatgagtctgattattattgtgcgctgtatatgggtagtggcatgctcttcggcggcgggaccaaggtcaccgtccta

SEQ ID NO: 138 - DNA, 369 bp, Artificial Sequence
  source: /note=“Description of Artificial Sequence Synthetic polynucleotide”
gaggtgcagctggtgcagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtattaaatactatgcagactccgtgaagggccgattcaccatctccagagacaattccaagaacacgctgtatctgcaaatgaacagcctgagagctgaggacacggctgtgtattactgtgcgcgaactggtgaatatagtggctacgatacgagtggttacagcaattggggccaaggcaccctggtcaccgtctcgagt

SEQ ID NO: 139 - DNA, 330 bp, Artificial Sequence
  source: /note=“Description of Artificial Sequence Synthetic polynucleotide”
cagtctgtgctgactcagccaccctcagcgtctgggacccccgggcagagggtcaccatctcttgttctggaagcagctccaacatcgggagtaacactgtaaactggtaccagcgactcccaggagcggccccccaactcctcatctacaataatgaccagcggccctcagggatccctgaccgattctctggctccaagtctggcacctcaggctccctggtcatcagtgggctccagtctgaagatgaggctgattactactgtgcgtcatgggatgacagtctgaatggtcgggtgttcggcggagggaccaagctgaccgtccta

SEQ ID NO: 140 - DNA, 363 bp, Artificial Sequence
  source: /note=“Description of Artificial Sequence Synthetic polynucleotide”
ggggtgcagctggtggagtctgggggaggcctggtcaagcctggggggtccctgagactctcctgtgcagcctctggattcaccttcagtagctataacatgaactgggtccgccaggctccagggaagggactggagtgggtctcagctattagtggtagtggtggtagcacatactacgcagactccgtgacgggccggttcaccatctccagagacaattccaagaacacgctgtatctgcaaatgaacagcctgagagccgaggacacggccgtatattactgtgcgaaagataccagtggctggtacggggacggtatggacgtctggggccggggaaccctggtcaccgtctcgagt

SEQ ID NO: 141 - DNA, 321 bp, Artificial Sequence
  source: /note=“Description of Artificial Sequence Synthetic polynucleotide”
gacatccagatgacccagtctccttccaccctgtctgcatctattggagacagagtcaccatcacctgccgggccagtgagggtatttatcactggttggcctggtatcagcagaagccagggaaagcccctaaactcctgatctataaggcctctagtttagccagtggggccccatcaaggttcagcggcagtggatcagggacagatttcactctcaccatcagcagcctgcagcctgatgattttgcaacttattactgccaacaatatagtaattatccgctcactttcggcggagggaccaagctggagatcaaa

SEQ ID NO: 142 - DNA, 372 bp, Artificial Sequence
  source: /note=“Description of Artificial Sequence Synthetic polynucleotide”
cagatgcagctggtgcagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtattaaatactatgcagactccgtgaagggccgattcaccatctccagagacaattccaagaacacactgtatctacaaatgaacagcctgagagccgaggacacgggcgtttattactgttcgaaagatcgctatagcagtggctggtacagctccgatgcttttgatatttggggccgagggacaatggtcaccgtctcgagt

SEQ ID NO: 143 - DNA, 321 bp, Artificial Sequence
  source: /note=“Description of Artificial Sequence Synthetic polynucleotide”
tctgagctgactcaggaccctgctgtgtctgtggccttgggacagacagtcaggatcacatgccaaggagacagcctcagaagctattatgcaagctggtaccagcagaagccaggacaggcccctgtacttgtcatctatggtaaaaacaaccggccctcagggatcccagaccgattctctggctccagctcaggaaacacagcttccttgaccatcactggggctcaggcggaagatgaggctgactattactgtcattcccgggacagcagtggtaaccatgtgcttttcggcggagggaccaagctgaccgtccta

SEQ ID NO: 144 - DNA, 384 bp, Artificial Sequence
  source: /note=“Description of Artificial Sequence Synthetic polynucleotide”
gaggtgcagctggtgcagtctggggcagaggtgaaaaagcccggagagtctctgaagatctcctgtaagggctctggatacacctttaccaaccactggatcgcctgggtgcgccagatgcccgggaaaggcctggagtggatgggcatcatctatcctggtgactctgaaacgaggtacagcccgtccttccaaggccacgtcaccatctcagccgacaagtccatcagtaccgcctatttgcagtggagcaccctgaaggactcggactccgccatgtacttctgtgtgagacaggcccgtggctgggacgacggacgggctggatattattattccggtatggacgcctggggccagggaaccctggtcaccgtctcgagt

SEQ ID NO: 145 - DNA, 330 bp, Artificial Sequence
  source: /note=“Description of Artificial Sequence Synthetic polynucleotide”
caggctgtggtgctccaggagccatcgttctcagtgtcccctggagggacagtcacactcacctgtggcttgcgctctgggtcagtctctactagtcactaccccagctggtaccagcagaccccaggccaggctccacgcacgctcatttacagcacaaacactcgctcttctggggtccctgatcgcttctctggctccatccttgggaacaaagctgccctcaccatcacgggggcccaggcagatgatgaatctaattattactgtatgctatacatgggcagtggcatgtatgtgttcggcggagggaccaaggtcaccgtccta

SEQ ID NO: 146 - DNA, 360 bp, Artificial Sequence
  source: /note=“Description of Artificial Sequence Synthetic polynucleotide”
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgagagtcagcgggagccactttccattctttgactcctggggccaggggacaatggtcaccgtctcgagt

SEQ ID NO: 147 - DNA, 324 bp, Artificial Sequence
  source: /note=“Description of Artificial Sequence Synthetic polynucleotide”
cagtctgtgctgactcagccaccctcggtgtcagtggccccaggacagacggccagaattacctgtgggggagacaagattggacataaaagtgtgcattggtatcagcagaagccaggccaggcccctgtgttgctcgtctatgatgataggaagcggccctcagggatccctgagcgattctctggctccaactctgggaacacggccaccctgaccatcagcagggtcgaggccggggatgaggctgcctatcactgtcaggtgtgggatagaagtagtgacccttatgtcttcggaactgggaccaaggtcaccgtccta

SEQ ID NO: 148 - DNA, 357 bp, Artificial Sequence
  source: /note=“Description of Artificial Sequence Synthetic polynucleotide”
caggtgcagctggtgcaatctggggctgaagtgaagaagcctggggcctcagtgaaggtctcttgtcaggcttctggatacaccttcagcgggcactatatgcacttggtgcgacaggcccctggacaagggcttgagtggatggggtggatccaccctaccagtggtggcacaacctatgcacagaagtttcagggccgggtcgttatgaccagggacacgtccatcagcacagcctacatggaactgagtaggctgacatctgacgacacggccgtgtattactgtgcaagaatgtcccaaaactatgatgcttttgatatctggggccaagggacaatggtcaccgtctcgagt

SEQ ID NO: 149 - DNA, 327 bp, Artificial Sequence
  source: /note=“Description of Artificial Sequence Synthetic polynucleotide”
caggctgtgctgactcagccgtcctcagtgtctggggccccagggcagagggtcaccatctcctgcactgggagcagctccaacatcggggcaggttatgatgtaaactggtaccaacaatttccaggaacagcccccaaaattatcgtctatggcgatcggccctcaggggcccctgaccgattctctggctccaagtctggcacctcagcctccctggcaatcactggactccgggctgaggatgaggctgattattactgccagtcctgggacagtcgcctgagtagttatgtcttcggaactgggaccaaggtcaccgtccta

SEQ ID NO: 150 - DNA, 369 bp, Artificial Sequence
  source: /note=“Description of Artificial Sequence Synthetic polynucleotide”
caggtgcagctgcaggagtcggggggaggcgtggtccagcctggggggtccctgagactctcctgtgcagcgtctggattcaccttcagtggctatggcatgcactgggtccgccaggctccaggcaaggggctggagtgggtggcatctgtacggaacgatggaagtaatacatactacacagactccgtgaaggaccgattcaccatctccagagacaacaccaagaacacgctgtatctgcaaatgaacagcctgagagccgaggacacggccgtatattactgtgccaagtcgagaagagtgatgtatggcacctcctattactttgactactggggcagaggcaccctggtcaccgtctcctca

SEQ ID NO: 151 - DNA, 324 bp, Artificial Sequence; identical to SEQ ID NO: 125
  source: /note=“Description of Artificial Sequence Synthetic polynucleotide”

SEQ ID NO: 152 - DNA, 378 bp, Artificial Sequence
  source: /note=“Description of Artificial Sequence Synthetic polynucleotide”
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgagagatctgggaatagaccccctttggagtggttattacacaccccttgactattggggccgagggacaatggtcaccgtctcgagt

SEQ ID NO: 153 - DNA, 330 bp, Artificial Sequence
  source: /note=“Description of Artificial Sequence Synthetic polynucleotide”
cacgttatactgactcaaccgccctcagcgtctgggacccccgggcagagggtcaccatctcttgttctggaagcagctccaacatcggaagtaattccgttagctggtaccagcagctcccaggaacggcccccaaactcctcatgtatactaacaatcagcggccctcaggggtccctgaccgattctctggctccaagtctggcacctcagcctccctggccatcagtgggctccagtctgaggatgaggctgattattactgtgcgacatgggatgccagcctgaatacttgggtgttcggcggagggaccaaggtcaccgtccta

SEQ ID NO: 154 - DNA, 348 bp, Artificial Sequence
  source: /note=“Description of Artificial Sequence Synthetic polynucleotide”
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgagaggcgggagtgggagtgactactggggccaggggacaatggtcaccgtctcgagt

SEQ ID NO: 155 - DNA, 330 bp, Artificial Sequence
  source: /note=“Description of Artificial Sequence Synthetic polynucleotide”
aattttatgctgactcagccccactctgtgtcggggtctccggggaagacggtaaccatctcctgcacccgcagcagtggctacattgacagcaagtatgtgcagtggtaccagcagcgcccgggcagtgcccccaccactgtgatctatgaggataaccgaagaccctctggggtccctgatcggttctctggctccatcgacagctcctccaactctgcctccctcaccatctctggactggagactgaggacgaggctgactattactgtcagtcttatgatgacaccaatgtggtgttcggcggagggaccaaggtcaccgtccta

SEQ ID NO: 156 - DNA, 360 bp, Artificial Sequence
  source: /note=“Description of Artificial Sequence Synthetic polynucleotide”
gaggtccagctggtgcagtctggagctgaggtgaaggagcctggggcctcagtgaaggtctcctgcaaggcctctggttacgacttttccaactatggtttcagctgggtgcgccaggcccctggacaaggtcttgagtggatgggatggatcagctcttataatggttacacaaactatgcacagagactccagggcagagtcaccatgaccacagacacatccacgagcacagcctacatggagctgaggagcctgagatctgacgacacagctgtctattactgtgcgagagatcgaggacttggaaactggtacttcgatctctggggccaaggcaccctggtcaccgtctcgagt

SEQ ID NO: 157 - DNA, 330 bp, Artificial Sequence; identical to SEQ ID NO: 127
  source: /note=“Description of Artificial Sequence Synthetic polynucleotide”

SEQ ID NO: 158 - DNA, 1518 bp, Artificial Sequence
  source: /note=“Description of Artificial Sequence Synthetic polynucleotide”
atggattttcaagtgcagattttcagcttcctgctaatcagtgcttcagtcataatgtccagaggagatattcagatgacccagagcccgagcagcctgagcgcgagcgtgggcgatcgcgtgaccattacctgccgcgcgagccaggatgtgaacaccgcggtggcgtggtatcagcagaaaccgggcaaagcgccgaaactgctgatttatagcgcgagctttctgtatagcggcgtgccgagccgctttagcggcagccgcagcggcaccgattttaccctgaccattagcagcctgcagccggaagattttgcgacctattattgccagcagcattataccaccccgccgacctttggccagggcaccaaagtggaaattaaacgcaccgggggtggaggctctggtggcggtggctctggcggaggtggatccggtggcggcggatctgaagtgcagctggtggaaagcggcggcggcctggtgcagccgggcggcagcctgcgcctgagctgcgcggcgagcggctttaacattaaagatacctatattcattgggtgcgccaggcgccgggcaaaggcctggaatgggtggcgcgcatttatccgaccaacggctatacccgctatgcggatagcgtgaaaggccgctttaccattagcgcggataccagcaaaaacaccgcgtatctgcagatgaacagcctgcgcgcggaagataccgcggtgtattattgcagccgctggggcggcgatggcttttatgcgatggattattggggccagggcaccctggtgaccgtgagcagtgatcaggagcccaaatcttgtgacaaaactcacacatctccaccgtgctcagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 159 - protein, 505 aa, Artificial Sequence
  source: /note=“Description of Artificial Sequence Synthetic polypeptide”
MDFQVQIFSFLLISASVIMSRGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGGGGSGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSDQEPKSCDKTHTSPPCSAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 160 - DNA, 66 bp, Artificial Sequence
  source: /note=“Description of Artificial Sequence Synthetic oligonucleotide”
atggattttcaagtgcagattttcagcttcctgctaatcagtgcttcagtcataatgtccagagga

SEQ ID NO: 161 - protein, 22 aa, Artificial Sequence
  source: /note=“Description of Artificial Sequence Synthetic peptide”
MDFQVQIFSFLLISASVIMSRG

SEQ ID NO: 162 - DNA, 327 bp, Artificial Sequence
  source: /note=“Description of Artificial Sequence Synthetic polynucleotide”
gatattcagatgacccagagcccgagcagcctgagcgcgagcgtgggcgatcgcgtgaccattacctgccgcgcgagccaggatgtgaacaccgcggtggcgtggtatcagcagaaaccgggcaaagcgccgaaactgctgatttatagcgcgagctttctgtatagcggcgtgccgagccgctttagcggcagccgcagcggcaccgattttaccctgaccattagcagcctgcagccggaagattttgcgacctattattgccagcagcattataccaccccgccgacctttggccagggcaccaaagtggaaattaaacgcacc

SEQ ID NO: 163 - protein, 109 aa, Artificial Sequence
  source: /note=“Description of Artificial Sequence Synthetic polypeptide”
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRT

SEQ ID NO: 164 - DNA, 60 bp, Artificial Sequence
  source: /note=“Description of Artificial Sequence Synthetic oligonucleotide”
gggggtggaggctctggtggcggtggctctggcggaggtggatccggtggcggcggatct

SEQ ID NO: 165 - protein, 20 aa, Artificial Sequence
  source: /note=“Description of Artificial Sequence Synthetic peptide”
GGGGSGGGGSGGGGSGGGGS

SEQ ID NO: 166 - DNA, 360 bp, Artificial Sequence
  source: /note=“Description of Artificial Sequence Synthetic polynucleotide”
gaagtgcagctggtggaaagcggcggcggcctggtgcagccgggcggcagcctgcgcctgagctgcgcggcgagcggctttaacattaaagatacctatattcattgggtgcgccaggcgccgggcaaaggcctggaatgggtggcgcgcatttatccgaccaacggctatacccgctatgcggatagcgtgaaaggccgctttaccattagcgcggataccagcaaaaacaccgcgtatctgcagatgaacagcctgcgcgcggaagataccgcggtgtattattgcagccgctggggcggcgatggcttttatgcgatggattattggggccagggcaccctggtgaccgtgagcagt

SEQ ID NO: 167 - protein, 120 aa, Artificial Sequence [sequence omitted]

SEQ ID NO: 168 - DNA, 45 bp, Artificial Sequence
  source: /note=“Description of Artificial Sequence Synthetic oligonucleotide”
gagcccaaatcttgtgacaaaactcacacatctccaccgtgctca

SEQ ID NO: 169 - protein, 15 aa, Artificial Sequence
  source: /note=“Description of Artificial Sequence Synthetic peptide”
EPKSCDKTHTSPPCS

SEQ ID NO: 170 - DNA, 654 bp, Artificial Sequence
  source: /note=“Description of Artificial Sequence Synthetic polynucleotide”
gcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 171 - protein, 217 aa, Artificial Sequence
  source: /note=“Description of Artificial Sequence Synthetic polypeptide”
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 172 - DNA, 1503 bp, Artificial Sequence
  source: /note=“Description of Artificial Sequence Synthetic polynucleotide”
atggaagcaccagcgcagcttctcttcctcctgctactctggctcccagataccaccggtgaggtgcagctggtgcagtctgggtctgaggtgaggaggcctgggtcctcggtgagggtctcctgcacggcttctggagacacctccagcagctttaccgtcaactggctgcgacaggcccctggacaaggtcttgagtggatgggagggatcacccctatgtttggcactgcaaactacgcacagatgttcgaggacagagtcacgataaccgcggacgaaatggaactgagtggcctgacatctgaggacacggccgtgtatttttgtgcgacaggcccctccgattacgtttgggggagttatcgtttccttgacacctgggggcgggggaccacggtcaccgtctcgagtggaggcggcggttcaggcggaggtggctctggcggtggcggaagtgcacaggctgtgctgactcagccgtcctcagtgtctgcggccccaggacaggaggtctccatctcctgctctggagccagatccaacgttgggggtaattatgtttcctggtaccaacacctcccaggaacagcccccaaactcctcatttatgacaataataagcgaccctcagggatgcctgaccgattctctggctccaagtctggcacgtcagccaccctgggcatcaccggagtccagactgaggacgaggccgattattactgcgcaacatgggatagcagcctgagcgctgtggtcttcggcggagggaccaagctgaccgtcctaggtgacgtacgcgagcccaaatcttctgacaaaactcacacatgcccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 173 - protein, 500 aa, Artificial Sequence
  source: /note=“Description of Artificial Sequence Synthetic polypeptide”
MEAPAQLLFLLLLWLPDTTGEVQLVQSGSEVRRPGSSVRVSCTASGDTSSSFTVNWLRQAPGQGLEWMGGITPMFGTANYAQMFEDRVTITADEMELSGLTSEDTAVYFCATGPSDYVWGSYRFLDTWGRGTTVTVSSGGGGSGGGGSGGGGSAQAVLTQPSSVSAAPGQEVSISCSGARSNVGGNYVSWYQHLPGTAPKLLIYDNNKRPSGMPDRFSGSKSGTSATLGITGVQTEDEADYYCATWDSSLSAVVFGGGTKLTVLGDVREPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 174 - DNA, 1503 bp, Artificial Sequence
  source: /note=“Description of Artificial Sequence Synthetic polynucleotide”
atggaagcaccagcgcagcttctcttcctcctgctactctggctcccagataccaccggtcaggtgcagctggtgcagtctgggtctgaggtgaggaggcctgggtcctcggtgaggatctcctgcacggcttctggagacacctccagcagctttaccgtcaactgggtgcgacaggcccctggacaaggtcttgagtggatgggagggatcacccctatgtttggcactgcaaactacgcacaggtgttcgaggacagagtcacaataatcgcggacgagatggaactgagtggcctgacatctgaggacacggccgtgtatttctgtgcgacaggcccctccgattacgtttgggggagttatcgtttccttgacaactggggcaggggcaccctggtcaccgtctcgagtggaggcggcggttcaggcggaggtggctctggcggtggcggaagtgcacagtctgtgctgactcagccaccctcagtgtctgcggccccagggcagaaggtcaccatctcctgctctggaggcaggtccagcattgggaataattatgtgtcctggtatcaacacctcccaggaacagcccccaaactcctcatctatgacaataatcagcgaccctcagggattcctgaccgattctctggctccaagtctggcacgtcagccaccctgggcatcaccggactccagactggggacgaggccgattattactgcggaacatgggatagcagcctgagtgctgtggtgtttggcggagggaccaaggtcaccgtcctaggtgacgtacgcgagcccaaatcttctgacaaaactcacacatgcccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 175 - protein, 500 aa, Artificial Sequence
  source: /note=“Description of Artificial Sequence Synthetic polypeptide”
MEAPAQLLFLLLLWLPDTTGQVQLVQSGSEVRRPGSSVRISCTASGDTSSSFTVNWVRQAPGQGLEWMGGITPMFGTANYAQVFEDRVTIIADEMELSGLTSEDTAVYFCATGPSDYVWGSYRFLDNWGRGTLVTVSSGGGGSGGGGSGGGGSAQSVLTQPPSVSAAPGQKVTISCSGGRSSIGNNYVSWYQHLPGTAPKLLIYDNNQRPSGIPDRFSGSKSGTSATLGITGLQTGDEADYYCGTWDSSLSAVVFGGGTKVTVLGDVREPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 176 - DNA, 1515 bp, Artificial Sequence
  source: /note=“Description of Artificial Sequence Synthetic polynucleotide”
atggaagcaccagcgcagcttctcttcctcctgctactctggctcccagataccaccggtgaagtgcagctggtgcagtctggggctgaggtgaagaagcctggggcctcagtgaaggtctcctgcaaggcttctgggtacagcttcaccgccttctatattcactgggtgcgacaggcccctggacaaggccttgagtatttgggatggatcgaccctaatactggtgccacaaaatatgcacagcgctttcagggcagggtcatcatgacctgggacacgtccatcaccacagccaccatggaactgagcaggctgacgtctgacgactcggccgtctactactgtgtgagagatttgcgggagtggggctacgaattgtccgttgagtattggggcagaggaaccctggtcaccgtctcgagtggaggcggcggttcaggcggaggtggctctggcggtggcggaagtgcacagtctgtgctgactcagccaccctcagcgtctgggacccccgggcagagggtcaccatctcttgttctggaagcagctccaacatcggaagtaattatgtatactggtaccagcagctcccaggaacggcccccaaactcctcatctataggaataatcagcggccctcaggggtccctgaccgattctctggctccaagtctggcacctcagcctccctggccatcagtgggctccggtccgaggatgaggctgattattactgtgcagcatgggatgacagcctgagtggttgggtgttcggcggagggaccaagctgaccgtcctaggtgacgtacgcgagcccaaatcttctgacaaaactcacacatgcccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 177 - protein, 504 aa, Artificial Sequence
  source: /note=“Description of Artificial Sequence Synthetic polypeptide”
MEAPAQLLFLLLLWLPDTTGEVQLVQSGAEVKKPGASVKVSCKASGYSFTAFYIHWVRQAPGQGLEYLGWIDPNTGATKYAQRFQGRVIMTWDTSITTATMELSRLTSDDSAVYYCVRDLREWGYELSVEYWGRGTLVTVSSGGGGSGGGGSGGGGSAQSVLTQPPSASGTPGQRVTISCSGSSSNIGSNYVYWYQQLPGTAPKLLIYRNNQRPSGVPDRFSGSKSGTSASLAISGLRSEDEADYYCAAWDDSLSGWVFGGGTKLTVLGDVREPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 178 - DNA, 1503 bp, Artificial Sequence
  source: /note=“Description of Artificial Sequence Synthetic polynucleotide”
atggaagcaccagcgcagcttctcttcctcctgctactctggctcccagataccaccggtgaggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatgcacagaagtttcagggctgggtcaccatgaccagggacacgtccatcagcacagcctacatggagctgagcaggctgagatctgacgacacggccgtgtattactgtgcgagagattctactatggccccaggtgcttttgatatctggggccgaggcaccctggtcaccgtctcgagtggaggcggcggttcaggcggaggtggctctggcggtggcggaagtgcacagtctgtgctgactcagccaccctcggtgtcagtggccccaggacagacggccaggatgacctgtgggggaaacaacattgaaagtaaaactgtgcattggtaccagcagaagccgggccaggcccctgtgctggtcgtctacaatgataacgtccggccctcagggatccctgcgcgattctctggctccaactccggcaacacggccaccctgaccatcaacagggtcgaagccggggatgaggccgactattattgtcaggtgtgggactccagtagagatcaaggggtattcggcggagggaccaagctgaccgtcctaggtgacgtacgcgagcccaaatcttctgacaaaactcacacatgcccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 179 - protein, 500 aa, Artificial Sequence
  source: /note=“Description of Artificial Sequence Synthetic polypeptide”
MEAPAQLLFLLLLWLPDTTGEVQLVQSGAEVKKPGASVKVSCKASGYTFTGYYMHWVRQAPGQGLEWMGWINPNSGGTNYAQKFQGWVTMTRDTSISTAYMELSRLRSDDTAVYYCARDSTMAPGAFDIWGRGTLVTVSSGGGGSGGGGSGGGGSAQSVLTQPPSVSVAPGQTARMTCGGNNIESKTVHWYQQKPGQAPVLVVYNDNVRPSGIPARFSGSNSGNTATLTINRVEAGDEADYYCQVWDSSRDQGVFGGGTKLTVLGDVREPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 180 - DNA, 1503 bp, Artificial Sequence
  source: /note=“Description of Artificial Sequence Synthetic polynucleotide”
atggaagcaccagcgcagcttctcttcctcctgctactctggctcccagataccaccggtgaggtgcagctggtgcagtctgggggaggcttggtcaggcctggagggtccctgagactctcctgtgcagcctcgggattctccttcagtgactactacatgacctggatccgccagattccagggaaggggctggagtgggtggcagttatatggaatgatggaagtgatagatactatgcagactccgtgaagggccgattcaccatttccagagacaattccaagaacacgctgtttctgcaaatgagcagcctgagagacgaggacacggctctatattactgtgtgagagggggaccaacagcttcaagcggatttgactactggggccgaggcaccctggtcaccgtctcgagtggtggaggcggttcaggcggaggtggcagcggcggtggcggatcgtctgagctgactcagcctgcctccgtgtctgggtctcctggacagtcgatcaccatctcctgcactggaaccagcagtgacgttggtggttataactatgtctcctggtacctacaacacccaggcaaagcccccaaactcatgatttatgagggcagtaagcggccctcaggggtttctaatcgcttctctggctccaagtctggcaacacggcctccctgacaatctctgggctccaggctgaggacgaggctgattattactgcagctcatatacaaccaggagcactcgagttttcggcggagggaccaagctgaccgtcctaggtgacgtacgcgagcccaaatcttctgacaaaactcacacatgcccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 181 - protein, 500 aa, Artificial Sequence
  source: /note=“Description of Artificial Sequence Synthetic polypeptide”
MEAPAQLLFLLLLWLPDTTGEVQLVQSGGGLVRPGGSLRLSCAASGFSFSDYYMTWIRQIPGKGLEWVAVIWNDGSDRYYADSVKGRFTISRDNSKNTLFLQMSSLRDEDTALYYCVRGGPTASSGFDYWGRGTLVTVSSGGGGSGGGGSGGGGSSELTQPASVSGSPGQSITISCTGTSSDVGGYNYVSWYLQHPGKAPKLMIYEGSKRPSGVSNRFSGSKSGNTASLTISGLQAEDEADYYCSSYTTRSTRVFGGGTKLTVLGDVREPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 182 - DNA, 1503 bp, Artificial Sequence
  source: /note=“Description of Artificial Sequence Synthetic polynucleotide”
atggaagcaccagcgcagcttctcttcctcctgctactctggctcccagataccaccggtcaggtgcagctgcaggagtcgggtccaggactggtgaagccctcgcagaccttgtcactcacctgtggcatctccggggacagtgtctctagcaacagtgctgcttggaactggatcaggcagtccccaacgagaggccttgagtggctgggaaggacatattacaggtccagttggtatcataactatgcaccttctatgaacagtcgattaaccatcatcgcagacacatccaaaaaccagttctctttgcaactgaactctgtgactcccgaggacacggctgtatattactgtgcaagcgggtgggcctttgatgtctggggcaggggaaccctggtcaccgtctcgagtggaggcggcggttcaggcggaggtggctctggcggtggcggaagtgcacagtctgtgctgactcagccaccctccgcgtccgggtctcctggacagtcagtcaccatctcctgcactggaaccagcagtgacgttggtgcttatgactttgtctcctggtaccaacagcaccctggcaaagcccccaaactcatgatttatgaggtcaataagcggccctcaggggtccctgatcgcttctctggctccaagtctggcaacacggcctccctgaccgtctctgggctccaggctgaggatgaggctgattattactgcagctcatatgcaggcagcaagaatttgcttttcggcggagggaccaagctgaccgtcctaggtgacgtacgcgagcccaaatcttctgacaaaactcacacatgcccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 183 - protein, 500 aa, Artificial Sequence
  source: /note=“Description of Artificial Sequence Synthetic polypeptide”
MEAPAQLLFLLLLWLPDTTGQVQLQESGPGLVKPSQTLSLTCGISGDSVSSNSAAWNWIRQSPTRGLEWLGRTYYRSSWYHNYAPSMNSRLTIIADTSKNQFSLQLNSVTPEDTAVYYCASGWAFDVWGRGTLVTVSSGGGGSGGGGSGGGGSAQSVLTQPPSASGSPGQSVTISCTGTSSDVGAYDFVSWYQQHPGKAPKLMIYEVNKRPSGVPDRFSGSKSGNTASLTVSGLQAEDEADYYCSSYAGSKNLLFGGGTKLTVLGDVREPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 184 - DNA, 1506 bp, Artificial Sequence
  source: /note=“Description of Artificial Sequence Synthetic polynucleotide”
atggaagcaccagcgcagcttctcttcctcctgctactctggctcccagataccaccggtgaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgagaggatacagtggctacgatgaccctgactcctgggggagagggaccacggtcaccgtctcgagtggaggcggcggttcaggcggaggtggctctggcggtggcggaagtgcacacgttatactgactcaaccgccctcaacgtctgggacccccgggcagacggtcaccatctcttgttctgggagcagctccaacatcggaagtcattatgtatactggtaccagcagctcccaggaacggcccccaaactcctcatctataggaataatcagcggccctcaggggtccctgaccgattctctggctccaagtctggcacctcagcctccctggccatcagtgggctccggtccgaggatgagactgattattactgtgcagcatgggatgacagcctgagtggtcgagtcttcggaactgggaccaagctgaccgtcctaggtgacgtacgcgagcccaaatcttctgacaaaactcacacatgcccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 185 - protein, 501 aa, Artificial Sequence
  source: /note=“Description of Artificial Sequence Synthetic polypeptide”
MEAPAQLLFLLLLWLPDTTGEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGYSGYDDPDSWGRGTTVTVSSGGGGSGGGGSGGGGSAHVILTQPPSTSGTPGQTVTISCSGSSSNIGSHYVYWYQQLPGTAPKLLIYRNNQRPSGVPDRFSGSKSGTSASLAISGLRSEDETDYYCAAWDDSLSGRVFGTGTKLTVLGDVREPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 186 - DNA, 1497 bp, Artificial Sequence
  source: /note=“Description of Artificial Sequence Synthetic polynucleotide”
atggaagcaccagcgcagcttctcttcctcctgctactctggctcccagataccaccggtcaggtacagctgcagcagtcaggggctgaggtgaagaagcctgggtcctcggtgaaggtctcctgcaaggcttctggaggcaccatcagcaactatgctatcagttgggtgcggctggcccctggacaaggtcttgagtggatgggaagtatcgtccctcttcatgggacaacaaacttcgcacagaaattccagggcagagtcacgatcaccgcggacgagtccacgagcacatcctacatggaggtgaacgtcctgacatatgaagacacggcgatgtattattgtgcgtctctcaattggggctactggggccggggcaccctggtcaccgtctcgagtggaggcggcggttcaggcggaggtggctctggcggtggcggaagtgcacttaattttatgctgactcagccccactctgtgtcggagtctccggggaagacggtaaccatctcctgcaccggcagtagtggcagcattgccagcaactatgtgcagtggtaccagcagcgcccggacagtgcccccaccactgtgatctatgaggataatcgaagatcctctggagtccctgatcggttctctggctccatcgacagctcctccaactctgcctccctcagcatctctggactgaagactgaggacgaggctgactactactgtcagtcctatgatagtagcggtcatgtggtcttcggcggagggaccaagctgaccgtcctaggtgacgtacgcgagcccaaatcttctgacaaaactcacacatgcccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 187 - protein, 498 aa, Artificial Sequence
  source: /note=“Description of Artificial Sequence Synthetic polypeptide”
MEAPAQLLFLLLLWLPDTTGQVQLQQSGAEVKKPGSSVKVSCKASGGTISNYAISWVRLAPGQGLEWMGSIVPLHGTTNFAQKFQGRVTITADESTSTSYMEVNVLTYEDTAMYYCASLNWGYWGRGTLVTVSSGGGGSGGGGSGGGGSALNFMLTQPHSVSESPGKTVTISCTGSSGSIASNYVQWYQQRPDSAPTTVIYEDNRRSSGVPDRFSGSIDSSSNSASLSISGLKTEDEADYYCQSYDSSGHVVFGGGTKLTVLGDVREPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 188 - DNA, 1506 bp, Artificial Sequence
  source: /note=“Description of Artificial Sequence Synthetic polynucleotide”
atggaagcaccagcgcagcttctcttcctcctgctactctggctcccagataccaccggtgaggtgcagctggtgcagtctggggcagaggtgaaaaagcccggggagtctctgaagatctcctgtaagggttttggatacaattttcgcagcgcctggatcggctgggtgcgccagatgcccggcaaaggcctggagtggatgggggtcatctatcctggtgactctgatgtcagatacagtccgtccttccaaggccaggtcaccatctcagccgacaagtccatcagtaccgcctacctgcagtggagcagcctgaaagcctcggacaccgccatgtattattgtacgagacccgtagggcagtgggtggactctgactattggggcaagggaaccctggtcaccgtctcgagtggaggcggcggttcaggcggaggtggctctggcggtggcggaagtgcacagtctgtgttgacgcagccgccctcagcgtctgggacccccggacagagggtcaccatctcttgttctggaagcagctccaacatcggaactaatactgtgaactggtaccagcagcttccaggaacggcccccaaactcctcatctatactagtaatcagcggccctcaggggtccctgcccgcttctctgcctccaactctggcacctcagcctccctggccatcagtgggctccggtccgaggatgaggctgattattattgtgcagcgtgggatgacaagttgagtggtgcggtgttcggcggagggaccaagctgaccgtcctaggtgacgtacgcgagcccaaatcttctgacaaaactcacacatgcccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 189 - protein, 501 aa, Artificial Sequence
  source: /note=“Description of Artificial Sequence Synthetic polypeptide”
MEAPAQLLFLLLLWLPDTTGEVQLVQSGAEVKKPGESLKISCKGFGYNFRSAWIGWVRQMPGKGLEWMGVIYPGDSDVRYSPSFQGQVTISADKSISTAYLQWSSLKASDTAMYYCTRPVGQWVDSDYWGKGTLVTVSSGGGGSGGGGSGGGGSAQSVLTQPPSASGTPGQRVTISCSGSSSNIGTNTVNWYQQLPGTAPKLLIYTSNQRPSGVPARFSASNSGTSASLAISGLRSEDEADYYCAAWDDKLSGAVFGGGTKLTVLGDVREPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 190 - DNA, 1509 bp, Artificial Sequence
  source: /note=“Description of Artificial Sequence Synthetic polynucleotide”
atggaagcaccagcgcagcttctcttcctcctgctactctggctcccagataccaccggtgaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgagacagtcgggcgcggactggtacttcgatctctggggccgaggcaccctggtcaccgtctcgagtggaggcggcggttcaggcggaggtggctctggcggtggcggaagtgcacaggctgtgctgactcagccgtccgcagtttctggggccccagggcagagggtcaccatctcctgcactgggaccagctccaacatcgggacaaactatcttgtacactggtatcagcaacgtccaggaacagccccccaactcctcgtctctggtaacaacactcgaccctctggggtcactgaccggttctctgtctccaagtctgccacttcagcctccctggccatcactgggctccaggctgaggatgaggctgattattactgccagacctatgacatcaacttgagggtttgggtgttcggcggagggaccaaggtcaccgtcctaggtgacgtacgcgagcccaaatcttctgacaaaactcacacatgcccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 191 - protein, 502 aa, Artificial Sequence
  source: /note=“Description of Artificial Sequence Synthetic polypeptide”
MEAPAQLLFLLLLWLPDTTGEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARQSGADWYFDLWGRGTLVTVSSGGGGSGGGGSGGGGSAQAVLTQPSAVSGAPGQRVTISCTGTSSNIGTNYLVHWYQQRPGTAPQLLVSGNNTRPSGVTDRFSVSKSATSASLAITGLQAEDEADYYCQTYDINLRVWVFGGGTKVTVLGDVREPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 192 - DNA, 1491 bp, Artificial Sequence
  source: /note=“Description of Artificial Sequence Synthetic polynucleotide”
atggaagcaccagcgcagcttctcttcctcctgctactctggctcccagataccaccggtgaggtgcagctggtggagactgggggaggcgtggtccagcctggggggtccctgagcctctcctgtgcagcgtctggattcaccttcagtagctatggcatgcagtgggtccgccaggctccaggcaaggggctggagtgggtggcgtttatacggtacgatggaagtagtgaatactatgcagactccgtgaagggccgattcaccatctccagagacaattccaagaacacgctgtatctgcaaatgaacagcctgagagctgaggacacggctgtgtattactgtggaagaacgctggagtctagtttgtggggcaagggaaccctggtcaccgtctcgagtggtggaggcggttcaggcggaggtggcagcggcggtggcggatcgcagtctgtgttgacgcagccgccctcagtgtctgcggccccaggacagaaggtcaccatttcctgctctggaagcacctccaacattgggaataattatgtctcctggtaccaacagcacccaggcaaagcccccaaactcatgatttatgatgtcagtaagcggccctcaggggtccctgaccgattctctggctccaagtctggcaactcagcctccctggacatcagtgggctccagtctgaggatgaggctgattattactgtgcagcatgggatgacagcctgagtgaatttctcttcggaactaggaccaagctgaccgtcctaggtgacgtacgcgagcccaaatcttctgacaaaactcacacatgcccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 193 - protein, 496 aa, Artificial Sequence
  source: /note=“Description of Artificial Sequence Synthetic polypeptide”
MEAPAQLLFLLLLWLPDTTGEVQLVETGGGVVQPGGSLSLSCAASGFTFSSYGMQWVRQAPGKGLEWVAFIRYDGSSEYYADSVKGRFTISRDNSKNTLYLQMNSLRAEDTAVYYCGRTLESSLWGKGTLVTVSSGGGGSGGGGSGGGGSQSVLTQPPSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTRTKLTVLGDVREPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 194 - DNA, 1509 bp, Artificial Sequence
  source: /note=“Description of Artificial Sequence Synthetic polynucleotide”
atggaagcaccagcgcagcttctcttcctcctgctactctggctcccagataccaccggtcaggtgcagctggtgcagtctggagctgaggtgaagaagcctgggtcctcggtgaaggtctcctgcaaggcttctggttacacctttaccagctatggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggccgtgtattactgtgcgagagtcccgggcgtaagtgggagctatccagactactactacatggacgtctggggcaagggaaccctggtcaccgtctcctcaggtggaggcggttcaggcggtggcagcggcggtggcggatcggacatccagatgacccagtctccttccaccctgtctgcatctattggagacagagtcaccatcacctgccgggccagtgagggtatttatcactggttggcctggtatcagcagaagccagggaaagctcctaaactcctgatctataaggcctctagtttagccagtggggccccatcaaggttcagcggcagtggatctgggacagatttcactctcaccatcagcagcctgcagcctgatgattttgcaacttattactgccaacaatatagtaattatccgctcactttcggcggagggaccaagctggagatcaaacgtgacgtacgcgagcccaaatcttctgacaaaactcacacatgcccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 195 - protein, 502 aa, Artificial Sequence
  source: /note=“Description of Artificial Sequence Synthetic polypeptide”
MEAPAQLLFLLLLWLPDTTGQVQLVQSGAEVKKPGSSVKVSCKASGYTFTSYGISWVRQAPGQGLEWMGWISAYNGNTNYAQKLQGRVTMTTDTSTSTAYMELRSLRSDDTAVYYCARVPGVSGSYPDYYYMDVWGKGTLVTVSSGGGGSGGGSGGGGSDIQMTQSPSTLSASIGDRVTITCRASEGIYHWLAWYQQKPGKAPKLLIYKASSLASGAPSRFSGSGSGTDFTLTISSLQPDDFATYYCQQYSNYPLTFGGGTKLEIKRDVREPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 196 - DNA, 1509 bp, Artificial Sequence
  source: /note=“Description of Artificial Sequence Synthetic polynucleotide”
atggaagcaccagcgcagcttctcttcctcctgctactctggctcccagataccaccggtgaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgagatggaggcctcttctagactaccactttgaccaatggggccaagggacaatggtcaccgtctcgagtggaggcggcggttcaggcggaggtggctctggcggtggcggaagtgcacagtctgtgctgactcagccaccctcagcgtctgggacccccggacagacggtaacaatctcttgttctggaagcagctccaacatcggaagtagtgttgttaattggtaccagcagttcccaggaacggcccccaaagtcctcgtctatagtaacactcagcggccctcaggggtccctgaccgattctctggctccaggtctggcacctcagcctccctggccatcagtgggctccagtctgaggatgaggctgattattactgtttagcatgggatgccagcctgaatggttgggtgttcggcggagggaccaagctgaccgtcctaggtgacgtacgcgagcccaaatcttctgacaaaactcacacatgcccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 197 - protein, 502 aa, Artificial Sequence
  source: /note=“Description of Artificial Sequence Synthetic polypeptide”
MEAPAQLLFLLLLWLPDTTGEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARWRPLLDYHFDQWGQGTMVTVSSGGGGSGGGGSGGGGSAQSVLTQPPSASGTPGQTVTISCSGSSSNIGSSVVNWYQQFPGTAPKVLVYSNTQRPSGVPDRFSGSRSGTSASLAISGLQSEDEADYYCLAWDASLNGWVFGGGTKLTVLGDVREPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 198 - DNA, 1509 bp, Artificial Sequence
  source: /note=“Description of Artificial Sequence Synthetic polynucleotide”
atggaagcaccagcgcagcttctcttcctcctgctactctggctcccagataccaccggtgaggtgcagctggtggagactgggggaggcttggtacagcctggggggtccctgagactctcctgtgcagcctctggattcaccttcagtagctatggcatgaactgggtccgccaggctccagggaaggggctggagtgggtttcatacattagtagttctggtaataccatattctacgcagactctgtgaagggccgattcaccatctccagagacagtgccaagaattcagtgtctctgcagatgaacagcctgagagacgaggacacggctgtgtattactgtgcttcctactactcctactactacggtatggacgcctggggccaggggacaatggtcaccgtctcgagtggaggcggcggttcaggcggaggtggctctggcggtggcggaagtgcactttcctatgtgctgactcagccaccctcagcgtctgggacccccgggcagagggtcaccatctcttgttctggaagcagctccaacatcggaagtaatactgtaaactggtaccagcagctcccaggaacggcccccaaactcctcatctatagtaataatcagcggccctcaggggtccctgaccgattctctggctccaagtctggcacctcagcctccctggccatcagtgggctgcggtccgaggatgaggctgattattactgtgcagcatgggattacagcctgagtggttgggtgttcggcggagggaccaaggtcaccgtcctaggtgacgtacgcgagcccaaatcttctgacaaaactcacacatgcccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 199 - protein, 502 aa, Artificial Sequence
  source: /note=“Description of Artificial Sequence Synthetic polypeptide”
MEAPAQLLFLLLLWLPDTTGEVQLVETGGGLVQPGGSLRLSCAASGFTFSSYGMNWVRQAPGKGLEWVSYISSSGNTIFYADSVKGRFTISRDSAKNSVSLQMNSLRDEDTAVYYCASYYSYYYGMDAWGQGTMVTVSSGGGGSGGGGSGGGGSALSYVLTQPPSASGTPGQRVTISCSGSSSNIGSNTVNWYQQLPGTAPKLLIYSNNQRPSGVPDRFSGSKSGTSASLAISGLRSEDEADYYCAAWDYSLSGWVFGGGTKVTVLGDVREPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 200 - DNA, 1497 bp, Artificial Sequence
  source: /note=“Description of Artificial Sequence Synthetic polynucleotide”
atggaagcaccagcgcagcttctcttcctcctgctactctggctcccagataccaccggtgaggtgcagctggtggagactggggaaggcctggtcaagcctggggggtccctgagactctcctgtacagcctctggattcaccttcaggagttatagcttgaactgggtccgccaggctccagggcaggggctggagtgggtctcatccattagtagtactagtacttacatatactacgcagactcggtgaagggccgattcaccatctccagagacgacgccaagaacacactgtatctgcaaatgaacagcctgagagccgaagacacagctgcatattactgtgttagactgggatctggtgggggatattttcctgactactggggcaggggcaccctggtcaccgtctcgagtggtggaggcggttcaggcggaggtggcagcggcggtggcggatcgtctgagctgactcaggaccctgctgtgtctgtggccttgggacagacagtcaggatcacatgccaaggagacagcctcagaagctattatgcaagctggtaccagcagaagccaggacaggcccctgtacttgtcatctatggtaaaaacaaccggccctcagggatcccagaccgattctctggctccagctcaggaaacacagcttccttgaccatcactggggctcaggcggaagatgaggctgactattactgtaactcccgggacagcagtggtaaccatgtggtattcggcggagggaccaagctgaccgtcctaggtgacgtacgcgagcccaaatcttctgacaaaactcacacatgcccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 201 - protein, 498 aa, Artificial Sequence [sequence omitted]

SEQ ID NO: 202 - DNA, 1509 bp, Artificial Sequence
  source: /note=“Description of Artificial Sequence Synthetic polynucleotide”
atggaagcaccagcgcagcttctcttcctcctgctactctggctcccagataccaccggtcaggtgcagctggtggagtctggggctgaggtgaagaagcctggggcctcagtgaaggtctcctgcaaggcttctggatacaccttcaccagttatgatatcaactgggtgcgacaggcccccggacaaaggcttgagtggatgggatggatcaacgctggcaatggtaacacaaaatattcacagaagttccagggcagagtcaccattaccagggacacatccgcgagcacagcctacatggagctgaggagcctgagatctgacgacacggccgtgtattactgtgcgagagggaggagctatggccacccgtactactttgactactggggccagggaaccctggtcaccgtctcgagtggtggaggcggttcaggcggaggtggcagcggcggtggcggatcgcagtctgtgctgactcagcctgcctccgtgtctgggtctcctggacagtcgatcaccatctcctgcactggaaccagcagtgacgttggtggttataactatgtctcctggtaccaacaacacccaggcaaagcccccaaactcatgatttatgagggcagtaagcggccctcaggggtttctaatcgcttctctggctccaagtctggcaacacggcctccctgacaatctctgggctccaggctgaggacgaggctgattattactgcagctcatatacaaccaggagcactcgagttttcggcggagggaccaagctgaccgtcctaggtgacgtacgcgagcccaaatcttctgacaaaactcacacatgcccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 203 - protein, 502 aa, Artificial Sequence
  source: /note=“Description of Artificial Sequence Synthetic polypeptide”
MEAPAQLLFLLLLWLPDTTGQVQLVESGAEVKKPGASVKVSCKASGYTFTSYDINWVRQAPGQRLEWMGWINAGNGNTKYSQKFQGRVTITRDTSASTAYMELRSLRSDDTAVYYCARGRSYGHPYYFDYWGQGTLVTVSSGGGGSGGGGSGGGGSQSVLTQPASVSGSPGQSITISCTGTSSDVGGYNYVSWYQQHPGKAPKLMIYEGSKRPSGVSNRFSGSKSGNTASLTISGLQAEDEADYYCSSYTTRSTRVFGGGTKLTVLGDVREPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 204 - DNA, 1518 bp, Artificial Sequence
  source: /note=“Description of Artificial Sequence Synthetic polynucleotide”
atggaagcaccagcgcagcttctcttcctcctgctactctggctcccagataccaccggtaaggtgcagctggtgcagtctgggacagaggtgaaaaagcccggggagtctctgaagatctcctgtcagggttctggatacaggtttagtagtgactggattgcctgggtgcgccagatgcccgggaaaggcctggagtggatggggattgtctatcctggtgactctgataccagatatagcccgtccttccaaggccaagtcaccatctcagccgacaagtccatcagtactgcctacctgcagtggagcggcctgaaggcctcggacaccgccaagtattactgtgcgagagtgcaacaggcagtgggagctaaaggttatgctatggacgtctggggcaagggaaccctggtcaccgtctcgagtggaggcggcggttcaggcggaggtggctctggcggtggcggaagtgcacagactgtggtgatccaggagccatcgttctcagtgtcccctggagggacagtcacactcacttgtggcttgagctctggctcagtctctaccagttactaccccagctggtaccggcagaccccaggccaggctccacacacactcattcacaacacaaagattcgctcctctggggtccctgatcgcttctctggctccatccttgggaacaatgctgccctcaccatcacgggggcccaggcagatgatgaatctgattattactgtcttttgtatatgggtagcggcatttacgtgttcggcggagggaccaagctgaccgtcctaggtgacgtacgcgagcccaaatcttctgacaaaactcacacatgcccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 205 - protein, 505 aa, Artificial Sequence
  source: /note=“Description of Artificial Sequence Synthetic polypeptide”
MEAPAQLLFLLLLWLPDTTGKVQLVQSGTEVKKPGESLKISCQGSGYRFSSDWIAWVRQMPGKGLEWMGIVYPGDSDTRYSPSFQGQVTISADKSISTAYLQWSGLKASDTAKYYCARVQQAVGAKGYAMDVWGKGTLVTVSSGGGGSGGGGSGGGGSAQTVVIQEPSFSVSPGGTVTLTCGLSSGSVSTSYYPSWYRQTPGQAPHTLIHNTKIRSSGVPDRFSGSILGNNAALTITGAQADDESDYYCLLYMGSGIYVFGGGTKLTVLGDVREPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 206 - DNA, 1518 bp, Artificial Sequence
  source: /note=“Description of Artificial Sequence Synthetic polynucleotide”
atggaagcaccagcgcagcttctcttcctcctgctactctggctcccagataccaccggtgaagtgcagctggtgcagtctggggctgaggtgaagaagcctggggcctcagtgagggtctcctgcaagggttctggaaacaccttcaccggccactacatccactgggtgcgacaggcccctggacaaggacttgagtggctgggatggatcgaccctaacactggtgacatacagtattcagaaaactttaagggctcggtcaccttgaccagggacccatccatcaactcagtcttcatggacctgatcaggctgacatctgacgacacggccatgtattactgtgcgagagaaggtgccgggctcgccaactactattactacggtctggacgtctggggccgagggacaatggtcaccgtctcgagtggaggcggcggttcaggcggaggtggctctggcggtggcggaagtgcacagactgtggtgctccaggagccttcgttctcagtgtcccctggggggacagtcacactcacttgtggcttgaactttggctcagtctctactgcttactaccccagttggtaccagcagaccccaggccaagctccacgcacgctcatctacggcacaaatattcgttcctctggggtcccggatcgcttctctggctccatcgtagggaacaaagctgccctcaccatcacgggggcccagacagaagatgagtctgattattattgtgcgctgtatatgggtagtggcatgctcttcggcggcgggaccaaggtcaccgtcctaggtgacgtacgcgagcccaaatcttctgacaaaactcacacatgcccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 207 - protein, 505 aa, Artificial Sequence
  source: /note=“Description of Artificial Sequence Synthetic polypeptide”
MEAPAQLLFLLLLWLPDTTGEVQLVQSGAEVKKPGASVRVSCKGSGNTFTGHYIHWVRQAPGQGLEWLGWIDPNTGDIQYSENFKGSVTLTRDPSINSVFMDLIRLTSDDTAMYYCAREGAGLANYYYYGLDVWGRGTMVTVSSGGGGSGGGGSGGGGSAQTVVLQEPSFSVSPGGTVTLTCGLNFGSVSTAYYPSWYQQTPGQAPRTLIYGTNIRSSGVPDRFSGSIVGNKAALTITGAQTEDESDYYCALYMGSGMLFGGGTKVTVLGDVREPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 208 - DNA, 1503 bp, Artificial Sequence
  source: /note=“Description of Artificial Sequence Synthetic polynucleotide”
atggaagcaccagcgcagcttctcttcctcctgctactctggctcccagataccaccggtgaagtgcagctggtgcagtctggggctgaagtgaagaagcctggggcctcagtgaaggtctcttgtcaggcttctggatacaccttcagcgggcactatatgcacttggtgcgacaggcccctggacaagggcttgagtggatggggtggatccaccctaccagtggtggcacaacctatgcacagaagtttcagggccgggtcgttatgaccagggacacgtccatcagcacagcctacatggaactgagtaggctgacatctgacgacacggccgtgtattactgtgcaagaatgtcccaaaactatgatgcttttgatatctggggccaagggacaatggtcaccgtctcgagtggaggcggcggttcaggcggaggtggctctggcggtggcggaagtgcacaggctgtgctgactcagccgtcctcagtgtctggggccccagggcagagggtcaccatctcctgcactgggagcagctccaacatcggggcaggttatgatgtaaactggtaccaacaatttccaggaacagcccccaaaattatcgtctatggcgatcggccctcaggggcccctgaccgattctctggctccaagtctggcacctcagcctccctggcaatcactggactccgggctgaggatgaggctgattattactgccagtcctgggacagtcgcctgagtagttatgtcttcggaactgggaccaaggtcaccgtcctaggtgacgtacgcgagcccaaatcttctgacaaaactcacacatgcccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 209 - protein, 500 aa, Artificial Sequence
  source: /note=“Description of Artificial Sequence Synthetic polypeptide”
MEAPAQLLFLLLLWLPDTTGEVQLVQSGAEVKKPGASVKVSCQASGYTFSGHYMHLVRQAPGQGLEWMGWIHPTSGGTTYAQKFQGRVVMTRDTSISTAYMELSRLTSDDTAVYYCARMSQNYDAFDIWGQGTMVTVSSGGGGSGGGGSGGGGSAQAVLTQPSSVSGAPGQRVTISCTGSSSNIGAGYDVNWYQQFPGTAPKIIVYGDRPSGAPDRFSGSKSGTSASLAITGLRAEDEADYYCQSWDSRLSSYVFGTGTKVTVLGDVREPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 210 - DNA, 1533 bp, Artificial Sequence
  source: /note=“Description of Artificial Sequence Synthetic polynucleotide”
atggaagcaccagcgcagcttctcttcctcctgctactctggctcccagataccaccggtgaggtgcagctggtgcagtctggggcagaggtgaaaaagcccggagagtctctgaagatctcctgtaagggctctggatacacctttaccaaccactggatcgcctgggtgcgccagatgcccgggaaaggcctggagtggatgggcatcatctatcctggtgactctgaaacgaggtacagcccgtccttccaaggccacgtcaccatctcagccgacaagtccatcagtaccgcctatttgcagtggagcaccctgaaggactcggactccgccatgtacttctgtgtgagacaggcccgtggctgggacgacggacgggctggatattattattccggtatggacgcctggggccagggaaccctggtcaccgtctcgagtggaggcggcggttcaggcggaggtggctctggcggtggcggaagtgcacaggctgtggtgctccaggagccatcgttctcagtgtcccctggagggacagtcacactcacctgtggcttgcgctctgggtcagtctctactagtcactaccccagctggtaccagcagaccccaggccaggctccacgcacgctcatttacagcacaaacactcgctcttctggggtccctgatcgcttctctggctccatccttgggaacaaagctgccctcaccatcacgggggcccaggcagatgatgaatctaattattactgtatgctatacatgggcagtggcatgtatgtgttcggcggagggaccaaggtcaccgtcctaggtgacgtacgcgagcccaaatcttctgacaaaactcacacatgcccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 211 - protein, 510 aa, Artificial Sequence
  source: /note=“Description of Artificial Sequence Synthetic polypeptide”
MEAPAQLLFLLLLWLPDTTGEVQLVQSGAEVKKPGESLKISCKGSGYTFTNHWIAWVRQMPGKGLEWMGIIYPGDSETRYSPSFQGHVTISADKSISTAYLQWSTLKDSDSAMYFCVRQARGWDDGRAGYYYSGMDAWGQGTLVTVSSGGGGSGGGGSGGGGSAQAVVLQEPSFSVSPGGTVTLTCGLRSGSVSTSHYPSWYQQTPGQAPRTLIYSTNTRSSGVPDRFSGSILGNKAALTITGAQADDESNYYCMLYMGSGMYVFGGGTKVTVLGDVREPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 212 - DNA, 1527 bp, Artificial Sequence
  source: /note=“Description of Artificial Sequence Synthetic polynucleotide”
atggaagcaccagcgcagcttctcttcctcctgctactctggctcccagataccaccggtgaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgagagatctgggaatagaccccctttggagtggttattacacaccccttgactattggggccgagggacaatggtcaccgtctcgagtggaggcggcggttcaggcggaggtggctctggcggtggcggaagtgcacacgttatactgactcaaccgccctcagcgtctgggacccccgggcagagggtcaccatctcttgttctggaagcagctccaacatcggaagtaattccgttagctggtaccagcagctcccaggaacggcccccaaactcctcatgtatactaacaatcagcggccctcaggggtccctgaccgattctctggctccaagtctggcacctcagcctccctggccatcagtgggctccagtctgaggatgaggctgattattactgtgcgacatgggatgccagcctgaatacttgggtgttcggcggagggaccaaggtcaccgtcctaggtgacgtacgcgagcccaaatcttctgacaaaactcacacatgcccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 213 - protein, 508 aa, Artificial Sequence
  source: /note=“Description of Artificial Sequence Synthetic polypeptide”
MEAPAQLLFLLLLWLPDTTGEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARDLGIDPLWSGYYTPLDYWGRGTMVTVSSGGGGSGGGGSGGGGSAHVILTQPPSASGTPGQRVTISCSGSSSNIGSNSVSWYQQLPGTAPKLLMYTNNQRPSGVPDRFSGSKSGTSASLAISGLQSEDEADYYCATWDASLNTWVFGGGTKVTVLGDVREPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 214 - DNA, 1506 bp, Artificial Sequence
  source: /note=“Description of Artificial Sequence Synthetic polynucleotide”
atggaagcaccagcgcagcttctcttcctcctgctactctggctcccagataccaccggtgaggtccagctggtgcagtctggagctgaggtgaaggagcctggggcctcagtgaaggtctcctgcaaggcctctggttacgacttttccaactatggtttcagctgggtgcgccaggcccctggacaaggtcttgagtggatgggatggatcagctcttataatggttacacaaactatgcacagagactccagggcagagtcaccatgaccacagacacatccacgagcacagcctacatggagctgaggagcctgagatctgacgacacagctgtctattactgtgcgagagatcgaggacttggaaactggtacttcgatctctggggccaaggcaccctggtcaccgtctcgagtggtggaggcggttcaggcggaggtggcagcggcggtggcggatcgcagtctgtgctgactcagcctgcctccgtgtctgggtctcctggacagtcgatcaccatctcctgcactggaaccagcagtgacgttggtggttataactatgtctcctggtaccaacaacacccaggcaaagcccccaaactcatgatttatgagggcagtaagcggccctcaggggtttctaatcgcttctctggctccaagtctggcaacacggcctccctgacaatctctgggctccaggctgaggacgaggctgattattactgcagctcatatacaaccaggagcactcgagttttcggcggagggaccaagctgaccgtcctaggtgacgtacgcgagcccaaatcttctgacaaaactcacacatgcccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 215 - protein, 501 aa, Artificial Sequence
  source: /note=“Description of Artificial Sequence Synthetic polypeptide”
MEAPAQLLFLLLLWLPDTTGEVQLVQSGAEVKEPGASVKVSCKASGYDFSNYGFSWVRQAPGQGLEWMGWISSYNGYTNYAQRLQGRVTMTTDTSTSTAYMELRSLRSDDTAVYYCARDRGLGNWYFDLWGQGTLVTVSSGGGGSGGGGSGGGGSQSVLTQPASVSGSPGQSITISCTGTSSDVGGYNYVSWYQQHPGKAPKLMIYEGSKRPSGVSNRFSGSKSGNTASLTISGLQAEDEADYYCSSYTTRSTRVFGGGTKLTVLGDVREPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 216 - DNA, 1509 bp, Artificial Sequence
  source: /note=“Description of Artificial Sequence Synthetic polynucleotide”
atggaagcaccagcgcagcttctcttcctcctgctactctggctcccagataccaccggtcagatgcagctggtgcagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtattaaatactatgcagactccgtgaagggccgattcaccatctccagagacaattccaagaacacactgtatctacaaatgaacagcctgagagccgaggacacgggcgtttattactgttcgaaagatcgctatagcagtggctggtacagctccgatgcttttgatatttggggccgagggacaatggtcaccgtctcgagtggtggaggcggttcaggcggaggtggcagcggcggtggcggatcgtctgagctgactcaggaccctgctgtgtctgtggccttgggacagacagtcaggatcacatgccaaggagacagcctcagaagctattatgcaagctggtaccagcagaagccaggacaggcccctgtacttgtcatctatggtaaaaacaaccggccctcagggatcccagaccgattctctggctccagctcaggaaacacagcttccttgaccatcactggggctcaggcggaagatgaggctgactattactgtcattcccgggacagcagtggtaaccatgtgcttttcggcggagggaccaagctgaccgtcctaggtgacgtacgcgagcccaaatcttctgacaaaactcacacatgcccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 217 - protein, 502 aa, Artificial Sequence
  source: /note=“Description of Artificial Sequence Synthetic polypeptide”
MEAPAQLLFLLLLWLPDTTGQMQLVQSGGGVVQPGRSLRLSCAASGFTFSSYGMHWVRQAPGKGLEWVAVISYDGSIKYYADSVKGRFTISRDNSKNTLYLQMNSLRAEDTGVYYCSKDRYSSGWYSSDAFDIWGRGTMVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCHSRDSSGNHVLFGGGTKLTVLGDVREPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 218 - DNA, 1515 bp, Artificial Sequence
  source: /note=“Description of Artificial Sequence Synthetic polynucleotide”
atggaagcaccagcgcagcttctcttcctcctgctactctggctcccagataccaccggtgaggtgcagctggtgcagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtattaaatactatgcagactccgtgaagggccgattcaccatctccagagacaattccaagaacacgctgtatctgcaaatgaacagcctgagagctgaggacacggctgtgtattactgtgcgcgaactggtgaatatagtggctacgatacgagtggttacagcaattggggccaaggcaccctggtcaccgtctcgagtggtggaggcggttcaggcggaggtggcagcggcggtggcggatcgcagtctgtgctgactcagccaccctcagcgtctgggacccccgggcagagggtcaccatctcttgttctggaagcagctccaacatcgggagtaacactgtaaactggtaccagcgactcccaggagcggccccccaactcctcatctacaataatgaccagcggccctcagggatccctgaccgattctctggctccaagtctggcacctcaggctccctggtcatcagtgggctccagtctgaagatgaggctgattactactgtgcgtcatgggatgacagtctgaatggtcgggtgttcggcggagggaccaagctgaccgtcctaggtgacgtacgcgagcccaaatcttctgacaaaactcacacatgcccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 219 - protein, 504 aa, Artificial Sequence
  source: /note=“Description of Artificial Sequence Synthetic polypeptide”
MEAPAQLLFLLLLWLPDTTGEVQLVQSGGGVVQPGRSLRLSCAASGFTFSSYGMHWVRQAPGKGLEWVAVISYDGSIKYYADSVKGRFTISRDNSKNTLYLQMNSLRAEDTAVYYCARTGEYSGYDTSGYSNWGQGTLVTVSSGGGGSGGGGSGGGGSQSVLTQPPSASGTPGQRVTISCSGSSSNIGSNTVNWYQRLPGAAPQLLIYNNDQRPSGIPDRFSGSKSGTSGSLVISGLQSEDEADYYCASWDDSLNGRVFGGGTKLTVLGDVREPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 220 - DNA, 1488 bp, Artificial Sequence
  source: /note=“Description of Artificial Sequence Synthetic polynucleotide”
atggaagcaccagcgcagcttctcttcctcctgctactctggctcccagataccaccggtcaggtgcagctggtgcagtctgggggaggcttggtccagccgggggggtccctgagactctcctgtgcagcctctggattcacgtttagtacctatgccatgagttgggcccgccaggctccagggaaggggctggagtgggtctcaagtattagtggtgatggtggaagaattctcgatgcagactccgcgaagggccggttcaccatctccagagacaattccaagaacacgctgtatctgcaaatgaacggcctgagagtcgaggacacggccctttattactgtgcgagagcggacggtaactactggggcagggggacaatggtcaccgtctcttcaggtggaggcggttcaggcggaggtggcagcggcggtggcggatcgcagtctgtgctgactcagcctgcctccgtgtctgggtctcctggacagtcgatcaccatctcctgcactggaaccagcagtgacgttggtggttataactatgtctcctggtaccaacaacacccaggcaaagcccccaaactcatgatttatgagggcagtaagcggccctcaggggtttctaatcgcttctctggctccaagtctggcaacacggcctccctgacaatctctgggctccaggctgaggacgaggctgattattactgcagctcatatacaaccaggagcactcgagttttcggcggagggaccaagctgaccgtcctaggtgacgtacgcgagcccaaatcttctgacaaaactcacacatgcccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 221 - protein, 495 aa, Artificial Sequence
  source: /note=“Description of Artificial Sequence Synthetic polypeptide”
MEAPAQLLFLLLLWLPDTTGQVQLVQSGGGLVQPGGSLRLSCAASGFTFSTYAMSWARQAPGKGLEWVSSISGDGGRILDADSAKGRFTISRDNSKNTLYLQMNGLRVEDTALYYCARADGNYWGRGTMVTVSSGGGGSGGGGSGGGGSQSVLTQPASVSGSPGQSITISCTGTSSDVGGYNYVSWYQQHPGKAPKLMIYEGSKRPSGVSNRFSGSKSGNTASLTISGLQAEDEADYYCSSYTTRSTRVFGGGTKLTVLGDVREPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 222 - DNA, 1506 bp, Artificial Sequence
  source: /note=“Description of Artificial Sequence Synthetic polynucleotide”
atggaagcaccagcgcagcttctcttcctcctgctactctggctcccagataccaccggtcaggtgcagctgcaggagtcggggggaggcgtggtccagcctggggggtccctgagactctcctgtgcagcgtctggattcaccttcagtggctatggcatgcactgggtccgccaggctccaggcaaggggctggagtgggtggcatctgtacggaacgatggaagtaatacatactacacagactccgtgaaggaccgattcaccatctccagagacaacaccaagaacacgctgtatctgcaaatgaacagcctgagagccgaggacacggccgtatattactgtgccaagtcgagaagagtgatgtatggcacctcctattactttgactactggggcagaggcaccctggtcaccgtctcctcaggtggaggcggttcaggcggaggtggcagcggcggtggcggatcgtctgagctgactcaggaccctgctgtgtctgtggccttgggacagacagtcaggatcacatgccaaggagacagcctcagaagctattatgcaagctggtaccagcagaagccaggacaggcccctgtacttgtcatctatggtaaaaacaaccggccctcagggatcccagaccgattctctggctccagctcaggaaacacagcttccttgaccatcactggggctcaggcggaagatgaggctgactattactgtaactcccgggacagcagtggtaaccatgtggtattcggcggagggaccaagctgaccgtcctaggtgacgtacgcgagcccaaatcttctgacaaaactcacacatgcccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 223 - protein, 501 aa, Artificial Sequence
  source: /note=“Description of Artificial Sequence Synthetic polypeptide”
MEAPAQLLFLLLLWLPDTTGQVQLQESGGGVVQPGGSLRLSCAASGFTFSGYGMHWVRQAPGKGLEWVASVRNDGSNTYYTDSVKDRFTISRDNTKNTLYLQMNSLRAEDTAVYYCAKSRRVMYGTSYYFDYWGRGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGDVREPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 224 - DNA, 1503 bp, Artificial Sequence
  source: /note=“Description of Artificial Sequence Synthetic polynucleotide”
atggaagcaccagcgcagcttctcttcctcctgctactctggctcccagataccaccggtcaggtgcagctgcaggagtcgggcgcaggactggtgaagccttcggggaccctgtccctcacctgcgctgtctctggtggctccatcagcagtggtaactggtggagttgggtccgccagcccccagggaaggggctggagtggattggggaaatctctcatagtgggagcaccaactacaacccgtccctcaagagtcgagtcaccatatcagtagacaagtccaagaaccagttctccctgaacctgagttctgtgaccgccgcagacacggccgtgtattactgtgcgagagtaaggggtacggtgggggatacacggggacctgactactggggccagggaaccctggtcaccgtctcgagtggtggaggcggttcaggcggaggtggcagcggcggtggcggatcgtctgagctgactcaggaccctgctgtgtctgtggccttgggacagacagtcaggatcacatgccaaggagacagcctcagaagctattatgcaagctggtaccagcagaagccaggacaggcccctgtacttgtcatctatggtaaaaacaaccggccctcagggatcccagaccgattctctggctccagctcaggaaacacagcttccttgaccatcactggggctcaggcggaagatgaggctgactattactgtaactcccgggacagcagtggtaaccatgtggtattcggcggagggaccaagctgaccgtcctaggtgacgtacgcgagcccaaatcttctgacaaaactcacacatgcccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 225 - protein, 500 aa, Artificial Sequence
  source: /note=“Description of Artificial Sequence Synthetic polypeptide”
MEAPAQLLFLLLLWLPDTTGQVQLQESGAGLVKPSGTLSLTCAVSGGSISSGNWWSWVRQPPGKGLEWIGEISHSGSTNYNPSLKSRVTISVDKSKNQFSLNLSSVTAADTAVYYCARVRGTVGDTRGPDYWGQGTLVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLGDVREPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 226 - DNA, 1503 bp, Artificial Sequence
  source: /note=“Description of Artificial Sequence Synthetic polynucleotide”
atggaagcaccagcgcagcttctcttcctcctgctactctggctcccagataccaccggtgaggtgcagctggtgcagtctgggggaggcctggtcaagcctggggggtccctgagactctcctgtgcagcgtctggattcaccttcagtagctatgggatgcactgggtccgccaggctccaggcaaggggctggagtgggtggcaggtattttttatgatggaggtaataaatactatgcagactccgtgaagggccgattcaccatctccagagacaattccaagaacacgctgtatctgcaaatgaacagcctgagagctgaggacacggctgtgtattactgtgcgagagataggggctactactacatggacgtctggggcaaagggaccacggtcaccgtctcctcaggtggaggcggttcaggcggaggtggctctggcggtggcggatcgcagtctgtgttgacgcagccgccctcagtgtctggggccccaggacagagggtcaccatctcctgcactgggagaagctccaacatcggggcgggtcatgatgtacactggtaccagcaacttccaggaacagcccccaaactcctcatctatggtgacagcaatcggccctcaggggtccctgaccgattctctggctccaggtctggcacctcagcctccctggccatcactgggctccaggctgaagatgaggctgattattactgccagtcctatgacagcagcctgaggggttcggtattcggcggagggaccaaggtcaccgtcctaggtgacgtacgcgagcccaaatcttctgacaaaactcacacatgcccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 227 - protein, 500 aa, Artificial Sequence
  source: /note=“Description of Artificial Sequence Synthetic polypeptide”
MEAPAQLLFLLLLWLPDTTGEVQLVQSGGGLVKPGGSLRLSCAASGFTFSSYGMHWVRQAPGKGLEWVAGIFYDGGNKYYADSVKGRFTISRDNSKNTLYLQMNSLRAEDTAVYYCARDRGYYYMDVWGKGTTVTVSSGGGGSGGGGSGGGGSQSVLTQPPSVSGAPGQRVTISCTGRSSNIGAGHDVHWYQQLPGTAPKLLIYGDSNRPSGVPDRFSGSRSGTSASLAITGLQAEDEADYYCQSYDSSLRGSVFGGGTKVTVLGDVREPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 228 - DNA, 1500 bp, Artificial Sequence
  source: /note=“Description of Artificial Sequence Synthetic polynucleotide”
atggaagcaccagcgcagcttctcttcctcctgctactctggctcccagataccaccggtgaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgagaggcgggagtgggagtgactactggggccaggggacaatggtcaccgtctcgagtggaggcggcggttcaggcggaggtggctctggcggtggcggaagtgcacttaattttatgctgactcagccccactctgtgtcggggtctccggggaagacggtaaccatctcctgcacccgcagcagtggctacattgacagcaagtatgtgcagtggtaccagcagcgcccgggcagtgcccccaccactgtgatctatgaggataaccgaagaccctctggggtccctgatcggttctctggctccatcgacagctcctccaactctgcctccctcaccatctctggactggagactgaggacgaggctgactattactgtcagtcttatgatgacaccaatgtggtgttcggcggagggaccaaggtcaccgtcctaggtgacgtacgcgagcccaaatcttctgacaaaactcacacatgcccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 229 - protein, 499 aa, Artificial Sequence
  source: /note=“Description of Artificial Sequence Synthetic polypeptide”
MEAPAQLLFLLLLWLPDTTGEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGGSGSDYWGQGTMVTVSSGGGGSGGGGSGGGGSALNFMLTQPHSVSGSPGKTVTISCTRSSGYIDSKYVQWYQQRPGSAPTTVIYEDNRRPSGVPDRFSGSIDSSSNSASLTISGLETEDEADYYCQSYDDTNVVFGGGTKVTVLGDVREPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 230 - DNA, 1500 bp, Artificial Sequence
  source: /note=“Description of Artificial Sequence Synthetic polynucleotide”
atggaagcaccagcgcagcttctcttcctcctgctactctggctcccagataccaccggtggggtgcagctggtggagtctgggggaggcctggtcaagcctggggggtccctgagactctcctgtgcagcctctggattcaccttcagtagctataacatgaactgggtccgccaggctccagggaagggactggagtgggtctcagctattagtggtagtggtggtagcacatactacgcagactccgtgacgggccggttcaccatctccagagacaattccaagaacacgctgtatctgcaaatgaacagcctgagagccgaggacacggccgtatattactgtgcgaaagataccagtggctggtacggggacggtatggacgtctggggccggggaaccctggtcaccgtctcgagtggtggaggcggttcaggcggaggtggcagcggcggtggcggatcggacatccagatgacccagtctccttccaccctgtctgcatctattggagacagagtcaccatcacctgccgggccagtgagggtatttatcactggttggcctggtatcagcagaagccagggaaagcccctaaactcctgatctataaggcctctagtttagccagtggggccccatcaaggttcagcggcagtggatcagggacagatttcactctcaccatcagcagcctgcagcctgatgattttgcaacttattactgccaacaatatagtaattatccgctcactttcggcggagggaccaagctggagatcaaacgtgacgtacgcgagcccaaatcttctgacaaaactcacacatgcccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 231 - protein, 499 aa, Artificial Sequence
  source: /note=“Description of Artificial Sequence Synthetic polypeptide”
MEAPAQLLFLLLLWLPDTTGGVQLVESGGGLVKPGGSLRLSCAASGFTFSSYNMNWVRQAPGKGLEWVSAISGSGGSTYYADSVTGRFTISRDNSKNTLYLQMNSLRAEDTAVYYCAKDTSGWYGDGMDVWGRGTLVTVSSGGGGSGGGGSGGGGSDIQMTQSPSTLSASIGDRVTITCRASEGIYHWLAWYQQKPGKAPKLLIYKASSLASGAPSRFSGSGSGTDFTLTISSLQPDDFATYYCQQYSNYPLTFGGGTKLEIKRDVREPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 232 - DNA, 1503 bp, Artificial Sequence
  source: /note=“Description of Artificial Sequence Synthetic polynucleotide”
atggaagcaccagcgcagcttctcttcctcctgctactctggctcccagataccaccggtgaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcgagagtcagcgggagccactttccattctttgactcctggggccaggggacaatggtcaccgtctcgagtggaggcggcggttcaggcggaggtggctctggcggtggcggaagtgcacagtctgtgctgactcagccaccctcggtgtcagtggccccaggacagacggccagaattacctgtgggggagacaagattggacataaaagtgtgcattggtatcagcagaagccaggccaggcccctgtgttgctcgtctatgatgataggaagcggccctcagggatccctgagcgattctctggctccaactctgggaacacggccaccctgaccatcagcagggtcgaggccggggatgaggctgcctatcactgtcaggtgtgggatagaagtagtgacccttatgtcttcggaactgggaccaaggtcaccgtcctaggtgacgtacgcgagcccaaatcttctgacaaaactcacacatgcccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 233 - protein, 500 aa, Artificial Sequence [sequence omitted]

SEQ ID NO: 234 - DNA, 60 bp, Artificial Sequence
  source: /note=“Description of Artificial Sequence Synthetic oligonucleotide”
atggaagcaccagcgcagcttctcttcctcctgctactctggctcccagataccaccggt

SEQ ID NO: 235 - protein, 20 aa, Artificial Sequence
  source: /note=“Description of Artificial Sequence Synthetic peptide”
MEAPAQLLFLLLLWLPDTTG

SEQ ID NO: 236 - DNA, 45 bp, Artificial Sequence
  source: /note=“Description of Artificial Sequence Synthetic oligonucleotide”
ggaggcggcggttcaggcggaggtggctctggcggtggcggaagt

SEQ ID NO: 237 - protein, 15 aa, Artificial Sequence
  source: /note=“Description of Artificial Sequence Synthetic peptide”
GGGGSGGGGSGGGGS

SEQ ID NO: 238 - DNA, 45 bp, Artificial Sequence
  source: /note=“Description of Artificial Sequence Synthetic oligonucleotide”
gagcccaaatcttctgacaaaactcacacatgcccaccgtgccca

SEQ ID NO: 239 - protein, 15 aa, Artificial Sequence
  source: /note=“Description of Artificial Sequence Synthetic peptide”
EPKSSDKTHTCPPCP

SEQ ID NO: 240 - DNA, 708 bp, Artificial Sequence
  source: /note=“Description of Artificial Sequence Synthetic polynucleotide”
gacgtacgcgagcccaaatcttctgacaaaactcacacatgcccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 241 - protein, 235 aa, Artificial Sequence
  source: /note=“Description of Artificial Sequence Synthetic polypeptide”
DVREPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 242 - protein, 656 aa, Artificial Sequence
  source: /note=“Description of Artificial Sequence Synthetic polypeptide”
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTSIIS

SEQ ID NO: 243 - protein, 647 aa, Artificial Sequence
  source: /note=“Description of Artificial Sequence Synthetic polypeptide”
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQR

SEQ ID NO: 244 - protein, 599 aa, Artificial Sequence
  source: /note=“Description of Artificial Sequence Synthetic polypeptide”
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVAR

SEQ ID NO: 245 - protein, 338 aa, Artificial Sequence
  source: /note=“Description of Artificial Sequence Synthetic polypeptide”
MELAALCRWGLLLALLPPGAASTQVCTGTDMKLRLPASPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPC

SEQ ID NO: 246 - protein, 1255 aa, Homo sapiens [sequence omitted]

SEQ ID NO: 247 - DNA, 20 bp, Artificial Sequence
  source: /note=“Description of Artificial Sequence Synthetic primer”
ggaaacagctatgaccatga

SEQ ID NO: 248 - DNA, 21 bp, Artificial Sequence
  source: /note=“Description of Artificial Sequence Synthetic primer”
ctcttctgagatgagtttttg

SEQ ID NO: 249 - DNA, 18 bp, Artificial Sequence
  source: /note=“Description of Artificial Sequence Synthetic primer”
ggagattttcaacgtgaa

SEQ ID NO: 250 - DNA, 21 bp, Artificial Sequence
  source: /note=“Description of Artificial Sequence Synthetic primer”
ctcttctgagatgagtttttg

SEQ ID NO: 251 - protein, 5 aa, Artificial Sequence
  source: /note=“Description of Artificial Sequence Synthetic peptide”
GGGGS

SEQ ID NO: 252 - protein, 97 aa, Artificial Sequence
  source: /note=“Description of Artificial Sequence Synthetic polypeptide”
QVQLVQSGAEVKKPGASVKVSCKASGYTFTSYGISWVRQAPGQGLEWMGWISAYNGNTNYAQKQGRVTMTTDTSTSTAYMELRSLRSDDTAVYYCAR

SEQ ID NO: 253 - protein, 98 aa, Artificial Sequence
  source: /note=“Description of Artificial Sequence Synthetic polypeptide”
QVQLVQSGAEVKKPGASVKVSCKASGYTFTSYAMHWVRQAPGQRLEWMGWINAGNGNTKYSQKFQGRVTITRDTSASTAYMELSSLRSEDTAVYYCAR

SEQ ID NO: 254 - protein, 98 aa, Artificial Sequence
  source: /note=“Description of Artificial Sequence Synthetic polypeptide”
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAK

SEQ ID NO: 255 - protein, 118 aa, Artificial Sequence; identical to SEQ ID NO: 3
  source: /note=“Description of Artificial Sequence Synthetic polypeptide”

SEQ ID NO: 256 - protein, 101 aa, Artificial Sequence
  source: /note=“Description of Artificial Sequence Synthetic polypeptide”
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAR

SEQ ID NO: 257 - protein, 98 aa, Artificial Sequence
  source: /note=“Description of Artificial Sequence Synthetic polypeptide”
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCAR

SEQ ID NO: 258 - protein, 98 aa, Artificial Sequence
  source: /note=“Description of Artificial Sequence Synthetic polypeptide”
QVQLQESGPGLVKPSGTLSLTCAVSGGSISSSNWWSWVRQPPGKGLEWIGEIYHSGSTNYNPSLKSRVTISVDKSKNQFSLKLSSVTAADTAVYYCAR

SEQ ID NO: 259 - protein, 98 aa, Artificial Sequence
  source: /note=“Description of Artificial Sequence Synthetic polypeptide”
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCAR

SEQ ID NO: 260 - protein, 98 aa, Artificial Sequence
  source: /note=“Description of Artificial Sequence Synthetic polypeptide”
EVQLVESGGGLVQPGGSLRLSCAASGFTFSSYSMNWVRQAPGKGLEWVSYISSSSSTIYYADSVKGRFTISRDNAKNSLYLQMNSLRDEDTAVYYCAR

SEQ ID NO: 261 - protein, 98 aa, Artificial Sequence
  source: /note=“Description of Artificial Sequence Synthetic polypeptide”
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCAR

SEQ ID NO: 262 - protein, 98 aa, Artificial Sequence
  source: /note=“Description of Artificial Sequence Synthetic polypeptide”
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAK

SEQ ID NO: 263 - protein, 98 aa, Artificial Sequence
  source: /note=“Description of Artificial Sequence Synthetic polypeptide”
QVQLVQSGAEVKKPGASVKVSCKASGYTFTGYYMHWVRQAPGQGLEWMGWINPNSGGTNYAQKFQGWVTMTRDTSISTAYMELSRLRSDDTAVYYCAR

SEQ ID NO: 264 - protein, 92 aa, Artificial Sequence
  source: /note=“Description of Artificial Sequence Synthetic polypeptide”
DIQMTQSPSTLSASVGDRVTITCRASQSISSWLAWYQQKPGKAPKLLIYKASSLESGVPSRFSGSGSGTEFTLTISSLQPDDFATYYCQQYN

SEQ ID NO: 265 - protein, 93 aa, Artificial Sequence
  source: /note=“Description of Artificial Sequence Synthetic polypeptide”
QSVLTQPPSASGTPGQRVTISCSGSSSNIGSNTVNWYQQLPGTAPKLLIYSNNQRPSGVPDRFSGSKSGTSASLAISGLQSEDEADYYCAAWD

SEQ ID NO: 266 - protein, 112 aa, Artificial Sequence
  source: /note=“Description of Artificial Sequence Synthetic polypeptide”
QSVLTQPPSASGTPGQRVTISCSGSSSNIGSNTVNWYQRLPGAAPQLLIYNNDQRPSGIPDRFSGSKSGTSGSLVISGLQSEDEADYYCASWDDSLNGRVFGGGTKLTVLGA

SEQ ID NO: 267 - protein, 94 aa, Artificial Sequence
  source: /note=“Description of Artificial Sequence Synthetic polypeptide”
QTVVTQEPSFSVSPGGTVTLTCGLSSGSVSTSYYPSWYQQTPGQAPRTLIYSTNTRSSGVPDRFSGSILGNKAALTITGAQADDESDYYCVLYM

SEQ ID NO: 268 - protein, 93 aa, Artificial Sequence
  source: /note=“Description of Artificial Sequence Synthetic polypeptide”
NFMLTQPHSVSESPGKTVTISCTRSSGSIASNYVQWYQQRPGSSPTTVIYEDNQRPSGVPDRFSGSIDSNSASLTISGLKTEDEADYYCQSYD

SEQ ID NO: 269 - protein, 91 aa, Artificial Sequence
  source: /note=“Description of Artificial Sequence Synthetic polypeptide”
SSELTQDPAVSVALGQTVRITCQGDSLRSYYASWYQQKPGQAPVLVIYGKNNRPSGIPDRFSGSSSGNTASLTITGAQAEDEADYYCNSRD

SEQ ID NO: 270 - protein, 91 aa, Artificial Sequence
  source: /note=“Description of Artificial Sequence Synthetic polypeptide”
SYVLTQPPSVSVAPGQTARITCGGNNIGSKSVHWYQQKPGQAPVLVVYDDSDRPSGIPERFSGSNSGNTATLTISRVEAGDEADYYCQVWD

SEQ ID NO: 271 - protein, 94 aa, Artificial Sequence
  source: /note=“Description of Artificial Sequence Synthetic polypeptide”
QSALTQPASVSGSPGQSITISCTGTSSDVGGYNYVSWYQQHPGKAPKLMIYEVSNRPSGVSNRFSGSKSGNTASLTISGLQAEDEADYYCSSYT

SEQ ID NO: 272 - protein, 94 aa, Artificial Sequence
  source: /note=“Description of Artificial Sequence Synthetic polypeptide”
QSALTQPPSASGSPGQSVTISCTGTSSDVGGYNYVSWYQQHPGKAPKLMIYEVSKRPSGVPDRFSGSKSGNTASLTVSGLQAEDEADYYCSSYA

SEQ ID NO: 273 - protein, 94 aa, Artificial Sequence
  source: /note=“Description of Artificial Sequence Synthetic polypeptide”
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYD

SEQ ID NO: 274 - protein, 93 aa, Artificial Sequence
  source: /note=“Description of Artificial Sequence Synthetic polypeptide”
QSVLTQPPSVSAAPGQKVTISCSGSSSNIGNNYVSWYQQLPGTAPKLLIYDNNKRPSGIPDRFSGSKSGTSATLGITGLQTGDEADYYCGTWD

SEQ ID NO: 275 - protein, 93 aa, Artificial Sequence
  source: /note=“Description of Artificial Sequence Synthetic polypeptide”
QSVLTQPPSASGTPGQRVTISCSGSSSNIGSNYVYWYQQLPGTAPKLLIYRNNQRPSGVPDRFSGSKSGTSASLAISGLRSEDEADYYCAAWD

SEQ ID NO: 276 - protein, 6 aa, Artificial Sequence
  source: /note=“Description of Artificial Sequence Synthetic 6xHis tag”
HHHHHH

(19) United States
(12) Patent Application Publication
(10) Pub. No.: US 2012/0121505 A1
(43) Pub. Date: May 17, 2012

(54) THERAPEUTIC COMPOSITIONS AND METHODS

(76) Inventors: Davinder Singh Gill, Andover, MA (US); Laird Bloom, Needham, MA (US); Maximillian T. Follettie, Belmont, MA (US); Fionnuala McAleese, Cambridge, MA (US); Sreekumar Kodangattil, Westbrook, CT (US); John Francis DiJoseph, Woodbridge, NJ (US); Nitin K. Damle, Upper Saddle River, NJ (US); Peter R. Baum, Seattle, WA (US); Peter A. Thompson, Bellevue, WA (US); John C. Kumer, Seattle, WA (US); Alan F. Wahl, Mercer Island, WA (US); Paul A. Algate, Issaquah, WA (US); Sateesh Kumar Natarajan, Redmond, WA (US)

(21) Appl. No.: 13/349,481

(22) Filed: Jan. 12, 2012

Related U.S. Application Data
(63) Continuation of application No. 12/156,159, filed on May 29, 2008, now abandoned.
(60) Provisional application No. 60/932,302, filed on May 29, 2007.

(51) Int. Cl. A61K 39/395; C12N 15/13; A61K 49/00; A61K 51/10; C12P 21/08; C12N 1/19; C12N 1/21; C12N 5/10; C07K 16/30; A61P 35/00

(52) U.S. Cl. 424/1.49; 530/387.3; 536/23.53; 424/135.1; 424/9.6; 435/69.6; 435/7.1; 435/7.92; 435/328; 435/254.2; 435/252.33; 435/419; 424/9.1

(57) ABSTRACT

The present application provides novel binding proteins, including human binding proteins that specifically bind to the human ErbB2.

### CROSS REFERENCE TO RELATED APPLICATIONS

This application claims the benefit of U.S. Provisional Application Ser. No. 60/932,302, filed May 29, 2007.

### FIELD OF THE INVENTION

This invention relates to binding proteins that bind erythroblastic leukemia viral oncogene homolog 2 (ErbB2), in particular, human ErbB2 (also known as HER2), and their use in regulating ErbB2-associated activities. The binding proteins disclosed herein are useful in diagnosing, preventing, and/or treating ErbB2 associated disorders, e.g., hyperproliferative disorders, including cancer, and autoimmune disorders, including arthritis.

### BACKGROUND OF THE INVENTION

The ErbB family of receptor tyrosine kinases are important mediators of cell growth, differentiation and survival. The receptor family includes four distinct members including epidermal growth factor receptor (EGFR or ErbB1), HER2 (ErbB2 or p185^{neu}), HER3 (ErbB3) and HER4 (ErbB4 or tyro2). Structurally, the ErbB receptors possess an extracellular domain (with four subdomains, I-IV), a single hydrophobic transmembrane domain, and (except for HER3) a highly conserved tyrosine kinase domain. Crystal structures of EGFR reveal a receptor that adopts one of two conformations. In the “closed” conformation, EGFR is not bound by ligand and the extracellular subdomains II and IV remain tightly apposed, preventing inter-receptor interactions. Ligand binding prompts the receptor to adopt an “open” conformation, in which the EGFR receptor is poised to make inter-receptor interactions.

The ErbB receptors are generally found in various combinations in cells and heterodimerization is thought to increase the diversity of cellular responses to a variety of ErbB ligands. EGFR is bound by at least six different ligands; epidermal growth factor (EGF), transforming growth factor alpha (TGF-α), amphiregulin, heparin binding epidermal growth factor (HB-EGF), betacellulin and epiregulin. A family of heregulin proteins resulting from alternative splicing of a single gene are ligands for ErbB3 and ErbB4. The heregulin family includes alpha, beta and gamma heregulins, neu differentiation factors (NDFs), glial growth factors (GGFs); acetylcholine receptor inducing activity (ARIA); and sensory and motor neuron derived factor (SMDF).

HER2 was originally identified as the product of the transforming gene from neuroblastomas of chemically treated rats. The activated form of the neu proto-oncogene results from a point mutation (valine to glutamic acid) in the transmembrane region of the encoded protein. Amplification of the human homolog of neu is observed in breast and ovarian cancers and correlates with a poor prognosis. Overexpression of ErbB2 (frequently but not uniformly due to gene amplification) has also been observed in other carcinomas including carcinomas of the stomach, endometrium, salivary gland, lung, kidney, colon, thyroid, pancreas and bladder.

HER2 has been suggested to be a ligand orphan receptor. Ligand-dependent heterodimerization between HER2 and another HER family member, HER1, HER3 or HER4, activates the HER2 signaling pathway. The intracellular signaling pathway of HER2 is thought to involve ras-MAPK and PI3K pathways, as well as MAPK-independent S6 kinase and phospholipase C-gamma signaling pathways. HER2 signaling also effects proangiogenic factors, vascular endothelial growth factor (VEGF) and interleukin-8 (IL-8), and an antiangiogenic factor, thrombospondin-1 (TSP-1).

The full-length ErbB2 receptor undergoes proteolytic cleavage releasing its extracellular domain (ECD), which can be detected in cell culture medium and in patient's sera. The truncated ErbB2 receptor (p95ErbB2) that remains after proteolytic cleavage exhibits increased autokinase activity and transforming efficiency compared with the full-length receptor, implicating the ErbB2 ECD as a negative regulator of ErbB2 kinase and oncogenic activity.

A recombinant humanized version of the murine anti-ErbB2 antibody 4D5 (huMAb4D5-8, rhuMAb HER2 or HERCEPTIN®; U.S. Pat. No. 5,821,337) is clinically active in patients with ErbB2-overexpressing metastatic breast cancers that have received extensive prior anti-cancer therapy (Baselga et al., J. Clin. Oncol. 14:737-744 (1996)). HERCEPTIN® reportedly targets the C-terminal region of domain IV of ErbB2. HERCEPTIN® clinical activity is predominately dependent on antibody dependent cell mediated cytotoxicity (ADCC). Studies have suggested that HERCEPTIN® acts by triggering G1 cell cycle arrest.

Presently ErbB-directed therapeutics do not meet the current medical needs. ErbB-directed therapeutics have had only modest anti-tumor efficacy and are not as potent as anticipated from preclinical models. In most patients who initially respond to HERCEPTIN®, disease progression is noted within 1 year. In the metastatic setting, a median duration of roughly nine months was reported, at which point it appears that patients frequently become refractory to therapy. Studies have suggested that more complete blockade of the ErbB receptor family would be beneficial. As there are multiple functional domains of HER2, agents targeted to each of the domains could be a potentially valuable therapeutic. Additionally, there are harmful side effects of HERCEPTIN® treatment. Cardiac dysfunction, quantitated as a decrease in left ventricular ejection fraction (LVEF) of 10% from baseline or less than 50% total, was identified in roughly 7.1% of patients receiving HERCEPTIN® for 1 year versus 2.2% in patients randomized to observation in the HERA trial. Rates of severe and symptomatic congestive heart failure (CHF) were also significantly higher in the group randomized to HERCEPTIN®. Potentially, agents targeting a different HER2 epitopes could avoid these side effects. Accordingly, there remains an urgent need for agents targeting HER2.

The EGFR family of receptor tyrosine kinases are important regulators of cell growth and proliferation. One member of the family, ErbB2, has been implicated in a host of disorders and diseases including many forms of cancer.

Accordingly, there is an urgent need for therapeutic and diagnostic agents for detecting and treating ErbB2-mediated disorders including proliferative disorders.

### SUMMARY OF THE INVENTION

The invention relates to novel ErbB2 binding proteins that bind the extracellular domain (ECD) of ErbB2, in particular, human ErbB2. The novel binding protein can be antibody, an antigen-binding fragment of an antibody or a small modular immunopharmaceutical (SMIP). In various embodiments, the binding proteins: bind the ECD in the L1, CR1, L2 or CR2 domain, are ErbB2 agonists, increase tyrosine phosphorylation of ErbB2 and/or of AKT, MAP kinase (MAPK) or ERK 1/2, preferentially bind ErbB2 ECD homodimer over monomer or shed ECD, reduces ErbB2 mediated proliferation of cancer cells, increase apoptosis in cancer cells, increase the number of cells in S phase after treatment with the binding protein and reduce tumor growth in vivo, or any combination of these properties.

The invention further relates to nucleic acids encoding the binding proteins or their components, vectors and host cells comprising the nucleic acids and methods of producing the binding proteins by expressing them in the host cells.

In a further aspect, the invention provides kits and compositions comprising one or more binding proteins of the invention and in some embodiments, further comprising an additional component that is a therapeutic or diagnostic agent, particularly a chemotherapeutic agent.

The invention also provides methods for producing and identifying binding proteins of the invention and methods for using them, including for treating cancer or other ErbB2 mediated disorders in a subject in need thereof, for reducing proliferation of and/or increasing apoptosis in ErbB2 expressing cells, including cancer cells, for reducing tumor growth and for diagnostic uses, including detecting and/or quantifying the presence of ErbB2 or cells expressing it.

### BRIEF DESCRIPTION OF THE FIGURES

FIG. 1. Schematic representation of the selection strategy used in the generation of human anti-Her2 scFv binding domains.

FIG. 2 (A-M). Alignments of the heavy chain amino acid sequences of human anti-Her2 scFvs with the germline human V_{H} gene sequence. CDRs are in bold type.

FIG. 3 (A-L). Alignments of the light chain amino acid sequences of human anti-Her2 scFvs with the germline human V_{κ} or V_{λ} sequence. CDRs are in bold type.

FIG. 4. (A) Schematic diagram of the protein constructs used for selection and screening of scFvs and SMIPs that bind to the extracellular domain of Her2. (B) scFvs and SMIPs are binned into 4 distinct groups according to their binding phenotype as determined using the reagents in FIG. 4A. (* Herceptin contact sites)

FIG. 5. ELISA data for scFv binding to Her2. Binding data for phage-expressed scFv binding to Her2-expressing cells is shown on the left side of the table and data for soluble scFv binding to purified Her2 proteins is shown on the right. ELISA data is scored using a range that correlates with binding signal as indicated by −, + etc.

FIG. 6. Binding of HER2 SMIPs (HER067 and HER030), HERCEPTIN® (trastuzumab), and a trastuzumab SMIP (HER018) to (A) HER2 dimer; (B) HER2 monomer; and (C) HER2 shed ectodomain found in SKBR3 supernatant.

FIG. 7. ELISA and BIACORE® data for HERCEPTIN® (trastuzumab) and SMIPs binding to Her2. Graphs represent binding of HERCEPTIN® (trastuzumab), Her033 or Her030 binding to various Her2 proteins determined by standard ELISA methods. The table represents Kd values for HERCEPTIN® (trastuzumab), Her033, Her030 and Her018 (Herceptin SMIP) binding to various Her2 proteins as detected by BIACORE®.

FIG. 8 provides a summary of various specific SMIPs, HERCEPTIN® (trastuzumab), and a trastuzumab SMIP (HER018) binding to various HER2 molecules (different sizes and different species, including human, murine, and macaque) as well as binding to several different cancer cell lines.

FIGS. 9A-9H show cell surface binding of HER2SMIPs (HER067 and HER094), HERCEPTIN® (trastuzumab), and a trastuzumab SMIP (HER018) to cell lines (A) Ramos (Her2⁻/CD20⁺ control); (B) BT474; (C) 22rv1; (D) MDA-MB-175; (E) MDA-MB-361 (ATCC); (F) MDA-MB-453; (G) MDA-MB-361 (JL); and (H) SKBR3.

FIG. 10 provides a summary of the anti-proliferative activity of HER033SMIP and HERCEPTIN® (trastuzumab) on several different cancer cell lines.

FIG. 11. Proliferation of MDA-MB-361 cells following treatment with HER030 or HER033. MDA-MB-361 (ATCC) breast cancer cells were plated in 96-well format and treated with 0-10 ug/ml anti-Her2 or control reagents for 72 hr. Cells were washed, fixed, and stained with DAPI. Stained nuclei were counted using Cellomics High Content assay measuring fluorescence at 360 nM.

FIG. 12 provides a summary of the anti-proliferative activity of various specific SMIPs, HERCEPTIN® (trastuzumab), and a trastuzumab SMIP (HER018) on several different cancer cell lines.

FIG. 13. Western blot analysis of effect of Her033 on Her2 receptor phosphorylation (Y1248) following 24 hr treatment of MDA-MB-361 breast cancer cells. Cells were treated in vitro with Her033, HERCEPTIN® (trastuzumab), or a small molecule Her2 kinase inhibitor for 24 hrs either alone or in the presence of heregulin (HRG1 10 ng/ml) activation of Her3. Protein lysates (50 ug/well) were size fractionated by SDS-PAGE, transferred to nitrocellulose and probed with anti-phospho-Her2(Y1248) antibody. Inhibition of the Her2 receptor kinase blocked the endogenous Her2 autophosphorylation at tyrosine 1248 relative to control. Treatment with Herceptin did not significantly modulate receptor phosphorylation whereas treatment with Her033 stimulated Her2 receptor phosphorylation. Western blots were subsequently reprobed with anti-Actin antibody as protein loading control.

FIG. 14. Her033 increases downstream phosphoprotein signal transduction in MDA-MB-361 and BT474 breast cancer cells. Cells were plated in 96-well format and treated with anti-Her2 reagents or Heregulin for 10 minutes. Cells were stained with either rabbit anti-pAKT, anti-pERK, anti-pS6K, or anti-p38MAPK antibodies and ALEXA594 labeled secondary antibody and cellular fluorescence quantified by high content (Cellomics) analysis. In both breast cancer cell lines, treatment with Her033 SMIP induces phosphorylation of AKT and ERK proteins similar to treatment with the Her3 ligand Heregulin. MDA-MB-361 cells also demonstrate significant activation of p38MAP kinase.

FIG. 15. Kinetic analysis of Her033 stimulated downstream effector phosphorylation in MDA-MB-361 breast cancer cells. Cells were grown in 96-well format and treated with either anti-Her2 reagents or Her3 ligand Heregulin for 10 min to 24 hr as indicated. Cells were stained with either rabbit anti-pAKT, anti-pERK, anti-pS6K, or anti-p38MAPK antibodies and ALEXA594 labeled secondary antibody and cellular fluorescence quantified by high content (Cellomics) analysis. Her033 treatment induces sustained activation of AKT, ERK and p38MAP kinase phosphorylation in this cell line similar in magnitude to levels following stimulation with 10 ng/ml Heregulin.

FIGS. 16A and 16B show level of phosphorylation of ErbB2, and ERK1/2 in MDA-MB-361 cells when treated with HER2SMIP HER067, HERCEPTIN® (trastuzumab), and a trastuzumab SMIP (HER018).

FIG. 17 shows the effect on cell cycle of HER033SMIP, HERCEPTIN® (trastuzumab), and heregulin on the SKBR3 and BT474 cell lines.

FIG. 18 shows the effect on cell cycle of HER033SMIP, HERCEPTIN® (trastuzumab), and heregulin on the MDA-MB-453 and MDA-MB-361 cell lines.

FIG. 19. MDA-MB-361 xenograft progression in irradiated nu/nu mice. Female nu/nu mice were exposed to 400 rads of total body irradiation. After three days, they were injected subcutaneously in the dorsal right flank with 1×10⁷ MDA-MB-361 cells in Matrigel. When the tumors had reached a mass of 0.1-0.25 g, animals were dosed with Herceptin, HER033, or vehicle (100 ug/mouse, intraperitoneally) on days 1, 4, 6, 8 and 11 (n=10 mice/treatment group). Tumors were measured, and calculated tumor volumes for individual mice are shown for animals treated with vehicle (A), Herceptin (B), or HER033 (C). Animals developing tumors larger than 2.5 g were sacrificed. The mean tumor volume±SEM are plotted in (D). Means were not calculated for treatment groups in which animals with large tumors had been sacrificed.

FIG. 20. MDA-MB-361 xenograft progression in Balb/c nude mice. Male Balb/c nude mice were injected subcutaneously in the dorsal right flank with 1×10⁷ MDA-MB-361 cells in Matrigel. When the tumors had reached a mass of 0.1-0.25 g, animals were dosed with HERCEPTIN® (trastuzumab), HER033, or vehicle (100 ug/mouse, intraperitoneally) on days 1, 4, 6, 8 and 11 (n=10 mice/treatment group). Tumors were measured, and calculated tumor volumes for individual mice are shown for animals treated with vehicle (A), HERCEPTIN® (trastuzumab) (B), or HER033 (C). Animals developing tumors larger than 2.5 g were sacrificed. The mean tumor volume±SEM are plotted in (D). Means were not calculated for treatment groups in which animals with large tumors had been sacrificed.

FIGS. 21 and 22 show the in vivo efficacy of HER2SMIP HER033/HER067 when used to treat SCID-Beige having a tumor xenograft of MDA-MB-361 cells and the in vitro anti-proliferative activity on MDA-MB-361 cells. The top panel of FIG. 21 shows the mean tumor volume in mice treated with HER033 SMIP, HERCEPTIN® (trastuzumab), or vehicle (IgG) after 21 days. The bottom panel of FIG. 21 shows a titration of anti-proliferative activity of HER2SMIPs (HER067 and HER094) and trastuzumab SMIP (HER018) on the MDA-MB-361 cells used for xenografting in the mice. FIG. 22 shows the tumor volume of individual mice in each treatment group.

### DETAILED DESCRIPTION OF THE INVENTION

### I. Definitions

In order that the present invention may be more readily understood, certain terms are first defined. Additional definitions are set forth throughout the detailed description. The present invention provides novel binding proteins that, specifically bind the extra cellular domain (ECD) of ErbB2, especially human ErbB2. In some embodiments, the binding protein is an antibody or an antigen binding fragment of such antibody that specifically binds the ECD. In other embodiments, the binding protein is a small modular immunopharmaceutical (SMIP).

The term “antibody” refers to an intact four-chain molecule having 2 heavy chains and 2 light chains, each heavy chain and light chain having a variable domain and a constant domain, or an antigen-binding fragment thereof, and encompasses any antigen-binding domain. In various embodiments, an antibody of the invention may be polyclonal, monoclonal, monospecific, polyspecific, bi-specific, humanized, human, chimeric, synthetic, recombinant, hybrid, mutated, grafted (including CDR grafted), or an in vitro generated antibody.

The term “antigen-binding fragment” of an antibody that specifically binds the ECD of ErbB2 refers to a portion or portions of the antibody that specifically binds to the ECD. An antigen-binding fragment may comprise all or a portion of an antibody light chain variable region (V_{L}) and/or all or a portion of an antibody heavy chain variable region (V_{H}) so long as the portion or portions are antigen-binding. However, it does not have to comprise both. Fd fragments, for example, have two V_{H} regions and often retain some antigen-binding function of the intact antigen-binding domain. Examples of antigen-binding fragments of an antibody include (1) a Fab fragment, a monovalent fragment having the V_{L}, V_{H}, C_{L} and C_{H}1 domains; (2) a F(ab′)₂ fragment, a bivalent fragment having two Fab fragments linked by a disulfide bridge at the hinge region; (3) a Fd fragment having the two V_{H} and C_{H}1 domains; (4) a Fv fragment having the V_{L} and V_{H} domains of a single arm of an antibody, (5) a dAb fragment (Ward et al., (1989) Nature 341:544-546), that has a V_{H} domain; (6) an isolated complementarity determining region (CDR), and (7) a single chain Fv (scFv). Although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). These antibody fragments are obtained using conventional techniques known to those with skill in the art, and the fragments are evaluated for function in the same manner as are intact antibodies.

The term “effective amount” refers to a dosage or amount that is sufficient to alter ErbB2 activity, to ameliorate clinical symptoms or achieve a desired biological outcome, e.g., decreased cell growth or proliferation, decreased heterodimerization with another member of the EGF family decreased homodimerization, decrease tumor growth rate or tumor size, increased cell death etc.

The term “human antibody” includes antibodies having variable and constant region sequences corresponding substantially to human germline immunoglobulin sequences known in the art, including, for example, those described by Kabat et al. (See Kabat, et al. (1991) Sequences of Proteins of Immunological Interest, Fifth Edition, U.S. Department of Health and Human Services, NIH Publication No. 91-3242). The amino acid sequences of a human antibody, when aligned with germline immunoglobulin sequences, most closely align with human immunoglobulin sequences. The human antibodies of the invention may include amino acid residues not encoded by human germline immunoglobulin sequences (e.g., mutations introduced by random or site-specific mutagenesis in vitro or by somatic mutation in vivo). Such non-germline residues may occur in a framework region, a CDR, for example in the CDR3, or in the constant region. A human, antibody can have one or more residues, such as any number from 1-15, including all of the integers between 1 and 15, or more, replaced with an amino acid residue that is not encoded by the human germline immunoglobulin sequence. CDRs are as defined by Kabat or in Chothia C, Lesk AM, Canonical structures for the hypervariable regions of immunoglobulins, J Mol. Biol. 1987 Aug. 20; 196(4):901-17.

The phrase “inhibit” or “antagonize” an ErbB2/HER2 activity refers to a reduction, inhibition, or otherwise diminution of at least one activity of ErbB2 due to binding an anti-ErbB2 antibody or antigen binding portion, wherein the reduction is relative to the activity of ErbB2 in the absence of the same antibody or antigen-binding portion. The activity can be measured using any technique known in the art, including, for example, as described in the Examples. Activation of the Her2 receptor tyrosine kinase can be measured by the degree of phosphorylation of key tyrosine residues in the intracellular domain. For example, Tyr1248 is a known site of autophosphorylation and thus is a direct measure of Her2 receptor kinase activity. Typically the degree of phosphorylation can be determined by Western blot analysis probing with anti-phopho-Her2 specific antibodies (eg. Tyr1248, Tyr1139, Tyr1112, Tyr877, Tyr1221/1222). Alternatively, cells can be permeabilized and probed with fluorescently labeled phospho-Her2 antibodies and measured either by flow cytometry or high content (Cellomics) analysis. Additionally, the Her2 receptor can be immunoprecipitated, digested with trypsin protease and the degree of phosphorylation at specific sites within the individual Her2 peptides determined by standard Mass Spec techniques. Inhibition or antagonism does not necessarily indicate a total elimination of the ErbB2 polypeptide biological activity. In some embodiments, the reduction in activity may be about 10%, 20%, 30%, 40%, 50%, 60%, 70%, 75%, 80%, 85%, 90%, 95% or more, including 100% reduction, i.e., elimination of the activity.

The term “ErbB2” refers to erythroblastic leukemia viral oncogene homolog 2. In the case of human ErbB2, it also is known as c-erb-B2 or HER2/neu. In some embodiments the ErbB2 may comprise: (1) an amino acid sequence of a naturally occurring mammalian ErbB2 polypeptide (full length or mature form) or a fragment thereof, or a fragment thereof; (2) an amino acid sequence substantially identical to, e.g., at least 85%, 90%, 95%, 96%, 97%, 98%, 99% identical to said amino acid sequence or a fragment thereof; (3) an amino acid sequence that is encoded by a naturally occurring mammalian ErbB2 nucleotide sequence or a fragment thereof, or (4) a nucleotide sequence that hybridizes to the foregoing nucleotide sequence under stringent conditions, e.g., highly stringent conditions.

HER2 or c-erb-B2 encodes a transmembrane receptor protein of 185 kDa, which is structurally related to the epidermal growth factor receptor1. HER2 protein overexpression is observed in 25%-30% of primary breast cancers and is associated with decreased overall survival and a lowered response to chemotherapy and hormonal therapy, which can continue throughout the course of the disease and drives aggressive tumor growth.

The term “ErbB2 activity” refers to at least one cellular process initiated or interrupted as a result of ErbB2 binding to a receptor complex comprising ErbB2 and an ErbB receptor family member including ErbB1 (EGFR), ErbB2, ErbB3, ErbB4 or comprising an ErbB ligand such as but not limited to EGF, TGF-alpha, amphiregulin, betacellulin, heparin-binding EGF-like growth factor, GP30 on the cell. ErbB2 activity can be determined using any suitable assay methods, for example, protein overexpression can be determined using immunohistochemistry (IHC) and may also be inferred when HER2 gene amplification is identified using fluorescence in situ hybridization (FISH).

As used herein, “in vitro generated antibody” refers to an antibody where all or part of the variable region (e.g., at least one CDR) is generated in a non-immune cell selection (e.g., an in vitro phage display, protein chip or any other method in which candidate sequences can be tested for their ability to bind to an antigen). This term excludes sequences generated by genomic rearrangement in an immune cell.

The term “isolated” refers to a molecule that is substantially free of its natural environment. For instance, an isolated protein is substantially free of cellular material or other proteins from the cell or tissue source from which it was derived. The term also refers to preparations where the isolated protein is sufficiently pure for pharmaceutical compositions; or at least 70-80% (w/w) pure; or at least 80-90% (w/w) pure; or at least 90-95% pure; or at least 95%, 96%, 97%, 98%, 99%, or 100% (w/w) pure.

The phrase “percent identical” or “percent identity” refers to the similarity between at least two different sequences. This percent identity can be determined by standard alignment algorithms, for example, the Basic Local Alignment Tool (BLAST) described by Altshul et al. ((1990) J. Mol. Biol., 215: 403-410); the algorithm of Needleman et al. ((1970) J. Mol. Biol., 48: 444-453); or the algorithm of Meyers et al. ((1988) Comput. Appl. Biosci., 4: 11-17). A set of parameters may be the Blosum 62 scoring matrix with a gap penalty of 12, a gap extend penalty of 4, and a frameshift gap penalty of 5. The percent identity between two amino acid or nucleotide sequences can also be determined using the algorithm of E. Meyers and W. Miller ((1989) CABIOS, 4:11-17) that has been incorporated into the ALIGN program (version 2.0), using a PAM120 weight residue table, a gap length penalty of 12 and a gap penalty of 4. The percent identity is usually calculated by comparing sequences of similar length.

The terms “specific binding” or “specifically binds” refer to forming a complex that is relatively stable under physiologic conditions. Specific binding is characterized by a high affinity and a low to moderate capacity as distinguished from nonspecific binding which usually has a low affinity with a moderate to high capacity. Typically, binding is considered specific when the association constant K_{A} is higher than 10⁶M⁻¹. The appropriate binding conditions, such as concentration of antibodies, ionic strength of the solution, temperature, time allowed for binding, concentration of a blocking agent (e.g., serum albumin, milk casein), etc., may be optimized by a skilled artisan using routine techniques. An antibody is said to specifically bind an antigen when the K_{D} is ≦1 mM, preferably ≦100 nM.

As used herein, the term “stringent” describes conditions for hybridization and washing. Stringent conditions are known to those skilled in the art and can be found in Current Protocols in Molecular Biology, John Wiley & Sons, N.Y. (1989), 6.3.1-6.3.6. Aqueous and nonaqueous methods are described in that reference and either can be used. One example of stringent hybridization conditions is hybridization in 6× sodium chloride/sodium citrate (SSC) at about 45° C., followed by at least one wash in 0.2×SSC, 0.1% SDS at 50° C. A second example of stringent hybridization conditions is hybridization in 6×SSC at about 45° C., followed by at least one wash in 0.2×SSC, 0.1% SDS at 55° C. Another example of stringent hybridization conditions is hybridization in 6×SSC at about 45° C., followed by at least one wash in 0.2×SSC, 0.1% SDS at 60° C. A further example of stringent hybridization conditions is hybridization in 6×SSC at about 45° C., followed by at least one wash in 0.2×SSC, 0.1% SDS at 65° C. High stringent conditions include hybridization in 0.5M sodium phosphate, 7% SDS at 65° C., followed by at least one wash at 0.2×SSC, 1% SDS at 65° C.

The phrase “substantially as set out,” “substantially identical” or “substantially homologous” means that the relevant amino acid or nucleotide sequence (e.g., CDR(s), V_{H}, or V_{L} domain) will be identical to or have insubstantial differences (through conserved amino acid substitutions) in comparison to the sequences that are set out. Insubstantial differences include minor amino acid changes, such as 1 or 2 substitutions in a 5 amino acid sequence of a specified region. In the case of antibodies, the second antibody has the same specificity and has at least 50% of the affinity of the first antibody.

Sequences substantially identical or homologous (e.g., at least about 85% sequence identity) to the sequences disclosed herein are also part of this application. In some embodiment, the sequence identity can be about 85%, 90%, 95%, 96%, 97%, 98%, 99% or higher. Alternatively, substantial identity or homology exists when the nucleic acid segments will hybridize under selective hybridization conditions (e.g., highly stringent hybridization conditions), to the complement of the strand. The nucleic acids may be present in whole cells, in a cell lysate, or in a partially purified or substantially pure form.

The term “therapeutic agent” is a substance that treats or assists in treating a medical disorder. Therapeutic agents may include, but are not limited to, anti-proliferative agents, anti-cancer agents including chemotherapeutics, anti-virals, anti-infectives, immune modulators, and the like that modulate immune cells or immune responses in a manner that complements the ErbB2 activity of an anti-ErbB2 binding protein of the invention. Non-limiting examples and uses of therapeutic agents are described herein.

As used herein, a “therapeutically effective amount” of an anti-ErbB2 binding protein refers to an amount of an binding protein that is effective, upon single or multiple dose administration to a subject (such as a human patient) at treating, preventing, curing, delaying, reducing the severity of, and/or ameliorating at least one symptom of a disorder or recurring disorder, or prolonging the survival of the subject beyond that expected in the absence of such treatment.

The term “treatment” refers to a therapeutic or preventative measure. The treatment may be administered to a subject having a medical disorder or who ultimately may acquire the disorder, in order to prevent, cure, delay, reduce the severity of, and/or ameliorate one or more symptoms of a disorder or recurring disorder, or in order to prolong the survival of a subject beyond that expected in the absence of such treatment.

### II. Anti-ErbB2 Binding Proteins

In a first aspect, the invention provides novel ErbB2/HER2, particularly human ErbB2/HER2, ErbB2/HER2 binding proteins that bind in the extra-cellular domain (ECD). In various embodiments, the binding proteins of the invention bind in the LR1, CR1, LR2 or CR2 domain of the ECD. Unlike HERCEPTIN®, in some embodiments the binding proteins of the invention preferentially bind ErbB2 nomodimers over monomers or shed ECD. In some embodiments, the binding proteins of the invention bind ECD homodimers substantially more than monomers. In some cases, the binding protein has no appreciable or significant binding to ECD monomers or to shed ECD.

In some embodiments, the novel binding proteins are ErbB2 agonists and increase tyrosine phosphorylation of ErbB2, and at the same time, have anti-proliferative activity and pro-apoptotic activity.

The anti-ErbB2/HER2 binding proteins of the invention can be obtained by any of numerous methods known to those skilled in the art. For example, antibodies can be produced using recombinant DNA methods (U.S. Pat. No. 4,816,567). Monoclonal antibodies may be produced by generation of hybridomas (see e.g., Kohler and Milstein (1975) Nature, 256: 495-499) in accordance with known methods. Hybridomas formed in this manner are then screened using standard methods, such as enzyme-linked immunosorbent assay (ELISA) and surface plasmon resonance (BIACORE™) analysis, to identify one or more hybridomas that produce an antibody that specifically binds with a specified antigen. Any form of the specified antigen may be used as the immunogen, e.g., recombinant antigen, naturally occurring forms, any variants or fragments thereof, as well as antigenic peptide thereof.

One exemplary method of making antibodies includes screening protein expression libraries, e.g., phage or ribosome display libraries. Phage display is described, for example, in Ladner et al., U.S. Pat. No. 5,223,409; Smith (1985) Science 228:1315-1317; Clackson et al. (1991) Nature, 352: 624-628; Marks et al. (1991) J. Ma Biol., 222: 581-597 WO 92/18619; WO 91/17271; WO 92/20791; WO 92/15679; WO 93/01288; WO 92/01047; WO 92/09690; and WO 90/02809.

In addition to the use of display libraries, the specified antigen can be used to immunize a non-human animal, e.g., a rodent, e.g., a mouse, hamster, or rat. In one embodiment, the non-human animal includes at least a part of a human immunoglobulin gene. For example, it is possible to engineer mouse strains deficient in mouse antibody production with large fragments of the human Ig loci. Using the hybridoma technology, antigen-specific monoclonal antibodies derived from the genes with the desired specificity may be produced and selected. See, e.g., XENOMOUSE™, Green et al. (1994) Nature Genetics 7:13-21, US 2003-0070185, WO 96/34096, published Oct. 31, 1996, and PCT Application No. PCT/US96/05928, filed Apr. 29, 1996.

The subunit structures, e.g., a C_{H}, V_{H}, C_{L}, V_{L}, CDR, FR, and three-dimensional configurations of different classes of immunoglobulins are well known in the art. For a review of the antibody structure, see Antibodies: A Laboratory Manual, Cold Spring Harbor Laboratory, eds. Harlow et al., 1988. One of skill in the art will recognize that a complete 4-chain immunoglobulin comprises active portions, e.g., a portion of the V_{H} or V_{L} domain or a CDR that binds to the antigen, i.e., an antigen-binding fragment, or, e.g., the portion of the C_{H} subunit that binds to and/or activates, e.g., an Fc receptor and/or complement. CDRs typically refer to regions that are hypervariable in sequence and/or form structurally defined loops, for example, Kabat CDRs are based on sequence variability, as described in Sequences of Proteins of Immunological Interest, US Department of Health and Human Services (1991), eds. Kabat at al, or alternatively, to the location of the hypervariable structural loops as described by Chothia. See, e.g., Chothia, D. at al. (1992) J. Mol. Biol. 227:799-817; and Tomlinson at al. (1995) EMBO J. 14:4628-4638. Still another standard is the AbM definition used by Oxford Molecular's AbM antibody modelling software, which defines the contact hypervariable regions based on crystal structure. See, generally, e.g., Protein Sequence and Structure Analysis of Antibody Variable Domains. In: Antibody Engineering Lab Manual (Ed.: Duebel, S, and Kontermann, R., Springer-Verlag, Heidelberg). Embodiments described with respect to Kabat CDRs can alternatively be implemented using similar described relationships with respect to Chothia hypervariable loops or to the AbM-defined loops.

In another embodiment, a monoclonal antibody is obtained from the non-human animal, and then modified, e.g., humanized, deimmunized, chimeric, may be produced using recombinant DNA techniques known in the art. A variety of approaches for making chimeric antibodies have been described. See e.g., Morrison at al., Proc. Natl. Acad. Sci. U.S.A. 81:6851, 1985; Takeda et al., Nature 314:452, 1985, Cabilly et al., U.S. Pat. No. 4,816,567; Boss et al., U.S. Pat. No. 4,816,397; Tanaguchi et al., European Patent Publication EP171496; European Patent Publication 0173494, United Kingdom Patent GB 2177096B. Humanized antibodies may also be produced, for example, using transgenic mice that express human heavy and light chain genes, but are incapable of expressing the endogenous mouse immunoglobulin heavy and light chain genes. Winter describes an exemplary CDR-grafting method that may be used to prepare the humanized antibodies described herein (U.S. Pat. No. 5,225,539). All of the CDRs of a particular human antibody may be replaced with at least a portion of a non-human CDR, or only some of the CDRs may be replaced with non-human CDRs. It is only necessary to replace the number of CDRs required for binding of the humanized antibody to a predetermined antigen.

Humanized antibodies or fragments thereof can be generated by replacing sequences of the Fv variable domain that are not directly involved in antigen binding with equivalent sequences from human Fv variable domains. Exemplary methods for generating humanized antibodies or fragments thereof are provided by Morrison (1985) Science 229:1202-1207; by Oi et al. (1986) BioTechniques 4:214; and by U.S. Pat. No. 5,585,089; U.S. Pat. No. 5,693,761; U.S. Pat. No. 5,693,762; U.S. Pat. No. 5,859,205; and U.S. Pat. No. 6,407,213. Those methods include isolating, manipulating, and expressing the nucleic acid sequences that encode all or part of immunoglobulin Fv variable domains from at least one of a heavy or light chain. Such nucleic acids may be obtained from a hybridoma producing an antibody against a predetermined target, as described above, as well as from other sources. The recombinant DNA encoding the humanized antibody molecule can then be cloned into an appropriate expression vector.

In certain embodiments, a humanized antibody is optimized by the introduction of conservative substitutions, consensus sequence substitutions, germline substitutions and/or backmutations. Such altered immunoglobulin molecules can be made by any of several techniques known in the art, (e.g., Teng et al., Proc. Natl. Acad. Sci. U.S.A., 80: 7308-7312, 1983; Kozbor et al., Immunology Today, 4: 7279, 1983; Olson et al., Meth. Enzymol., 92: 3-16, 1982), and may be made according to the teachings of PCT Publication WO92/06193 or EP 0239400).

An antibody or fragment thereof may also be modified by specific deletion of human T cell epitopes or “deimmunization” by the methods disclosed in WO 98/52976 and WO 00/34317. Briefly, the heavy and light chain variable domains of an antibody can be analyzed for peptides that bind to MHC Class II; these peptides represent potential T-cell epitopes (as defined in WO 98/52976 and WO 00/34317). For detection of potential T-cell epitopes, a computer modeling approach termed “peptide threading” can be applied, and in addition a database of human MHC class II binding peptides can be searched for motifs present in the V_{H} and V_{L} sequences, as described in WO 98/52976 and WO 00/34317. These motifs bind to any of the 18 major MHC class II DR allotypes, and thus constitute potential T cell epitopes. Potential T-cell epitopes detected can be eliminated by substituting small numbers of amino acid residues in the variable domains, or preferably, by single amino acid substitutions. Typically, conservative substitutions are made. Often, but not exclusively, an amino acid common to a position in human germline antibody sequences may be used. Human germline sequences, e.g., are disclosed in Tomlinson, at al. (1992) J. Mol. Biol. 227:776-798; Cook, G. P. et al. (1995) Immunol. Today Vol. 16 (5): 237-242; Chothia, D. et al. (1992) J. Mol. Biol. 227:799-817; and Tomlinson et al. (1995) EMBO J. 14:4628-4638. The V BASE directory provides a comprehensive directory of human immunoglobulin variable region sequences (compiled by Tomlinson, I. A. et al., MRC Centre for Protein Engineering, Cambridge, UK). These sequences can be used as a source of human sequence, e.g., for framework regions and CDRs. Consensus human framework regions can also be used, e.g., as described in U.S. Pat. No. 6,300,064.

In certain embodiments, an antibody can contain an altered immunoglobulin constant or Fc region. For example, an antibody produced in accordance with the teachings herein may bind more strongly or with more specificity to effector molecules such as complement and/or Fc receptors, which can control several immune functions of the antibody such as effector cell activity, lysis, complement-mediated activity, antibody clearance, and antibody half-life. Typical Fc receptors that bind to an Fc region of an antibody (e.g., an IgG antibody) include, but are not limited to, receptors of the FcγRI, FcγRII, and FcγRIII and FcRn subclasses, including allelic variants and alternatively spliced forms of these receptors. Fc receptors are reviewed in Ravetch and Kinet, Annu. Rev. Immunol 9:457-92, 1991; Capel et al, Immunomethods 4:25-34, 1994; and de Haas et al., J. Lab. Clin. Med. 126:330-41, 1995).

For additional antibody production techniques, see Antibodies: A Laboratory Manual, eds. Harlow et al., Cold Spring Harbor Laboratory, 1988. The present invention is not necessarily limited to any particular source, method of production, or other special characteristics of an antibody.

In some embodiments, an anti-ErbB2 antibody of the invention may be a V_{HH} molecule. V_{HH} molecules (or nanobodies), as known to the skilled artisan, are heavy chain variable domains derived from immunoglobulins naturally devoid of light chains, such as those derived from Camelidae as described in WO9404678, incorporated herein by reference. Such a V_{HH} molecule can be derived from antibodies raised in Camelidae species, for example in camel, llama, dromedary, alpaca and guanaco and is sometimes called a camelid or camelized variable domain. See e.g., Muyldermans., J. Biotechnology (2001) 74(4):277-302, incorporated herein by reference. Other species besides Camelidae may produce heavy chain antibodies naturally devoid of light chain. V_{HH} molecules are about 10 times smaller than IgG molecules. They are single polypeptides in which the CDR3 is longer than a conventional antibody, the VH:VL interface residues are different, and extra cysteines are generally present. These molecules tend to be very stable, resisting extreme pH and temperature conditions. Moreover, they are resistant to the action of proteases which is not the case for conventional antibodies. Furthermore, in vitro expression of V_{HH}s produces high yield, properly folded functional V_{HH}s. In addition, antibodies generated in Camelids will recognize epitopes other than those recognized by antibodies generated in vitro through the use of antibody libraries or via immunization of mammals other than Camelids (see WO 9749805, that is incorporated herein by reference). In additional embodiments, an anti-ErbB2 antibodies or binding fragments of the invention may include single domain antibodies such as immunoglobulin new antigen receptors (IgNARs), which are a unique group of antibody isotypes found in the serum of sharks (Greenberg et al., Nature 374: 168-173 (1995); Nuttall et al., Mol. Immunol., 38: 313-326. (2001)). These are bivalent molecules, targeting antigen through a single immunoglobulin variable domain (˜13 kDa) displaying two complementarity determining region (CDR) loops (Roux et al., Proc. Natl. Acad. Sci., 95: 11804-11809 (1998)) and having unusually long and structurally complex CDR3s, which display a high degree of variability (Greenberg et al., 1995).

Antibodies, also known as immunoglobulins, are typically tetrameric glycosylated proteins composed of two light (L) chains of approximately 25 kDa each and two heavy (H) chains of approximately 50 kDa each. Two types of light chain, termed lambda and kappa, may be found in antibodies. Depending on the amino acid sequence of the constant domain of heavy chains, immunoglobulins can be assigned to five major classes: A, D, E, G, and M, and several of these may be further divided into subclasses (isotypes), e.g., IgG1, IgG2, IgG3, IgG4, IgA1, and IgA2. Each light chain includes an N terminal variable (V) domain (V_{L}) and a constant (C) domain (C_{L}). Each heavy chain includes an N terminal V domain (V_{H}), three or four C domains (C_{H}s), and a hinge region collectively referred to as the constant region of the heavy chain. The C_{H} domain most proximal to V_{H} is designated as C_{H}1. The V_{H} and V_{L} domains consist of four regions of relatively conserved sequences called framework regions (FR1, FR2, FR3, and FR4), that form a scaffold for three regions of hypervariable sequences also referred to as complementarity determining regions CDRs. CDRs are referred to as CDR1, CDR2, and CDR3. Accordingly, CDR constituents on the heavy chain may be referred to as HCDR1, HCDR2, and HCDR3, while CDR constituents on the light chain are referred to as LCDR1, LCDR2, and LCDR3. CDR3 is typically the greatest source of molecular diversity within the antibody-binding site.

The anti-ErbB2 binding proteins of the invention include complete 4-chain antibodies and antigen-binding fragments of complete antibodies. An antigen-binding fragment (also referred to as an antigen-binding portion) includes but is not limited to Fab, Fv and ScFv molecules. The Fab fragment (Fragment antigen-binding) consists of V_{H}—C_{H}1 and V_{L}—C_{L} domains covalently linked by a disulfide bond between the constant regions. The Fᵥ fragment is smaller and consists of V_{H} and V_{L} domains non-covalently linked. To overcome the tendency of non-covalently linked domains to dissociate, a single chain Fᵥ fragment (scFᵥ) can be constructed. The scFᵥ contains a flexible polypeptide that links (1) the C-terminus of V_{H} to the N-terminus of V_{L}, or (2) the C-terminus of V_{L} to the N-terminus of V_{H}. Repeating units of (Gly₄Ser) often 3 or 4 repeats may be used as a linker, but other linkers are known in the art.

A “bispecific” or “bifunctional antibody” is an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992). In one embodiment, the bispecific antibody comprises a first binding domain polypeptide, such as a Fab′ fragment, linked via an immunoglobulin constant region to a second binding domain polypeptide.

In some embodiments, an anti-ErbB2 binding protein of the invention is a Small Modular ImmunoPharmaceuticals (SMIP™). SMIPs and their uses and applications are disclosed in, e.g., U.S. Published Patent Application. Nos. 2003/0118592, 2003/0133939, 2004/0058445, 2005/0136049, 2005/0175614, 2005/0180970, 2005/0186216, 2005/0202012, 2005/0202023, 2005/0202028, 2005/0202534, and 2005/0238646, and related patent family members thereof, all of which are hereby incorporated by reference herein in their entireties.

A SMIP™ typically refers to a binding domain-immunoglobulin fusion protein that includes a binding domain polypeptide that is fused or otherwise connected to an immunoglobulin hinge or hinge-acting region polypeptide, which in turn is fused or otherwise connected to a region comprising one or more native or engineered constant regions from an immunoglobulin heavy chain, other than C_{H}1, for example, the C_{H}2 and C_{H}3 regions of IgG and IgA, or the C_{H}3 and C_{H}4 regions of IgE (see e.g., U.S. 2005/0136049 by Ledbetter, J. et al., which is incorporated by reference, for a more complete description). The binding domain-immunoglobulin fusion protein can further include a region that includes a native or engineered immunoglobulin heavy chain C_{H}2 constant region polypeptide (or C_{H}3 in the case of a construct derived in whole or in part from IgE) that is fused or otherwise connected to the hinge region polypeptide and a native or engineered immunoglobulin heavy chain C_{H}3 constant region polypeptide (or C_{H}4 in the case of a construct derived in whole or in part from IgE) that is fused or otherwise connected to the C_{H}2 constant region polypeptide (or C_{H}3 in the case of a construct derived in whole or in part from IgE). Typically, such binding domain-immunoglobulin fusion proteins are capable of at least one immunological activity selected from the group consisting of antibody dependent cell-mediated cytotoxicity, complement fixation, and/or binding to a target, for example, a target antigen, such as human ErbB2.

The binding domain of a SMIP of the invention may contain a complete V_{H} and a complete V_{L} joined by linker antigen-binding portions of a V_{H} and/or V_{L} and may V2 or be linked in either orientation, i.e., V_{H}-linker-V_{L} or V_{L}-linker-V_{H}. Any suitable linker can be used in a SMIP of the invention and will be known to those of skill in the art. Exemplary linkers may be found, for example in WO 2007/146968 Tables 5 and 10-12 of which are incorporated by reference in their entirety. Likewise, any immunoglobulin hinge sequence or hinge-acting sequence may be used in a SMIP of the invention.

In some SMIP embodiments at least one of the immunoglobulin heavy chain constant region polypeptides (i.e., CH2, CH3 or CH4) is from a human immunoglobulin heavy chain. In various embodiments, the immunoglobulin heavy chain constant region polypeptides are of an isotype selected from human IgG and human IgA. In certain further embodiments of the above described SMIP, the linker polypeptide comprises at least one polypeptide having as an amino acid sequence (Gly₄, Ser) and in certain other embodiments the linker polypeptide comprises at least three repeats of said polypeptide. In certain embodiments the immunoglobulin hinge region polypeptide comprises a human IgA hinge region polypeptide.

An immunoglobulin hinge region polypeptide, as discussed above, includes any hinge peptide or polypeptide that occurs naturally, as an artificial peptide or as the result of genetic engineering and that is situated in an immunoglobulin heavy chain polypeptide between the amino acid residues responsible for forming intrachain immunoglobulin-domain disulfide bonds in CH1 and CH2 regions; hinge region polypeptides for use in the present invention may also include a mutated hinge region polypeptide. Accordingly, an immunoglobulin hinge region polypeptide may be derived from, or may be a portion or fragment of (i.e., one or more amino acids in peptide linkage, typically 5-65 amino acids, preferably 10-50, more preferably 15-35, still more preferably 18-32, still more preferably 20-30, still more preferably 21, 22, 23, 24, 25, 26, 27, 28 or 29 amino acids) an immunoglobulin polypeptide chain region classically regarded as having hinge function, as described above. But, a hinge region polypeptide for use in the instant invention need not be so restricted and may include amino acids situated (according to structural criteria for assigning a particular residue to a particular domain that may vary, as known in the art) in an adjoining immunoglobulin domain such as a CH1 domain or a CH2 domain, or in the case of certain artificially engineered immunoglobulin constructs, an immunoglobulin variable region domain.

Wild-type immunoglobulin hinge region polypeptides include any naturally occurring hinge region that is located between the constant region domains, CH1 and CH2, of an immunoglobulin. The wild-type immunoglobulin hinge region polypeptide is preferably a human immunoglobulin hinge region polypeptide, preferably comprising a hinge region from a human IgG immunoglobulin, and more preferably, a hinge region polypeptide from a human IgG1 isotype. As is known to the art, despite the tremendous overall diversity in immunoglobulin amino acid sequences, immunoglobulin primary structure exhibits a high degree of sequence conservation in particular portions of immunoglobulin polypeptide chains, notably with regard to the occurrence of cysteine residues which, by virtue of their sulfyhydryl groups, offer the potential for disulfide bond formation with other available sulfydryl groups. Accordingly, in the context of the present invention wild-type immunoglobulin hinge region polypeptides may be regarded as those that feature one or more highly conserved (e.g., prevalent in a population in a statistically significant manner) cysteine residues, and in certain preferred embodiments a mutated hinge region polypeptide may be selected that contains zero or one cysteine residue and that is derived from such a wild-type hinge region.

A mutated immunoglobulin hinge region polypeptide may comprise a hinge region that has its origin in an immunoglobulin of a species, of an immunoglobulin isotype or class, or of an immunoglobulin subclass that is different from that of the CH2 and CH3 domains. For instance, in certain embodiments of the invention, the SMIP may comprise a binding domain polypeptide that is fused to an immunoglobulin hinge region polypeptide comprising a wild-type human IgA hinge region polypeptide, or a mutated human IgA hinge region polypeptide that contains zero or only one cysteine residues, as described herein. Such a hinge region polypeptide may be fused to an immunoglobulin heavy chain CH2 region polypeptide from a different Ig isotype or class, for example an IgG subclass, which in certain preferred embodiments will be the IgG1 subclass.

In some embodiments, an anti-ErbB2 antibody of the invention is a V_{HH} molecule. V_{HH} molecules (or nanobodies), as known to the skilled artisan, are heavy chain variable domains derived from immunoglobulins naturally devoid of light chains, such as those derived from Camelidae as described in WO9404678, incorporated herein by reference. Such a V_{HH} molecule can be derived from antibodies raised in Camelidae species, for example in camel, llama, dromedary, alpaca and guanaco and is sometimes called a camelid or camelized variable domain. See e.g., Muyldermans., J. Biotechnology (2001) 74(4):277-302, incorporated herein by reference. Other species besides Camelidae may produce heavy chain antibodies naturally devoid of light chain. V_{HH} molecules are about 10 times smaller than IgG molecules. They are single polypeptides and very stable, resisting extreme pH and temperature conditions. Moreover, they are resistant to the action of proteases which is not the case for conventional antibodies. Furthermore, in vitro expression of V_{HH}S produces high yield, properly folded functional V_{HH}S. In addition, antibodies generated in Camelids will recognize epitopes other than those recognized by antibodies generated in vitro through the use of antibody libraries or via immunization of mammals other than Camelids (see WO 9749805, that is incorporated herein by reference).

Amino acid (AA) sequences of illustrative heavy chain variable domains (V_{H}) and light chain variable domains (V_{L}) of the anti-ErbB2 antibodies of this invention, are set forth in the attached Sequence Table. Table 1 provides the Sequence Identifiers (SEQ ID Nos) of the V_{H} and V_{L} domains. Thirty-one specific embodiments of the antibodies are identified as: S1R2A_CS_{—}1F7, S1R2A_CS_{—}1D11, S1R2C_CS_{—}1D3, S1R2C_CS_{—}1H12, S1R2A_CS_{—}1D3, S1R3B2_BMV_{—}1E1, S1R3C1_CS_{—}1D3, S1R3B2_DP47_{—}1E8, S1R3B2_BMV_{—}1G2, S1R3B2_BMV_{—}1H5, S1R3C1_CS_{—}1A6, S1R3B2_DP47_{—}1C9, S1R3B2_DP47_{—}1E10, S1R3C1_CS_{—}1B10, S1R3A1_BMV_{—}1F3, S1R3B1_BMV_{—}1G11, S1R3A1_BMV_{—}1G4, S1R3B1_BMV_{—}1H11, S1R3A1_CS_{—}1B9, S1R3B1_BMV_{—}1H9, S1R3A1_CS_{—}1B10, S1R3B1_BMV_{—}1C12, S1R3C1_BMV_{—}1H11, S1R3B1_BMV_{—}1A10, S1R3A1_CS_{—}1D11, S1R3C1_DP47_{—}1H1, S1R3A1_CS_{—}1B12, S1R3B1_BMV_{—}1H5, S1R3A1_DP47_{—}1A6, S1R3B1_DP47_{—}1E1 and S1R3B1_BMV_{—}1A1.

TABLE 1
HUMAN ANTI-ErbB2 BINDING DOMAINS

| | SEQUENCE IDENTIFIER (SEQ ID Nos:) | |
|---|---|---|
| | Variable Domain Protein Sequences | |
| scFv | Heavy | Light |
| S1R2A_CS_1F7 | 1 | 2 and 63 |
| S1R2A_CS_1D11 | 3 | 4 and 64 |
| S1R2C_CS_1D3 | 5 and 65 | 6 and 66 |
| S1R2C_CS_1H12 | 7 and 67 | 8 and 68 |
| S1R2A_CS_1D3 | 9 | 10 and 69 |
| S1R3B2_BMV_1E1 | 11 | 12 and 70 |
| S1R3C1_CS_1D3 | 13 | 14 and 71 |
| S1R3B2_DP47_1E8 | 15 | 16 and 72 |
| S1R3B2_BMV_1G2 | 17 | 18 and 73 |
| S1R3B2_BMV_1H5 | 19 | 20 and 74 |
| S1R3C1_CS_1A6 | 21 | 22 and 75 |
| S1R3B2_DP47_1C9 | 23 | 24 and 76 |
| S1R3B2_DP47_1E10 | 25 | 26 and 77 |
| S1R3C1_CS_1B10 | 27 | 28 and 78 |
| S1R3A1_BMV_1F3 | 29 | 30 and 79 |
| S1R3B1_BMV_1G11 | 31 | 32 and 80 |
| S1R3A1_BMV_1G4 | 33 | 34 and 81 |
| S1R3B1_BMV_1H11 | 35 | 36 and 82 |
| S1R3A1_CS_1B9 | 37 | 38 and 83 |
| S1R3B1_BMV_1H9 | 39 | 40 and 84 |
| S1R3A1_CS_1B10 | 41 | 42 and 85 |
| S1R3B1_BMV_1C12 | 43 | 44 and 86 |
| S1R3C1_BMV_1H11 | 45 | 46 and 87 |
| S1R3B1_BMV_1A10 | 47 | 48 and 88 |
| S1R3A1_CS_1D11 | 49 | 50 and 89 |
| S1R3C1_DP47_1H1 | 51 | 52 and 90 |
| S1R3A1_CS_1B12 | 53 | 54 and 91 |
| S1R3B1_BMV_1H5 | 55 | 56 and 92 |
| S1R3A1_DP47_1A6 | 57 | 58 and 93 |
| S1R3B1_DP47_1E1 | 59 | 60 and 94 |
| S1R3B1_BMV_1A1 | 61 | 62 and 95 |

According to the nomenclature used herein, “S1R2A_CS_{—}1F7” indicates clone 1F7 from round 2A of the first selection from the CS library.

An anti-ErbB2 binding protein of this invention may optionally comprise antibody constant regions or parts thereof. For example, a V_{L} domain may be attached at its C-terminal end to a light chain constant domain which can be a Cκ or a Cλ. Similarly, a V_{H} domain or portion thereof may be attached to all or part of a heavy chain constant region, which can be a IgA, IgD, IgE, IgG, or IgM constant region or any isotype subclass including IgG1, IgG2, IgG3, IgG4, IgA1 or IgA2. Constant region sequences are known in the art (see, for example, Kabat et al., Sequences of Proteins of Immunological Interest, No. 91-3242, National Institutes of Health Publications, Bethesda, Md. (1991)). Therefore, binding proteins within the scope of this invention may include V_{H} and V_{L} domains, or a portion thereof, combined with constant regions or portions thereof known in the art.

In certain embodiments of the invention, the ErbB2 binding protein comprises a V_{H} domain, a V_{L} domain, or a combination thereof, comprising the V_{H} or V_{L} amino acid sequence, respectively, found in any one of S1R2A_CS_{—}1F7, S1R2A_CS_{—}1D11, S1R2C_CS_{—}1D3, S1R2C_CS_{—}1H12, S1R2A_CS_{—}1D3, S1R3B2_BMV_{—}1E1, S1R3C1_CS_{—}1D3, S1R3B2_DP47_{—}1E8, S1R3B2_BMV_{—}1G2, S1R3B2_BMV_{—}1H5, S1R3C1_CS_{—}1A6, S1R3B2_DP47_{—}1C9, S1R3B2_DP47_{—}1E10, S1R3C1_CS_{—}1B10, S1R3A1_BMV_{—}1F3, S1R3B1_BMV_{—}1G11, S1R3A1_BMV_{—}1G4, S1R3B1_BMV_{—}1H11, S1R3A1_CS_{—}1B9, S1R3B1_BMV_{—}1H9, S1R3A1_CS_{—}1B10, S1R3B1_BMV_{—}1C12, S1R3C1_BMV_{—}1H11, S1R3B1_BMV_{—}1A10, S1R3A1_CS_{—}1D11, S1R3C1_DP47_{—}1H1, S1R3A1_CS_{—}1B12, S1R3B1_BMV_{—}1H5, S1R3A1_DP47_{—}1A6, S1R3B1_DP47_{—}1E1 and S1R3B1_BMV_{—}1A1. In some embodiments, the V_{H} and V_{L} are from the same reference antibody. That is, an anti-ErbB2 binding protein of the invention may comprise both the V_{H} and V_{L} amino acid sequence of one of the above-listed antibodies.

An anti-ErbB2 antibody of the invention may comprise one, two, three, four, five or all six complementarity determining regions (CDRs) from any one of the above-listed antibodies. In some embodiments, an anti-ErbB2 binding protein of the invention comprises the HCDR1, HCDR2 and HCDR3 (heavy chain CDR set), the LCDR1, LCDR2 and LCDR3 (light chain CDR set) or both the heavy chain CDR set and the light chain CDR set of one of the thirty-one antibodies exemplified herein.

A CDR3 sequence found in any one of the thirty-one specifically exemplified antibodies are encompassed within the scope of this invention. For example, in one embodiment, an anti-ErbB2 binding protein of the invention comprises an HCDR3 amino acid sequence found in any one of S1R2A_CS_{—}1F7, S1R2A_CS_{—}1D11, S1R2C_CS_{—}1D3, S1R2C_CS_{—}1H12, S1R2A_CS_{—}1D3, S1R3B2_BMV_{—}1E1, S1R3C1 CS_{—}1D3, S1R3B2_DP47_{—}1E8, S1R3B2_BMV_{—}1G2, S1R3B2_BMV_{—}1H5, S1R3C1_CS_{—}1A6, S1R3B2_DP47_{—}1C9, S1R3B2 DP47_{—}1E10, S1R3C1_CS_{—}1B10, S1R3A1_BMV_{—}1F3, S1R3B1_BMV_{—}1G11, S1R3A1_BMV_{—}1G4, S1R3B1_BMV_{—}1H11, S1R3A1_CS_{—}1B9, S1R3B1_BMV_{—}1H9, S1R3A1_CS_{—}1B10, S1R3B1_BMV_{—}1C12, S1R3C1_BMV_{—}1H11, S1R3B1_BMV_{—}1A10, S1R3A1_CS_{—}1D11, S1R3C1_DP47_{—}1H1, S1R3A1_CS_{—}1B12, S1R3B1_BMV_{—}1H5, S1R3A1_DP47_{—}1A6, S1R3B1_DP47_{—}1E1 or S1R3B1_BMV_{—}1A1.

In certain embodiments, the V_{H} and/or V_{L} domains may be germlined, i.e., the framework regions (FR) of these domains are mutated using conventional molecular biology techniques to match the germline sequence. In other embodiments, the FR sequences remain diverged from the consensus germline sequences.

In one embodiment, mutagenesis is used to make an antibody more similar to one or more germline sequences. This may be desirable when mutations are introduced into the framework region of an antibody through somatic mutagenesis or through error prone PCR. Germline sequences for the V_{H} and V_{L} domains can be identified by performing amino acid and nucleic acid sequence alignments against the VBASE database (MRC Center for Protein Engineering, UK). VBASE is a comprehensive directory of all human germline variable region sequences compiled from over a thousand published sequences, including those in the current releases of the Genbank and EMBL data libraries. In some embodiments, the FR regions of the scFvs are mutated in conformity with the closest matches in the VBASE database and the CDR portions are kept intact.

In certain embodiments, an anti-ErbB2 binding of this invention specifically binds the same epitope as, competes with or cross-competes with an antibody selected from the group consisting of: S1R2A_CS_{—}1F7, S1R2A_CS_{—}1D11, S1R2C_CS_{—}1D3, S1R2C_CS_{—}1H12, S1R2A_CS_{—}1D3, S1R3B2_BMV_{—}1E1, S1R3C1_CS_{—}1D3, S1R3B2_DP47_{—}1E8, S1R3B2_BMV_{—}1G2, S1R3B2_BMV_{—}1H5, S1R3C1_CS_{—}1A6, S1R3B2_DP47_{—}1C9, S1R3B2_DP47_{—}1E10, S1R3C1_CS_{—}1B10, S1R3A1_BMV_{—}1F3, S1R3B1_BMV_{—}1G11, S1R3A1_BMV_{—}1G4, S1R3B1_BMV_{—}1H11, S1R3A1_CS_{—}1B9, S1R3B1_BMV_{—}1H9, S1R3A1_CS_{—}1B10, S1R3B1_BMV_{—}1C12, S1R3C1_BMV_{—}1H11, S1R3B1_BMV_{—}1A10, S1R3A1_CS_{—}1D11, S1R3C1_DP47_{—}1H1, S1R3A1_CS_{—}1B12, S1R3B1_BMV_{—}1H5, S1R3A1_DP47_{—}1A6, S1R3B1_DP47_{—}1E1 and S1R3B1_BMV_{—}1A1, for binding to ErbB2. In some embodiments, such competing or ErbB2-mediated cross-competing binding protein is an ErbB2 agonist and may further reduce proliferation of a cancer call, reduce the rate of growth of an ErbB2-expressing tumor and/or increases apoptosis in such cells and tumors. In some embodiments, such competing or cross-competing binding proteins bind ErbB2 ECD homo-dimers but do not bind ECD monomers or shed ECD.

Such antibodies can be identified in a competitive binding assay.

One can determine whether an antibody binds to the same epitope or cross competes for binding with a binding protein of the invention antibody by using methods known in the art. In one embodiment, one allows the binding protein of the invention to bind to ErbB2 under saturating conditions and then measures the ability of the test protein to bind to the ECD. If the test antibody is able to bind to the ECD at the same time as the reference binding protein, then the test antibody binds to a different epitope than the reference binding protein. However, if the test protein is not able to bind the to the ECD at the same time, then the test protein binds to the same epitope, an overlapping epitope, or an epitope that is in close proximity to the epitope bound by the binding protein of the invention. This experiment can be performed using ELISA, RIA, BIACORE™, or flow cytometry. To test whether a binding protein cross-competes with another anti-ErbB2 binding protein, one may use the competition method described above in two directions, i.e. determining if the known binder blocks the test binder and vice versa. In a preferred embodiment, the experiment is performed using BIACORE™.

In one embodiment, the association constant (K_{A}) of an ErbB2 binding protein of the invention is at least 10⁶ M⁻¹. In another embodiment, the association constant of these antibodies for human ErbB2 is at least 10⁹ M. In other embodiments, the association constant of these antibodies for human ErbB2 is at least 10¹⁰ M⁻¹, at least 10¹¹ M⁻¹, or at least 10¹² M⁻¹. The binding affinity may be determined using techniques known in the art, such as ELISA, biosensor technology, such as biospecific interaction analysis, or other techniques including those described in this application.

In addition to sequence homology analyses, epitope mapping (see, e.g., Epitope Mapping Protocols, ed. Morris, Humana Press, 1996), and secondary and tertiary structure analyses can be carried out to identify specific 3D structures assumed by the presently disclosed antibodies and their complexes with antigens. Such methods include, but are not limited to, X-ray crystallography (Engstom (1974) Biochem. Exp. Biol., 11:7-13) and computer modeling of virtual representations of the present antibodies (Fletterick et al. (1986) Computer Graphics and Molecular Modeling, in Current Communications in Molecular Biology, Cold Spring Harbor Laboratory, Cold Spring Harbor, N.Y.).

The invention further provides anti-ErbB2 binding proteins that comprise altered V_{H} and/or V_{L} sequence(s) compared to the sequences in Table 1. Such binding proteins may be produced by a skilled artisan using techniques well-known in the art. For example, amino acid substitutions, deletions, or additions can be introduced in FR and/or CDR regions. FR changes are usually designed to improve the stability and immunogenicity of the antibody, while CDR changes are typically designed to increase antibody affinity for its antigen. The changes that increase affinity may be tested by altering CDR sequence and measuring antibody affinity for its target (see Antibody Engineering, 2nd ed., Oxford University Press, ed. Borrebaeck, 1995).

Antibodies whose CDR sequences differ insubstantially from those found in any one of thirty-one specifically exemplified antibodies are encompassed within the scope of this invention. Typically, this involves substitution of an amino acid with an amino acid having similar charge, hydrophobic, or stereochemical characteristics. More drastic substitutions in FR regions, in contrast to CDR regions, may also be made as long as they do not adversely affect (e.g., reduce affinity by more than 50% as compared to unsubstituted antibody) the binding properties of the binding protein. Substitutions may also be made to germline the binding protein or stabilize the antigen binding site.

Conservative modifications will produce molecules having functional and chemical characteristics similar to those of the molecule from which such modifications are made. In contrast, substantial modifications in the functional and/or chemical characteristics of the molecules may be accomplished by selecting substitutions in the amino acid sequence that differ significantly in their effect on maintaining (1) the structure of the molecular backbone in the area of the substitution, for example, as a sheet or helical conformation, (2) the charge or hydrophobicity of the molecule at the target site, or (3) the size of the molecule.

For example, a “conservative amino acid substitution” may involve a substitution of a native amino acid residue with a normative residue such that there is little or no effect on the polarity or charge of the amino acid residue at that position. (See, for example, MacLennan et at., 1998, Acta Physiol. Scand. Suppl. 643:55-67; Sasaki et al., 1998, Adv. Biophys. 35:1-24).

Desired amino acid substitutions (whether conservative or non-conservative) can be determined by those skilled in the art at the time such substitutions are desired. For example, amino acid substitutions can be used to identify important residues of the molecule sequence, or to increase or decrease the affinity of the molecules described herein. Exemplary amino acid substitutions include, but are not limited to, those set forth in Table 2.

TABLE 2
Amino Acid Substitutions

| | | More |
|---|---|---|
| Original | Exemplary | Conservative |
| Residues | Substitutions | Substitutions |
| Ala (A) | Val, Leu, Ile | Val |
| Arg (R) | Lys, Gln, Asn | Lys |
| Asn (N) | Gln | Gln |
| Asp (D) | Glu | Glu |
| Cys (C) | Ser, Ala | Ser |
| Gln (Q) | Asn | Asn |
| Gly (G) | Pro, Ala | Ala |
| His (H) | Asn, Gln, Lys, Arg | Arg |
| Ile (I) | Leu, Val, Met, Ala, Phe, | Leu |
| | Norleucine | |
| Leu (L) | Norleucine, Ile, Val, Met, | Ile |
| | Ala, Phe | |
| Lys (K) | Arg, 1,4 Diamino-butyric | Arg |
| | Acid, Gln, Asn | |
| Met (M) | Leu, Phe, Ile | Leu |
| Phe (F) | Leu, Val, Ile, Ala, Tyr | Leu |
| Pro (P) | Ala | Gly |
| Ser (S) | Thr, Ala, Cys | Thr |
| Thr (T) | Ser | Ser |
| Trp (W) | Tyr, Phe | Tyr |
| Tyr (Y) | Trp, Phe, Thr, Ser | Phe |
| Val (V) | Ile, Met, Leu, Phe, Ala, | Leu |
| | Norleucine | |

In certain embodiments, conservative amino acid substitutions also encompass non-naturally occurring amino acid residues that are typically incorporated by chemical peptide synthesis rather than by synthesis in biological systems.

In one embodiment, the method for making a variant V_{H} domain comprises adding, deleting, or substituting at least one amino acid in the disclosed V_{H} domains, and testing the variant V_{H} domain for ErbB2 binding or modulation of ErbB2 activity.

An analogous method for making a variant V_{L} domain comprises adding, deleting, or substituting at least one amino acid in the disclosed V_{L} domains, and testing the variant V_{L} domain for ErbB2 binding or modulation of ErbB2 activity.

A further aspect of the invention provides a method for preparing antibodies or antigen-binding fragments that specifically bind ErbB2. The method comprises:

(a) providing a starting repertoire of nucleic acids encoding a V_{H} domain that lacks at least one CDR or contains at least one CDR to be replaced;

(b) inserting into or replacing the CDR region of the starting repertoire with at least one donor nucleic acid encoding an amino acid sequence as substantially set out herein for a V_{H} CDR, yielding a product repertoire;

(c) expressing the nucleic acids of the product repertoire;

(d) selecting a specific antigen-binding fragment that binds to ErbB2; and

(e) recovering the specific antigen-binding fragment or nucleic acid encoding it.

In an analogous method, at least one V_{L} CDR of the invention is combined with a repertoire of nucleic acids encoding a V_{L} domain that lacks at least one CDR or contains at least one CDR to be replaced. The at least one V_{H} or V_{L}

CDR may be a CDR1, a CDR2, a CDR3, or a combination thereof, found in any of the thirty-one specifically exemplified antibodies.

In one embodiment, the variable domain includes a CDR3 to be replaced or lacks a CDR3 encoding region and the at least one donor nucleic acid encodes a CDR3 amino acid sequence found in any one of SEQ ID Nos:1-62 or substantially as found in such sequence.

In another embodiment, the variable domain includes a CDR1 to be replaced or lacks a CDR1 encoding region and the at least one donor nucleic acid encodes a CDR1 amino acid sequence found in any one of SEQ ID Nos: 1-62.

In another embodiment, the variable domain includes a CDR2 to be replaced or lacks a CDR2 encoding region and the at least one donor nucleic acid encodes a CDR2 amino acid sequence found in any one of SEQ ID Nos: 1-62.

In another embodiment, the variable domain includes a CDR3 to be replaced or lacks a CDR3 encoding region and further comprises a CDR1 to be replaced or lacks a CDR1 encoding region, where the at least one donor nucleic acid encodes a CDR3a CDR1 amino acid sequence, respectively, found in any one of SEQ ID Nos: 1-62.

In another embodiment, the variable domain includes a CDR3 to be replaced or lacks a CDR3 encoding region and further comprises a CDR2 to be replaced or lacks a CDR2 encoding region, where the at least one donor nucleic acid encodes a CDR3 or CDR2 amino acid sequence, respectively, found in any one of SEQ ID Nos: 1-62.

In another embodiment, the variable domain includes a CDR3 to be replaced or lacks a CDR3 encoding region and further comprises a CDR1 and a CDR2 to be replaced or lacks a CDR1 and a CDR2 encoding region, where the at least one donor nucleic acid encodes CDR3, CDR1 or CDR2 amino acid sequence, respectively, found in any one of SEQ ID Nos: 1-62.

Using recombinant DNA methodology, a disclosed CDR sequence may be introduced into a repertoire of V_{H} or V_{L} domains lacking the respective CDR (Marks et al. (BioTechnology (1992) 10: 779-783). For example, a primer adjacent to the 5′ end of the variable domain and a primer to the third FR can be used to generate a repertoire of variable domain sequences lacking CDR3. This repertoire can be combined with a CDR3 of an antibody disclosed herein. Using analogous techniques, portions of a disclosed CDR sequence may be shuffled with portions of CDR sequences from other antibodies to provide a repertoire of antigen-binding fragments that bind ErbB2. Either repertoire can be expressed in a host system such as phage display (described in WO 92/01047 and its corresponding U.S. Pat. No. 5,969,108) so suitable antigen-binding fragments that bind to ErbB2 can be selected.

A further alternative uses random mutagenesis of a V_{H} or V_{L} sequence disclosed herein to generate variant V_{H} or V_{L} domains still capable of binding ErbB2. A technique using error-prone PCR is described by Gram et al. (Proc. Nat. Acad. Sci. U.S.A. (1992) 89: 3576-3580).

Another method uses direct mutagenesis of a V_{H} or V_{L} sequence disclosed herein. Such techniques are described by Barbas et al. (Proc. Nat. Acad. Sci. U.S.A. (1994) 91: 3809-3813) and Schier et al. (J. Mol. Biol. (1996) 263: 551-567).

Also encompassed by the invention is a portion of a variable domain that comprises at least one CDR region substantially as set out herein and, optionally, intervening framework regions from the V_{H} or V_{L} domains as set out herein. Variable domains lacking a portion of the N-terminus of the FR1 and/or a portion of the C₁ terminus of the FR4 are also encompassed by the invention. Additional residues at the N-terminal of the FR1 or C-terminal of the FR4 of the variable domain may not be the same residues found in naturally occurring antibodies. For example, construction of antibodies by recombinant DNA techniques often introduces N- or C-terminal residues from its use of linkers. Some linkers may be used to join variable domains to other variable domains (e.g., diabodies), constant domains, or proteinaceous labels.

Although the embodiments specifically exemplified herein comprise a “matching” pair of V_{H} and V_{L} domains, a skilled artisan will recognize that alternative embodiments may comprise binding proteins containing only a single CDR from either V_{L} or V_{H} domain. Either one of the V_{H} domain or V_{L} domain can be used to screen for complementary domains capable of forming a two-domain specific binding protein capable of, binding to ErbB2 ECD. The screening may be accomplished by phage display screening methods using the so-called hierarchical dual combinatorial approach disclosed in WO 92/01047. In this approach, an individual colony containing either a H or L chain clone is used to infect a complete library of clones encoding the other chain (L or H), and the resulting two-chain specific antigen-binding domain is selected in accordance with phage display techniques as described.

In some alternative embodiments, the anti-ErbB2 binding protein can be linked to a protein (e.g., albumin) by chemical cross-linking or recombinant methods. The disclosed antibodies may also be linked to a variety of nonproteinaceous polymers (e.g., polyethylene glycol, polypropylene glycol, or polyoxyalkylenes) in manners set forth in U.S. Pat. No. 4,640,835; 4,496,689; 4,301,144; 4,670,417; 4,791,192; or 4,179,337. The binding proteins can be chemically modified by covalent conjugation to a polymer, for example, to increase their half-life in blood circulation. Exemplary polymers and attachment methods are shown in U.S. Pat. Nos. 4,766,106; 4,179,337; 4,495,285; and 4,609,546.

Binding proteins of the invention can be modified to alter their glycosylation; that is, at least one carbohydrate moiety can be deleted or added to the binding protein. Deletion or addition of glycosylation sites can be accomplished by changing amino acid sequence to delete or create glycosylation consensus sites, that are well known in the art. Another means of adding carbohydrate moieties is the chemical or enzymatic coupling of glycosides to amino acid residues of the antibody (see WO 87/05330 and Aplin et al. (1981) CRC Grit. Rev. Biochem., 22: 259-306).

Removal of carbohydrate moieties can also be accomplished chemically or enzymatically (see Hakimuddin et al. (1987) Arch. Biochem. Biophys., 259: 52; Edge et al. (1981) Anal. Biochem., 118: 131; Thotakura et al. (1987) Meth. Enzymol., 138: 350).

Methods for altering an antibody constant region are known in the art. Antibodies with altered function (e.g., altered affinity for an effector ligand such as FcR on a cell or the C1 component of complement) can be produced by replacing at least one amino acid residue in the constant portion of the antibody with a different residue (see e.g., EP 388,151 A1, U.S. Pat. No. 5,624,821 and U.S. Pat. No. 5,648,260). Similar types of alterations could be described that if applied to a murine or other species antibody would reduce or eliminate similar functions.

For example, it is possible to alter the affinity of an Fc region of an antibody (e.g., an IgG, such as a human IgG) for FcR (e.g., Fc gamma R1) or C1q. The affinity may be altered by replacing at least one specified residue with at least one residue having an appropriate functionality on its side chain, or by introducing a charged functional group, such as glutamate or aspartate, or perhaps an aromatic non-polar residue such as phenylalanine, tyrosine, tryptophan or alanine (see e.g., U.S. Pat. No. 5,624,821).

For example, replacing residue 297 (asparagine) with alanine in the IgG constant region significantly inhibits recruitment of effector cells, while only slightly reducing (about three fold weaker) affinity for Clq (see e.g., U.S. Pat. No. 5,624,821). The numbering of the residues in the heavy chain is that of the EU index (see Kabat et al., 1991 supra). This alteration destroys the glycosylation site and it is believed that the presence of carbohydrate is required for Fc receptor binding. Any other substitution at this site that destroys the glycosylation site is believed to cause a similar decrease in lytic activity. Other amino acid substitutions, e.g., changing any one of residues 318 (Glu), 320 (Lys) and 322 (Lys), to Ala, are also known to abolish Clq binding to the Fc region of IgG antibodies (see e.g., U.S. Pat. No. 5,624,821).

Modified binding proteins can be produced that have a reduced interaction with an Fc receptor. For example, it has been shown that in human IgG₃, which binds to the human Fc gamma R1 receptor, changing Leu 235 to Glu destroys its interaction with the receptor. Mutations on adjacent or close sites in the hinge link region of an antibody (e.g., replacing residues 234, 236 or 237 with Ala) can also be used to affect antibody affinity for the Fc gamma R1 receptor. The numbering of the residues in the heavy chain is based in the EU index (see Kabat et al., 1991 supra).

Additional methods for altering the lytic activity of an binding protein, for example, by altering at least one amino acid in the N-terminal region of the C_{H}2 domain, are described in WO 94/29351 by Morgan et al. and U.S. Pat. No. 5,624,821.

One of skill in the art will appreciate that the modifications described above are not all-exhaustive, and that many other modifications are obvious to a skilled artisan in light of the teachings of the present disclosure.

A binding protein of this invention may be tagged with a detectable or functional label. These labels include radiolabels (e.g., ¹³¹I or ⁹⁹Tc), enzymatic labels (e.g., horseradish peroxidase or alkaline phosphatase), and other chemical moieties (e.g., biotin).

In some embodiments, the invention features a human, monoclonal antibody that specifically binds the ECD, ErbB2, in particular, human ErbB2 and possesses one or more of the following characteristics: (1) it is an in vitro generated antibody (2) it is an in vivo generated antibody (e.g., transgenic mouse system); (3) it binds to ErbB2 with an association constant of at least 10¹² M⁻¹; (4) it binds to ErbB2 with an association constant of at least 10¹¹ M′¹; (5) it binds to ErbB2 with an association constant of at least 10¹⁰ M⁻¹; (6) it binds to ErbB2 with an association constant of at least 10⁶ M⁻¹; (7) it binds to ErbB2 with an association constant of at least 10⁶ M⁻¹; (8) it bind's to ErbB2 with a dissociation constant of 500 nM or less; (9) it binds to ErbB2 with a dissociation constant of 10 nM or less; (10) it binds to ErbB2 with a dissociation constant of 150 μM or less; (11) it binds to ErbB2 with a dissociation constant of 60 μM or less.

### III. Nucleic Acids, Cloning and Expression Systems

In another aspect, the invention provides isolated nucleic acids encoding an anti-ErbB2 binding protein of the invention. The nucleic acids may comprise DNA or RNA, and they may be synthetic (completely or partially) or recombinant (completely or partially). Reference to a nucleotide sequence as set out herein encompasses a DNA molecule with the specified sequence, and encompasses a RNA molecule with the specified sequence in which U is substituted for T.

The invention also contemplates nucleic acids that comprise a coding sequence for a CDR1, CDR2 or CDR3, a frame-work sequence (including FR1, FR2,

FR3 and/or FR4), a V_{H} domain, a V_{L} domain, or combinations thereof, as disclosed herein, or a sequence substantially identical thereto (e.g., a sequence at least 85%, 90%, 95%, 96%, 97%, 98%, 99% or higher identical thereto, or that is capable of hybridizing under stringent conditions to the sequences disclosed).

In one embodiment, the isolated nucleic acid has a nucleotide sequence encoding a heavy chain variable region and/or a light chain variable region of an anti-ErbB2 binding protein comprising at least one heavy chain CDR or light chain CDR, respectively, chosen from the CDR amino acid sequences found in SEQ ID Nos:1-62, or a sequence encoding a CDR that differs by one or two amino acids from the CDR sequences set forth herein. In some embodiments, the nucleic acid encodes an anti-ErbB2 binding protein comprising one, two, or all 3 heavy chain CDRs, one, two or all 3 light chain CDRs or all 6 CDRS in any of an specifically exemplified antibody.

The nucleic acid can encode only the light chain or the heavy chain variable region, or can also encode an antibody light or heavy chain constant region, operatively linked to the corresponding variable region. In one embodiment, the light chain variable region is linked to a constant region chosen from a kappa or a lambda constant region. The light chain constant region may also be a human kappa or lambda type. In another embodiment, the heavy chain variable region is linked to a heavy chain constant region of an antibody isotype chosen from IgG (e.g., IgG1, IgG₂, IgG₃, IgG₄), IgM, IgA₁, IgA₂, IgD, and IgE. The heavy chain constant region may be an IgG (e.g., an IgGᵢ) isotype.

The nucleic acid compositions of the present invention, while often in the native sequence (of cDNA or genomic DNA or mixtures thereof) except for modified restriction sites and the like, may be mutated in accordance with standard techniques to provide gene sequences. For coding sequences, these mutations, may affect amino acid sequence as desired. In particular, nucleotide sequences substantially identical to or derived from native V, D, J, constant, switches and other such sequences described herein are contemplated (where “derived” indicates that a sequence is identical or modified from another sequence).

In one embodiment, the nucleic acid differs (e.g., differs by substitution, insertion, or deletion) from that of the sequences provided (e.g., as follows: by at least one but less than 10, 20, 30, or 40 nucleotides; at least one but less than 1%, 5%, 10% or 20% of the nucleotides in the subject nucleic acid). Also within the invention are ErbB2 binding proteins encoded by a nucleic acid that hybridizes under stringent conditions to a nucleic acid specifically exemplified herein or to its complement. If necessary for this analysis the sequences should be aligned for maximum homology. “Looped out” sequences from deletions or insertions, or mismatches, are considered differences. The difference may be at a nucleotide(s) encoding a non-essential residue(s), or the difference may be a conservative substitution(s).

The invention also provides nucleic acid constructs in the form of plasmids, vectors, transcription or expression cassettes, that comprise at least one nucleic acid as described herein as well as a host cell that comprises at least one nucleic acid described herein. Suitable host cells for the expression of a binding protein of the invention well be well known in the art and include mammalian, plant, insects, bacterial or yeast cells.

Also provided are the methods of making an anti-ErbB2 antibody of the invention that is encoded by the nucleic acid(s) comprising sequence described herein. The method comprises culturing host cells under appropriate conditions to express the protein from the nucleic acid. Following expression and production, the encoded pp may be isolated and/or purified using any suitable technique, then used as appropriate. The method can also include the steps of fusing a nucleic acid encoding a scFv with nucleic acids encoding a Fc portion of an antibody and expressing the fused nucleic acid in a cell. The method can also include a step of germlining.

Antigen-binding fragments, V_{H} and/or V_{L} domains, and encoding nucleic acid molecules and vectors may be isolated and/or purified from their natural environment, in substantially pure or homogenous form, or, in the case of nucleic acid, free or substantially free of nucleic acid or genes of origin other than the sequence encoding a polypeptide with the require function.

Systems for cloning and expressing polypeptides in a variety of host cells are known in the art. Cells suitable for producing antibodies are described in, for example, Fernandez et al. (1999) Gene Expression Systems, Academic Press, eds. In brief, suitable host cells include mammalian cells, insect cells, plant cells, yeast cells, or prokaryotic cells, e.g., E. coli. Mammalian cells available in the art for heterologous polypeptide expression include lymphocytic cell lines (e.g., NSD), HEK293 cells, Chinese hamster ovary (CHO) cells, COS cells, HeLa cells, baby hamster kidney cells, oocyte cells, and cells from a transgenic animal, e.g., mammary epithelial cell.

In one embodiment, all or a portion of an anti-ErbB2 antibody selected from S1R2A_CS_{—}1F7, S1R2A_CS_{—}1D11, S1R2C_CS_{—}1D3, S1R2C_CS_{—}1H12, S1R2A_CS_{—}1D3, S1R3B2_BMV_{—}1E1, S1R3C1_CS_{—}1D3, S1R3B2_DP47_{—}1E8, S1R3B2_BMV_{—}1G2, S1R3B2_BMV_{—}1H5, S1R3C1_CS_{—}1A6, 51R3B2_DP47_{—}1C9, S1R3B2_DP47_{—}1E10, S1R3C1_CS_{—}1B10, S1R3A1_BMV_{—}1F3, S1R3B1_BMV_{—}1G11, S1R3A1_BMV_{—}1G4, S1R3B1_BMV_{—}1H11, S1R3A1_CS_{—}1B9, S1R3B1_BMV_{—}1H9, S1R3A1_CS_{—}11310, S1R3B1_BMV_{—}1C12, 51R3C1_BMV_{—}1H11 or S1R3B1_BMV_{—}1A1 is expressed in HEK293 or CHO cells. In other embodiments, one or more nucleic acids encoding an anti-ErbB2 binding protein of the invention are placed under the control of a tissue-specific promoter (e.g., a mammary specific promoter) and the antibodies are produced in transgenic animals. For example, the antibodies are secreted into the milk of the transgenic animal, such as a transgenic cow, pig, horse, sheep, goat or rodent.

Suitable vectors may be chosen or constructed to contain appropriate regulatory sequences, including promoter sequences, terminator sequences, polyadenylation sequences, enhancer sequences, marker genes, and other sequences. The vectors may also contain a plasmid or viral backbone. For details, see Sambrook et al., Molecular Cloning: A Laboratory Manual, 2nd ed., Cold Spring Harbor Laboratory Press (1989). Many established techniques used with vectors, including the manipulation, preparation, mutagenesis, sequencing, and transfection of DNA, are described in Current Protocols in Molecular Biology, Second Edition, Ausubel et al. eds., John Wiley & Sons (1992).

A nucleic acid encoding all or part of an anti-ErbB2 binding protein of the invention may be introduced into a host cell by any readily available means. For eukaryotic cells, suitable transfection techniques may include calcium phosphate, DEAE-Dextran, electroporation, liposome-mediated transfection, and transduction using retrovirus or other viruses, e.g., vaccinia or baculovirus. For bacterial cells, suitable techniques may include calcium chloride transformation, electroporation, and transfection using bacteriophage. DNA introduction may be followed by a selection method (e.g., drug resistance) to select cells that contain the nucleic acid.

### IV. Therapeutic Uses of Anti-ErbB2 Binding Proteins

Anti-ErbB2 binding proteins of the invention may be ErbB2 agonists or antagonists. An agonist ErbB2 binder of the invention increases HER2 tyrosine phosphorylation in the absence or presence of other HER2 agonists such as Heregulin or Epidermal Growth Factor (EGF). Certain HER2 agonists of the invention increase phosphorylation of HER2 pathway proteins. In some embodiments, the agonist of the invention increase phosphorylation of AKT, MAPK and/or ERK. In some embodiments, the HER2 agonist of the invention decreases proliferation and/or increases cell death of a cancer cell, in vitro and in vivo.

Anti-ErbB2 binding proteins that act as antagonists to ErbB2 can be used to reduce at least one ErbB2-mediated activity, such as reducing ErbB2-mediated tyrosine phosphorylation, decreased heterodimerization of ErbB2 with other ERBB-family members, decreased ErbB2-mediated cell signalling and decreased growth or proliferation of ErbB2-expressing cells. In one embodiment, anti-ErbB2 binding proteins of the invention are used in a method for decreasing tumor growth, the method comprising contacting an ErbB2 expressing cell with a binding protein of the invention to modulate cell proliferation, cytolytic activity, cytokine secretion, or chemokine secretion.

Accordingly, the binding proteins of the invention can be used to directly or indirectly inhibit or reduce the activity (e.g., proliferation, differentiation, and/or survival) of cells expressing ErbB2, and, thus, can be used to treat a variety of disorders including hyperproliferative disorders.

The binding proteins of the invention can be used to treat hyperproliferative disorders associated with activity of ErbB2 by administering the antibodies in an amount sufficient to inhibit or reduce hyperproliferation and/or to increase cell death, such as by apoptosis of ErbB2 expressing cells in a subject and allowing the antibodies to treat or prevent the disorder. ErbB2 is expressed in a number of cancers including, but not limited to, breast, bladder, cervical, ovarian, prostate, testicular, oral, colorectal, lung and pancreatic, cancers and in childhood medulloblastoma, oral squamous cell carcinoma, gastric cancer cholangio carcinoma, osteosarcoma, primary Fallopian tube carcinoma, salivary gland tumors and synovial sarcoma. Binding proteins of the invention may be used to inhibit the progression of neoplasms, e.g. squamous cell carcinomas, basal cell carcinomas, transitional cell papillomas and carcinomas, adenomas, adenocarcinoma. According to the invention, an anti-ErbB2 binding protein of the invention can be administered to a subject in need thereof as part of a regimen that comprises another therapeutic modality, such as surgery or radiation.

### V. Combination Therapy

According to the invention, a composition suitable for pharmaceutical use comprising at least one anti-ErbB2 binding protein further comprises at least one additional therapeutic agent. The therapy is useful for treating ErbB2-mediated pathological conditions or disorders including cancer. The term “in combination” in this context means that the binding protein composition and the additional therapeutic agent are given as part of a treatment regimen. In some embodiments, the anti-ErbB2 binding protein is administered substantially contemporaneously, either simultaneously or sequentially. In some embodiments, in which administration is sequential, at the onset of administration of the second agent, the first of the two agents is still detectable at effective concentrations at the site of treatment. In another embodiment, if given sequentially, at the onset of administration of the second compound, the first of the two compounds is not detectable at effective concentrations at the site of treatment.

For example, the combination therapy can include at least one anti-ErbB2 binding protein of the invention co-formulated with, co-administered with, or administered as part of the same therapeutic regimen as at least one additional therapeutic agent. The additional agents may include at least but is not limited to mitotic inhibitors, alkylating agents, anti-metabolites, intercalating antibiotics, growth factor inhibitors, cell cycle inhibitors, enzymes, topoisomerase inhibitors, biological response modifiers, antibodies, cytotoxics, antiproliferative agents, kinase inhibitors, angiogenesis inhibitors, growth factor inhibitors, cox-I inhibitors, cox-II inhibitors, radiation, cell cycle inhibitors, enzymes, anti-hormones, statins, and anti-androgens.

In other embodiments, at least one anti-ErbB2 binding protein can be co-formulated with, and/or co-administered with, at least one anti-inflammatory drug, immunosuppressant, metabolic inhibitor, and enzymatic inhibitor.

In other embodiments, an anti-ErbB2 antibody can be used in combination with at least one binding protein, such as an antibody, directed at other cancer targets. Another aspect of the present invention accordingly relates to kits for carrying out the administration of the anti-ErbB2 binding protein alone or in combination with other therapeutic agents. In one embodiment, the kit comprises at least one anti-ErbB2 binding protein formulated in a pharmaceutical carrier, and at least one additional therapeutic agent, formulated as appropriate in one or more separate pharmaceutical preparations.

In one embodiment, the present inventive binding proteins can be administered in combination with (e.g., prior to, concurrently with, or subsequent to) one or more other therapeutic agents. Such therapeutic agents include, for example, cytotoxic agents that inhibit or prevent the function of cells and/or causes destruction of cells. The term is intended to include radioactive isotopes (e.g. I131, I125, Y90 and Re186), chemotherapeutic agents, growth inhibitory agents, cytokine, and toxins such as enzymatically active toxins of bacterial, fungal, plant or animal origin, or fragments thereof.

Examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel (TAXOL®, Bristol-Myers Squibb Oncology, Princeton, N.J.) and docetaxel (TAXOTERE®, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylomithine (DMFO); retinoic acid; esperamicins; capecitabine; and pharmaceutically acceptable salts, acids or derivatives of any of the above. Also included are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

A growth inhibitory agent when used herein refers to a compound or composition that inhibits growth of a cell, especially an ErbB2-overexpressing cancer cell either in vitro or in vivo. In the context of the present invention, the growth inhibitory agent can be one that significantly reduces the percentage of ErbB2 overexpressing cells in S phase and the binding proteins of the present invention may potentially sensitize the cells to such an S phase agent. S-phase blockers include the vincas (vincristine and vinblastine), taxol, and topo II inhibitors such as doxorubicin, daunorubicin, etoposide, and bleomycin. Examples of growth inhibitory agents include agents that block cell cycle progression (at a place other than S phase), include agents that induce G1 arrest and M-phase arrest. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogens, and antineoplastic drugs” by Murakami et al. (W B Saunders: Philadelphia, 1995), especially p. 13.

Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor, fibroblast growth factor; prolactin; placental lactogen; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β; platelet-growth factor; transforming growth factors (TGFs) such as TGF-α and TGF-β; insulin-like growth factor-I and -II; erythropoietin (EPO); osteoinductive factors; interferons such as interferon-α, β, and -γ; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1α, IL-2, IL-3, IL-4, IL-5, IL-6, IL-7, IL-8, IL-9, IL-11, IL-12; a tumor necrosis factor such as TNF-α or TNF-β; and other polypeptide factors including LIF and kit ligand (KL). As used herein, the term cytokine includes proteins from natural sources or from recombinant cell culture and biologically active equivalents of the native sequence cytokines.

The invention also pertains to immunoconjugates comprising the binding proteins described herein conjugated to a cytotoxic agent such as a chemotherapeutic agent, toxin (e.g. an enzymatically active toxin of bacterial, fungal, plant or animal origin, or fragments thereof), or a radioactive isotope (i.e., a radioconjugate).

Chemotherapeutic agents useful in the generation of such immunoconjugates have been described above. Enzymatically active toxins and fragments thereof which can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes. A variety of radionuclides are available for the production of radioconjugated anti-ErbB2 binding proteins. Examples include 212Bi, 131I, 131In, 90 Y and 186Re.

Immunoconjugates comprising a member of the potent family of antibacterial and antitumor agents, known collectively as the calicheamicins or the LL-E33288 complex, (see U.S. Pat. No. 4,970,198 (1990)) are also contemplated. The most potent of the calicheamicins is designated γ 1, which is herein referenced simply as gamma. These compounds contain a methyltrisulfide that can be reacted with appropriate thiols to form disulfides, at the same time introducing a functional group such as a hydrazide or other functional group that is useful in attaching a calicheamicin derivative to a carrier. (See U.S. Pat. No. 5,053,394). Conjugation methods for preparing monomeric calicheamicin derivative/carrier have been disclosed (see U.S. Pat. No. 5,712,374 and U.S. Pat. No. 5,714,586, incorporated herein in their entirety).

Conjugates of the binding protein and cytotoxic agent can be made using a variety of bifunctional protein coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al. Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the binding protein.

Effective amounts of the other therapeutic agents are well known to those skilled in the art. However, it is well within the skilled artisan's purview to determine the other therapeutic agent's optimal effective amount range. The binding proteins of the present invention and the other therapeutic agent(s) can act additively or, alternatively, synergistically. In one embodiment of the invention, where another therapeutic agent(s) is administered to an animal, either the effective amount of the binding protein of the present invention or the other therapeutic agent(s) can be administered in an amount that is less than its effective amount would be where the other therapeutic agent is not administered. In this case, without being bound by theory, it is believed that the two (or more) act synergistically.

### VI. Diagnostic Uses

In a further aspect, a binding protein of the invention may also be used to detect the presence of ErbB2 or ErbB2 expressing cells in a biological sample. By correlating the presence or level of ErbB2 with a medical condition, one of skill in the art can diagnose the associated medical condition, including cancer.

Binding protein-based, including antibody-based detection methods are well known in the art, and include ELISA, radioimmunoassays, immunoblots, Western blots, flow cytometry, immunofluorescence, immunoprecipitation, and other related techniques. The antibodies may be provided in a diagnostic kit that incorporates at least one of these procedures to detect ErbB2. The kit may contain other components, packaging, instructions, or other material to aid the detection of the protein and use of the kit.

Binding proteins of the invention may be modified with detectable markers, including ligand groups (e.g., biotin), fluorophores and chromophores, radioisotopes, electron-dense reagents, or enzymes. Enzymes are detected by their activity. For example, horseradish peroxidase is detected by its ability to convert tetramethylbenzidine (TMB) to a blue pigment, quantifiable with a spectrophotometer. Other suitable binding partners include biotin and avidin, IgG and protein A, and other receptor-ligand pairs known in the art.

Binding proteins of the invention can also be functionally linked (e.g., by chemical coupling, genetic fusion, non-covalent association or otherwise) to at least one other molecular entity, such as another antibody (e.g., a bispecific or a multispecific antibody), toxins, radioisotopes, cytotoxic or cytostatic agents, among others for therapeutic use. Other permutations and possibilities are apparent to those of ordinary skill in the art, and they are considered equivalents within the scope of this invention.

Further, the anti-ERRB2 binding proteins can be used to detect the presence, isolate, and/or to quantitate ErbB2-expressing cells in a sample from a subject or by in vivo imaging.

### VII. Pharmaceutical Compositions and Methods of Administration

In still another aspect, the invention provides compositions comprising an anti-ErbB2 binding protein of the invention. The compositions may be suitable for pharmaceutical use and administration to patients. The compositions comprise a binding protein of the present invention and a pharmaceutically acceptable carrier. The composition may optionally comprise a pharmaceutical excipient. As used herein, “pharmaceutical excipient” includes solvents, dispersion media, coatings, antibacterial and antifungal agents, isotonic and absorption delaying agents, etc., that are compatible with pharmaceutical administration. Use of these agents for pharmaceutically active substances is well known in the art. The compositions may also contain other active compounds providing supplemental, additional, or enhanced therapeutic functions. The pharmaceutical compositions may also be included in a container, pack, or dispenser together with instructions for administration.

A pharmaceutical composition of the invention is formulated to be compatible with its intended route of administration. Methods to accomplish the administration are known to those of ordinary skill in the art. Pharmaceutical compositions may be topically or orally administered, or capable of transmission across mucous membranes. Examples of administration of a pharmaceutical composition include oral ingestion or inhalation. Administration may also be intravenous, intraperitoneal, intramuscular, intracavity, subcutaneous, cutaneous, or transdermal.

Solutions or suspensions used for intradermal or subcutaneous application typically include at least one of the following components: a sterile diluent such as water, saline solution, fixed oils, polyethylene glycol, glycerine, propylene glycol, or other synthetic solvent; antibacterial agents such as benzyl alcohol or methyl parabens; antioxidants such as ascorbic acid or sodium bisulfite; chelating agents such as ethylenediaminetetraacetic acid (EDTA); buffers such as acetate, citrate, or phosphate; and tonicity agents such as sodium chloride or dextrose. The pH can be adjusted with acids or bases. Such preparations may be enclosed in ampoules, disposable syringes, or multiple dose vials.

Solutions or suspensions used for intravenous administration include a carrier such as physiological saline, bacteriostatic water, Cremophor EL™ (BASF, Parsippany, N.J.), ethanol, or polyol. In all cases, the composition must be sterile and fluid for easy syringability. Proper fluidity can often be obtained using lecithin or surfactants. The composition must also be stable under the conditions of manufacture and storage. Prevention of microorganisms can be achieved with antibacterial and antifungal agents, e.g., parabens, chlorobutanol, phenol, ascorbic acid, thimerosal, etc. In many cases, isotonic agents (sugar), polyalcohols (mannitol and sorbitol), or sodium chloride may be included in the composition. Prolonged absorption of the composition can be accomplished by adding an agent that delays absorption, e.g., aluminum monostearate and gelatin.

Oral compositions include an inert diluent or edible carrier. The composition can be enclosed in gelatin or compressed into tablets. For the purpose of oral administration, the antibodies can be incorporated with excipients and placed in tablets, troches, or capsules. Pharmaceutically compatible binding agents or adjuvant materials can be included in the composition. The tablets, troches, and capsules, may contain (1) a binder such as microcrystalline cellulose, gum tragacanth or gelatin; (2) an excipient such as starch or lactose, (3) a disintegrating agent such as alginic acid, Primogel, or corn starch; (4) a lubricant such as magnesium stearate; (5) a glidant such as colloidal silicon dioxide; or (6) a sweetening agent or a flavoring agent.

The composition may also be administered by a transmucosal or transdermal route. For example, antibodies that comprise a Fc portion may be capable of crossing mucous membranes in the intestine, mouth, or lungs (via Fc receptors). Transmucosal administration can be accomplished through the use of lozenges, nasal sprays, inhalers, or suppositories. Transdermal administration can also be accomplished through the use of a composition containing ointments, salves, gels, or creams known in the art. For transmucosal or transdermal administration, penetrants appropriate to the barrier to be permeated are used. For administration by inhalation, the antibodies are delivered in an aerosol spray from a pressured container or dispenser, that contains a propellant (e.g., liquid or gas) or a nebulizer.

In certain embodiments, the binding proteins of this invention are prepared with carriers to protect against rapid elimination from the body. Biodegradable polymers (e.g., ethylene vinyl acetate, polyanhydrides, polyglycolic acid, collagen, polyorthoesters, polylactic acid) are often used. Methods for the preparation of such formulations are known by those skilled in the art. Liposomal suspensions can be used as pharmaceutically acceptable carriers too. The liposomes can be prepared according to established methods known in the art (U.S. Pat. No. 4,522,811).

The binding proteins or compositions of the invention are administered in therapeutically effective amounts as described. Therapeutically effective amounts may vary with the subject's age, condition, sex, and severity of medical condition. Appropriate dosage may be determined by a physician based on clinical indications. The binding proteins or compositions may be given as a bolus dose to maximize the circulating levels of protein for the greatest length of time. Continuous infusion may also be used after the bolus dose.

As used herein, the term “subject” is intended to include human and non-human animals. Subjects may include a human patient having a disorder characterized by cells that express ErbB2, e.g., a cancer cell or an immune cell. The term “non-human animals” of the invention includes all vertebrates, such as non-human primates, sheep, dogs, cows, chickens, amphibians, reptiles, etc.

Examples of dosage ranges that can be administered to a subject can be chosen from: 1 μg/kg to 20 mg/kg, 1 μg/kg to 10 mg/kg, 1 μg/kg to 1 mg/kg, 10 μg/kg to 1 mg/kg, 10 μg/kg to 100 μg/kg, 100 μg/kg to 1 mg/kg, 250 μg/kg to 2 mg/kg, 250 μg/kg to 1 mg/kg, 500 μg/kg to 2 mg/kg, 500 μg/kg to 1 mg/kg, 1 mg/kg to 2 mg/kg, 1 mg/kg to 5 mg/kg, 5 mg/kg to 10 mg/kg, 10 mg/kg to 20 mg/kg, 15 mg/kg to 20 mg/kg, 10 mg/kg to 25 mg/kg, 15 mg/kg to 25 mg/kg, 20 mg/kg to 25 mg/kg, and 20 mg/kg to 30 mg/kg (or higher). These dosages may be administered daily, weekly, biweekly, monthly, or less frequently, for example, biannually, depending on dosage, method of administration, disorder or symptom(s) to be treated, and individual subject characteristics. Dosages can also be administered via continuous infusion (such as through a pump). The administered dose may also depend on the route of administration. For example, subcutaneous administration may require a higher dosage than intravenous administration.

In certain circumstances it may be advantageous to formulate compositions in dosage unit form for ease of administration and uniformity of dosage. Dosage unit form as used herein refers to physically discrete units suited for the patient. Each dosage unit contains a predetermined quantity of antibody calculated to produce a therapeutic effect in association with the carrier. The dosage unit depends on the characteristics of the antibodies and the particular therapeutic effect to be achieved.

Toxicity and therapeutic efficacy of the composition can be determined by standard pharmaceutical procedures in cell cultures or experimental animals, e.g., determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Binding proteins that exhibit large therapeutic indices may be less toxic and/or more therapeutically effective.

The data obtained from the cell culture assays and animal studies can be used to formulate a dosage range in humans. The dosage of these compounds may lie within the range of circulating antibody concentrations in the blood, that includes an ED₅₀ with little or no toxicity. The dosage may vary within this range depending upon the dosage composition form employed and the route of administration. For any antibody used in the present invention, the therapeutically effective dose can be estimated initially using cell culture assays. A dose may be formulated in animal models to achieve a circulating plasma concentration range that includes the IC₅₀ (i.e., the concentration of antibody that achieves a half-maximal inhibition of symptoms). The effects of any particular dosage can be monitored by a suitable bioassay. Examples of suitable bioassays include DNA replication assays, transcription-based assays and ErbB2 binding assays.

### EXAMPLES

### Example 1

### Selection of Anti-ErbB2 scFv's

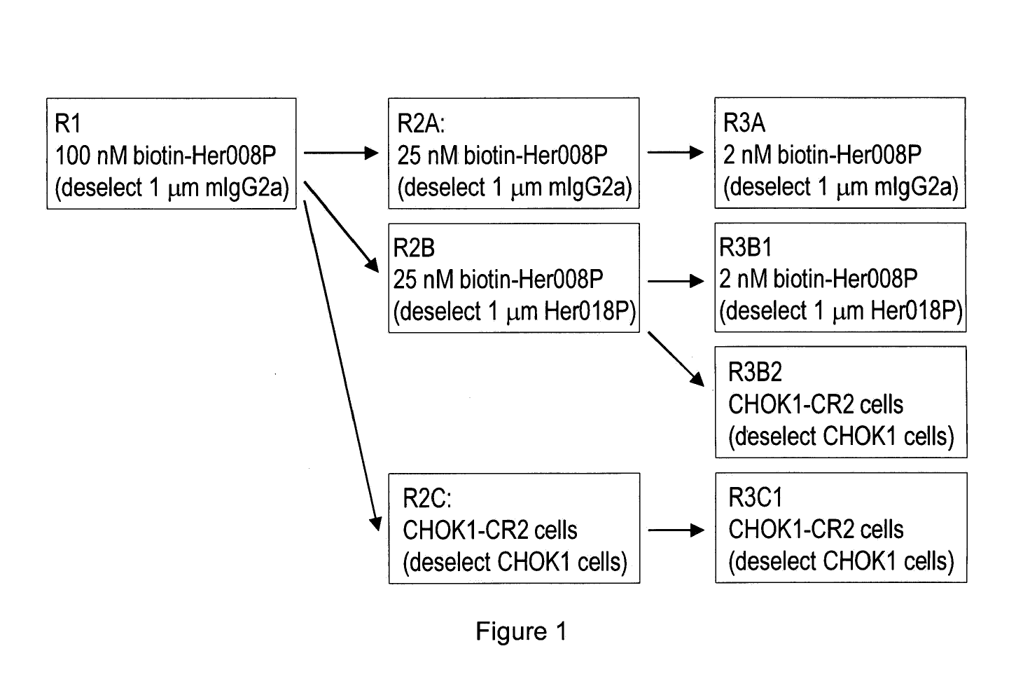

Single chain fragment variable (scFv) moieties that bind to the extracellular domain (ECD) of Her2 (ErbB2) were identified following three rounds of selection using three phagemid libraries: the Bone Marrow Vaughan (BMV) library (Vaughan et al, 1996), the combined spleen (CS) library and the DP47 library (unpublished). Several Her2-Fc proteins or cell lines expressing various forms of Her2 were used during the selection and subsequent screening steps (see Table 3). The selection strategies are outlined in FIG. 1.

Selection Using Biotinylated HER2 Proteins

For selections involving biotinylated protein, aliquots of phage and magnetic streptavidin beads (Dynabeads M-280 streptavidin) were blocked separately in 3% milk/PBS for 1 hour at room temperature in a rotary mixer (20 rpm). Each selection was preceded by a de-selection step. For de-selection, blocked phage were incubated with the pre-blocked magnetic beads and incubated for one hour on a rotary shaker (20 rpm). The de-selected library was collected by pelleting the beads using a magnetic separator. A 1 μM concentration of a non-biotinylated competitor protein (eg, irrelevant MlgG2a protein) was added to the de-selected phage and incubated for a further hour.

Biotinylated selection antigen (at various concentrations as indicated in FIG. 1) was incubated with the de-selected phage library for 2 hours at room temp on a rotary mixer (20 rpm) followed by a 15 minute incubation with pre-blocked magnetic beads. Beads were separated using a magnetic separator and washed 10 times with PBS/0.1% Tween 20 and 3 times with PBS. Bound phage were eluted by incubation with a 10 ug/ml solution of trypsin in PBS for 30 minutes at 37° C. (100 rpm) followed by separation from the magnetic beads.

Selection Using Cells Expressing HER2ECD or ECD Fragments

For selections involving cells, approximately 4×10⁷ de-selection cells (ie. cells not expressing the antigen of interest) and 2×10⁷ capture (i.e., selection) cells (cells expressing the antigen of interest) were collected using PBS/5 mM EDTA and washed twice with PBS. Cells were blocked with 3% milk/1% BSA/PBS for 1 hour at 4° C. on a rotary mixer (20 rpm). De-selection cells were collected by centrifugation, re-suspended in blocked phage and incubated at 4° C. as before. Both the capture and de-selection cells were pelleted and the capture cells were resuspended with the de-selected phage supernatant and incubated at 4° C. as before. The capture cells were washed three times with cold PBS/0.1% Tween 20 and three times with cold PBS. Phage were eluted by re-suspending the cells in a 10 μg/ml trypsin solution and incubated for 30 min at 37° C. (100 rpm). Eluted phage were harvested in the supernatant following centrifugation of cells. Eluted phage were used to infect 10 ml of an E. coli TG1 culture that had been grown to mid-logarithmic phase (corresponding to an OD₆₀₀ of ˜0.5). Bacteria were infected with phage for 1 hour at 37° C. with shaking at 150 rpm, concentrated following a centrifugation step and plated on 2×TY agar bioassay plates containing 2% glucose and 100 ug/ml ampicillin (2×TYAG). Various dilutions of E. coli culture infected with either input or output phage were also plated on 2×TYAG agar to determine phage titers. Following overnight growth at 30° C., 10 ml of 2×TYAG medium was added to each bioassay plate and the cells were re-suspended by scraping the bacterial lawn. Glycerol was added to this cell suspension to give a final concentration of 17% and stored in aliquots at −80° C. until further use. To rescue phage for the next round of selection, 100 μl of this cell suspension was used to inoculate 20 ml 2×TYAG medium, that was grown at 37° C. (300 rpm) to an OD₆₀₀ of 0.3-0.5. Cells were then super-infected with 3.3 μl of MK13K07 helper phage and incubated at 37° C. (150 rpm) for 1 hour. The cells were then centrifuged and the pellet re-suspended in a kanamycin/non-glucose containing medium (2×TY with 50 μg/ml kanamycin and 100 ug/ml ampicillin). This culture was grown overnight at 30° C. (300 rpm). Phage were harvested in the supernatant following centrifugation and were ready to use in the second and third rounds of selection as described in FIG. 1.

TABLE 3

| | | Sequence for Her2 region |
|---|---|---|
| Name | Description | of fusion protein |
| Her008P | Full-length extracellular | MELAALCRWGLLLALLPPGAASTQV |
| (Synonyms: | domain (ECD) of Her2 | CTGTDMKLRLPASPETHLDMLRHLY |
| ECD; SIIS; | expressed with a mlgG2a | QGCQVVQGNLELTYLPTNASLSFLQ |
| HER008) | Fc tail | DIQEVQGYVLIAHNQVRQVPLQRLR |
| | | IVRGTQLFEDNYALAVLDNGDPLNN |
| | | TTPVTGASPGGLRELQLRSLTEILK |
| | | GGVLIQRNPQLCYQDTILWKDIFHK |
| | | NNQLALTLIDTNRSRACHPCSPMCK |
| | | GSRCWGESSEDCQSLTRTVCAGG |
| | | CARCKGPLPTDCCHEQCAAGCTGP |
| | | KHSDCLACLHFNHSGICELHCPALV |
| | | TYNTDTFESMPNPEGRYTFGASCV |
| | | TACPYNYLSTDVGSCTLVCPLHNQE |
| | | VTAEDGTQRCEKCSKPCARVCYGL |
| | | GMEHLREVRAVTSANIQEFAGCKKI |
| | | FGSLAFLPESFDGDPASNTAPLQPE |
| | | QLQVFETLEEITGYLYISAWPDSLPD |
| | | LSVFQNLQVIRGRILHNGAYSLTLQ |
| | | GLGISWLGLRSLRELGSGLALIHHN |
| | | THLCFVHTVPWDQLFRNPHQALLH |
| | | TANRPEDECVGEGLACHQLCARGH |
| | | CWGPGPTQCVNCSQFLRGQECVE |
| | | ECRVLQGLPREYVNARHCLPCHPE |
| | | CQPQNGSVTCFGPEADQCVACAH |
| | | YKDPPFCVARCPSGVKPDLSYMPI |
| | | WKFPDEEGACQPCPINCTHSCVDL |
| | | DDKGCPAEQRASPLTSIIS |
| | | (SEQ ID NO: 242) |
| Her017P | Her2 ECD with a deletion | MELAALCRWGLLLALLPPGAASTQV |
| (Synonyms: | in the membrane proximal | CTGTDMKLRLPASPETHLDMLRHLY |
| EQR; | 9 amino acids expressed | QGCQVVQGNLELTYLPTNASLSFLQ |
| HER017) | with a mlgG2a Fc tail | DIQEVQGYVLIAHNQVRQVPLQRLR |
| | | IVRGTQLFEDNYALAVLDNGDPLNN |
| | | TTPVTGASPGGLRELQLRSLTEILK |
| | | GGVLIQRNPQLCYQDTILWKDIFHK |
| | | NNQLALTLIDTNRSRACHPCSPMCK |
| | | GSRCWGESSEDCQSLTRTVCAGG |
| | | CARCKGPLPTDCCHEQCAAGCTGP |
| | | KHSDCLACLHFNHSGICELHCPALV |
| | | TYNTDTFESMPNPEGRYTFGASCV |
| | | TACPYNYLSTDVGSCTLVCPLHNQE |
| | | VTAEDGTQRCEKCSKPCARVCYGL |
| | | GMEHLREVRAVTSANIQEFAGCKKI |
| | | FGSLAFLPESFDGDPASNTAPLQPE |
| | | QLQVFETLEEITGYLYISAWPDSLPD |
| | | LSVFQNLQVIRGRILHNGAYSLTLQ |
| | | GLGISWLGLRSLRELGSGLALIHHN |
| | | THLCFVHTVPWDQLFRNPHQALLH |
| | | TANRPEDECVGEGLACHQLCARGH |
| | | CWGPGPTQCVNCSQFLRGQECVE |
| | | ECRVLQGLPREYVNARHCLPCHPE |
| | | CQPQNGSVTCFGPEADQCVACAH |
| | | YKDPPFCVARCPSGVKPDLSYMPI |
| | | WKFPDEEGACQPCPINCTHSCVDL |
| | | DDKGCPAEQR |
| | | (SEQ ID NO: 243) |
| Her018P | Her2 ECD with a deletion | MELAALCRWGLLLALLPPGAASTQV |
| (Synonyms: | in the CR2 (Domain IV) | CTGTDMKLRLPASPETHLDMLRHLY |
| 1.8, | region expressed with a | QGCQVVQGNLELTYLPTNASLSFLQ |
| HER018) | mlgG2a Fc tail | DIQEVQGYVLIAHNQVRQVPLQRLR |
| | | IVRGTQLFEDNYALAVLDNGDPLNN |
| | | TTPVTGASPGGLRELQLRSLTEILK |
| | | GGVLIQRNPQLCYQDTILWKDIFHK |
| | | NNQLALTLIDTNRSRACHPCSPMCK |
| | | GSRCWGESSEDCQSLTRTVCAGG |
| | | CARCKGPLPTDCCHEQCAAGCTGP |
| | | KHSDCLACLHFNHSGICELHCPALV |
| | | TYNTDTFESMPNPEGRYTFGASCV |
| | | TACPYNYLSTDVGSCTLVCPLHNQE |
| | | VTAEDGTQRCEKCSKPCARVCYGL |
| | | GMEHLREVRAVTSANIQEFAGCKKI |
| | | FGSLAFLPESFDGDPASNTAPLQPE |
| | | QLQVFETLEEITGYLYISAWPDSLPD |
| | | LSVFQNLQVIRGRILHNGAYSLTLQ |
| | | GLGISWLGLRSLRELGSGLALIHHN |
| | | THLCFVHTVPWDQLFRNPHQALLH |
| | | TANRPEDECVGEGLACHQLCARGH |
| | | CWGPGPTQCVNCSQFLRGQECVE |
| | | ECRVLQGLPREYVNARHCLPCHPE |
| | | CQPQNGSVTCFGPEADQCVACAH |
| | | YKDPPFCVAR |
| | | (SEQ ID NO: 244) |
| Her054P | Domains I (L1) and II | MELAALCRWGLLLALLPPGAASTQV |
| (Synonyms: | (CR-1) of Her2 | CTGTDMKLRLPASPETHLDMLRHLY |
| L1-CR1; | expressed with a mlgG2a | QGCQVVQGNLELTYLPTNASLSFLQ |
| 1.0) | Fc tail | DIQEVQGYVLIAHNQVRQVPLQRLR |
| | | IVRGTQLFEDNYALAVLDNGDPLNN |
| | | TTPVTGASPGGLRELQLRSLTEILK |
| | | GGVLIQRNPQLCYQDTILWKDIFHK |
| | | NNQLALTLIDTNRSRACHPCSPMCK |
| | | GSRCWGESSEDCQSLTRTVCAGG |
| | | CARCKGPLPTDCCHEQCAAGCTGP |
| | | KHSDCLACLHFNHSGICELHCPALV |
| | | TYNTDTFESMPNPEGRYTFGASCV |
| | | TACPYNYLSTDVGSCTLVCPLHNQE |
| | | VTAEDGTQRCEKCSKPC |
| | | (SEQ ID NO: 245) |
| Full length | | MELAALCRWGLLLALLPPGAASTQV |
| HER2 | | CTGTDMKLRLPASPETHLDMLRHLY |
| | | QGCQVVQGNLELTYLPTNASLSFLQ |
| | | DIQEVQGYVLIAHNQVRQVPLQRLR |
| | | IVRGTQLFEDNYALAVLDNGDPLNN |
| | | TTPVTGASPGGLRELQLRSLTEILK |
| | | GGVLIQRNPQLCYQDTILWKDIFHK |
| | | NNQLALTLIDTNRSRACHPCSPMCK |
| | | GSRCWGESSEDCQSLTRTVCAGG |
| | | CARCKGPLPTDCCHEQCAAGCTGP |
| | | KHSDCLACLHFNHSGICELHCPALV |
| | | TYNTDTFESMPNPEGRYTFGASCV |
| | | TACPYNYLSTDVGSCTLVCPLHNQE |
| | | VTAEDGTQRCEKCSKPCARVCYGL |
| | | GMEHLREVRAVTSANIQEFAGCKKI |
| | | FGSLAFLPESFDGDPASNTAPLQPE |
| | | QLQVFETLEEITGYLYISAWPDSLPD |
| | | LSVFQNLQVIRGRILHNGAYSLTLQ |
| | | GLGISWLGLRSLRELGSGLALIHHN |
| | | THLCFVHTVPWDQLFRNPHQALLH |
| | | TANRPEDECVGEGLACHQLCARGH |
| | | CWGPGPTQCVNCSQFLRGQECVE |
| | | ECRVLQGLPREYVNARHCLPCHPE |
| | | CQPQNGSVTCFGPEADQCVACAH |
| | | YKDPPFCVARCPSGVKPDLSYMPI |
| | | WKFPDEEGACQPCPINCTHSCVDL |
| | | DDKGCPAEQRASPLTSIISAVVGILL |
| | | VVVLGVVFGILIKRRQQKIRKYTMRR |
| | | LLQETELVEPLTPSGAMPNQAQMRI |
| | | LKETELRKVKVLGSGAFGTVYKGIW |
| | | IPDGENVKIPVAIKVLRENTSPKANK |
| | | EILDEAYVMAGVGSPYVSRLLGICLT |
| | | STVQLVTQLMPYGCLLDHVRENRG |
| | | RLGSQDLLNWCMQIAKGMSYLEDV |
| | | RLVHRDLAARNVLVKSPNHVKITDF |
| | | GLARLLDIDETEYHADGGKVPIKWM |
| | | ALESILRRRFTHQSDVWSYGVTVW |
| | | ELMTFGAKPYDGIPAREIPDLLEKGE |
| | | RLPQPPICTIDVYMIMVKCWMIDSE |
| | | CRPRFRELVSEFSRMARDPQRFVVI |
| | | QNEDLGPASPLDSTFYRSLLEDDD |
| | | MGDLVDAEEYLVPQQGFFCPDPAP |
| | | GAGGMVHHRHRSSSTRSGGGDLT |
| | | LGLEPSEEEAPRSPLAPSEGAGSDV |
| | | FDGDLGMGAAKGLQSLPTHDPSPL |
| | | QRYSEDPTVPLPSETDGYVAPLTCS |
| | | PQPEYVNQPDVRPQPPSPREGPLP |
| | | AARPAGATLERPKTLSPGKNGVVK |
| | | DVFAFGGAVENPEYLTPQGGAAPQ |
| | | PHPPPAFSPAFDNLYYWDQDPPER |
| | | GAPPSTFKGTPTAENPEYLGLDVPV |
| | | (SEQ ID NO: 246) |

### Example 2

### Preparation of Phage or Crude Periplasmic Material for Use in ELISAs

ScFvs can be expressed either on the surface of a phage particle or in solution in the bacterial periplasmic space, depending upon the growth conditions used. To induce release of scFv into the periplasm, 96-deepwell plates containing 2×TY media with 0.1% glucose/100 μg/ml ampicillin were inoculated from thawed glycerol stocks (one clone per well) using the QPix2 Colony picker (Genetix) and grown at 37° C. (999 rpm) for ˜4 hours. Cultures were induced with IPTG at a final concentration of 0.02 mM and grown overnight at 30° C. (999 rpm). The contents of the bacterial periplasm (peripreps) were released by osmotic shock. Briefly, plates were centrifuged and pellets were resuspended in 150 μl HEPES periplasmic buffer (50 mM HEPES, pH7.4/0.5 mM EDTA/20% Sucrose), followed by the addition of 150 μl 1:5 HEPES:water and incubated on ice for 30 minutes. Plates were centrifuged and the scFv-containing supernatant was harvested.

To prepare phage expressing scFv on their surface, 96-well plates containing 150 μl 2×TY media with 2% glucose/100 μg/ml ampicillin were inoculated from thawed glycerol stocks as described above and grown at 37° C. (700 rpm) for ˜4 hours. 20 μl of a 1:1000 dilution of helper phage (˜2×10⁸ pfu) was added and the plates incubated for a further hour at 37° C. (300 rpm). Plates were centrifuged and the media was replaced with a kanamycin/non-glucose containing media (2×TY with 50 μg/ml kanamycin and 100 ug/ml ampicillin). Plates were grown overnight at 30° C. (700 rpm) and phage were harvested in the supernatant following centrifugation.

Thirty-one Her2-binding ScFv's were identified by three rounds of screenings as illustrated in FIG. 1. These ScFv's specifically bind to the ECD region of Her2.

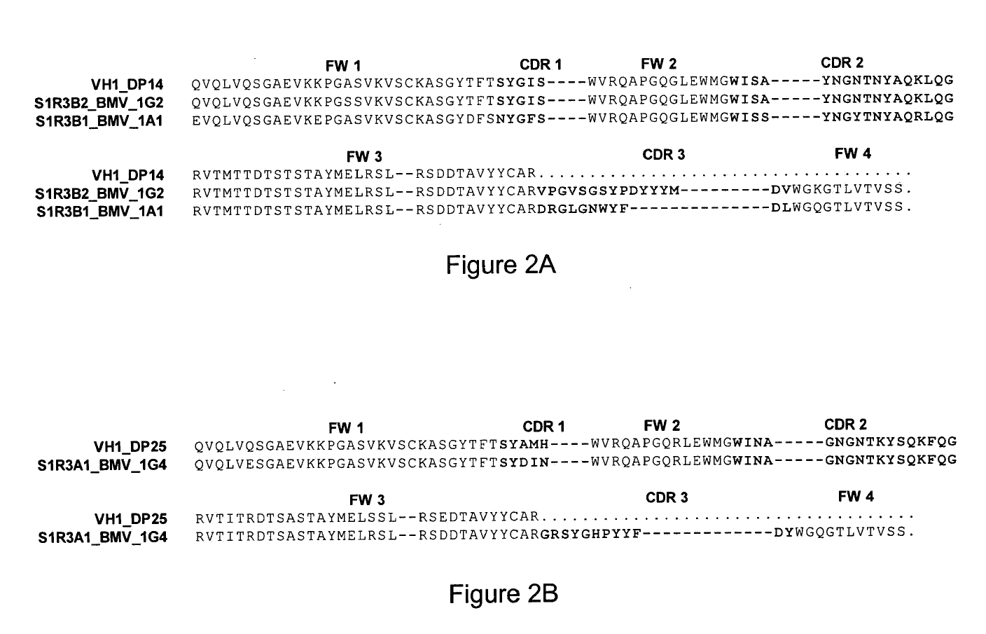

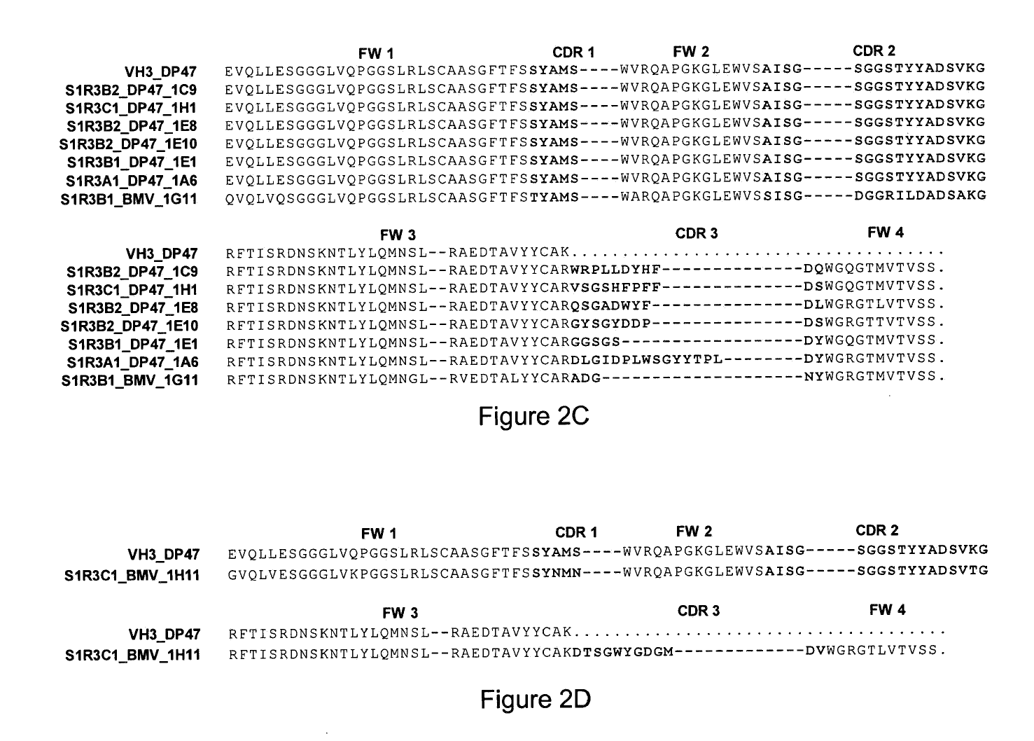

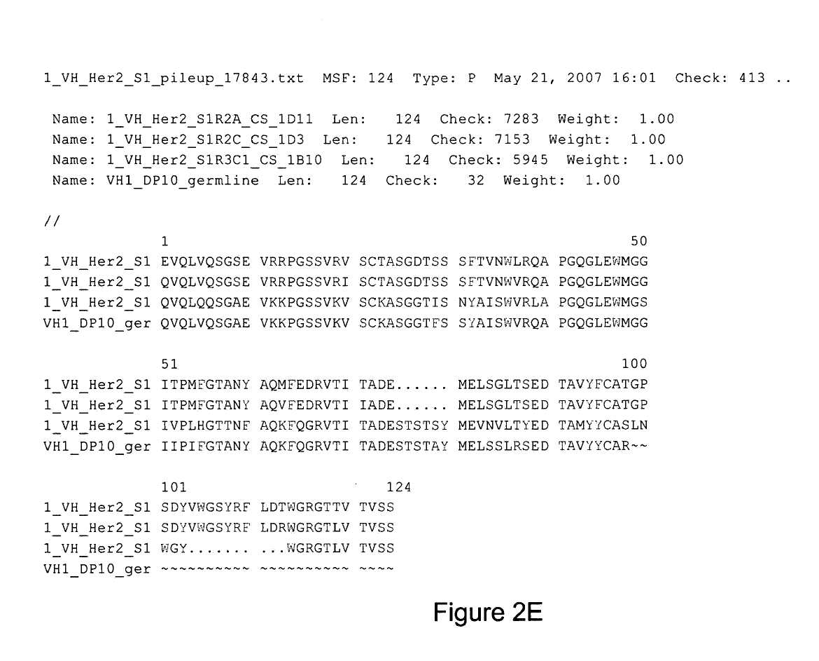

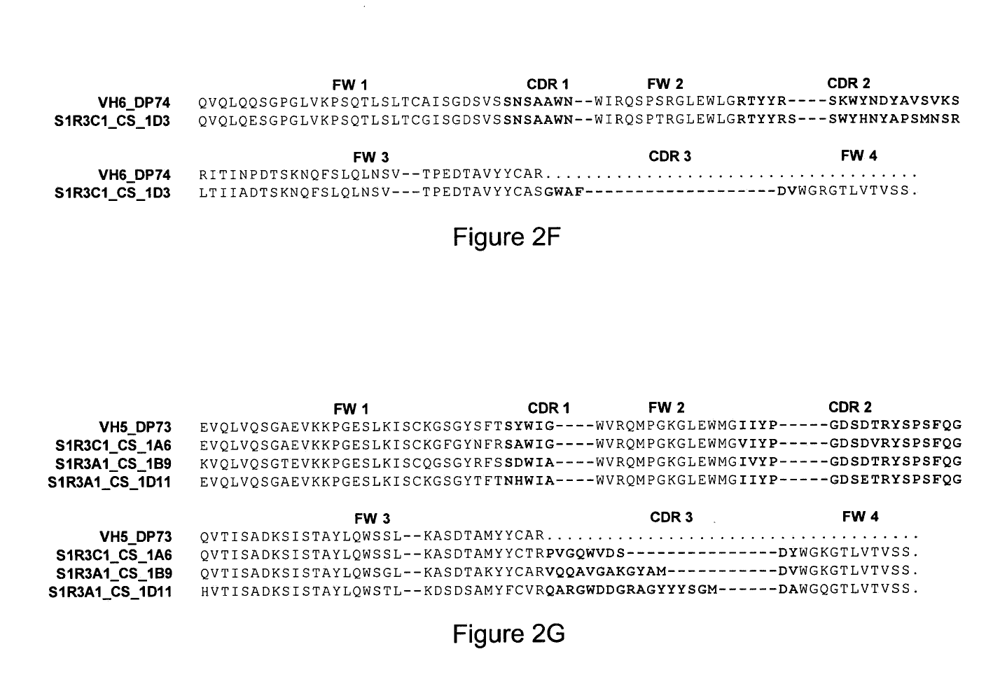

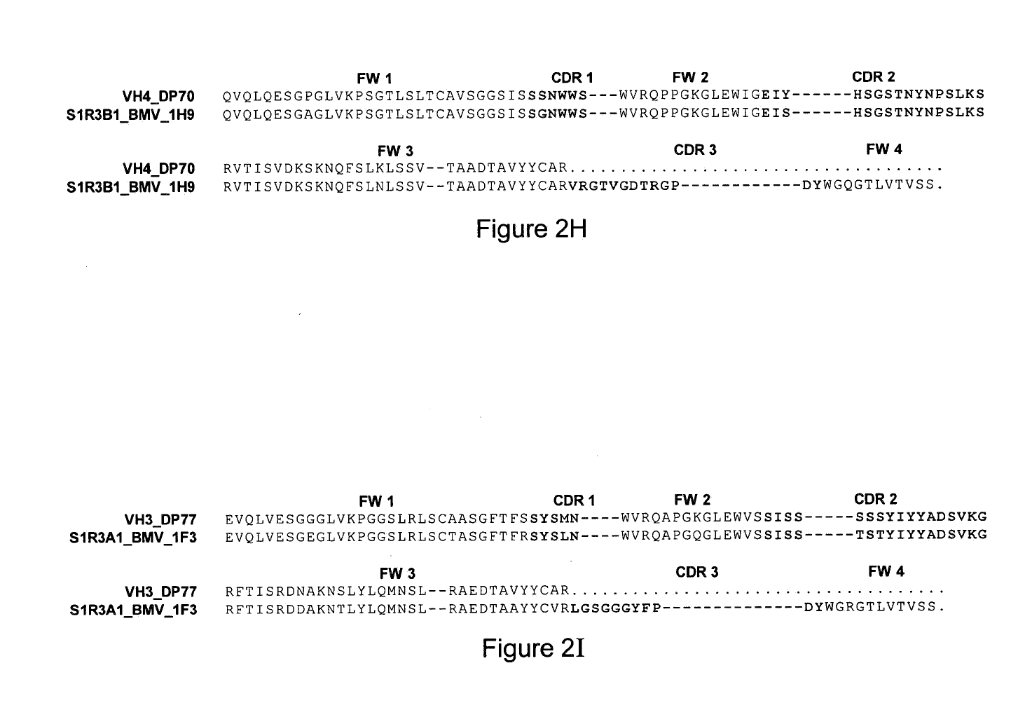

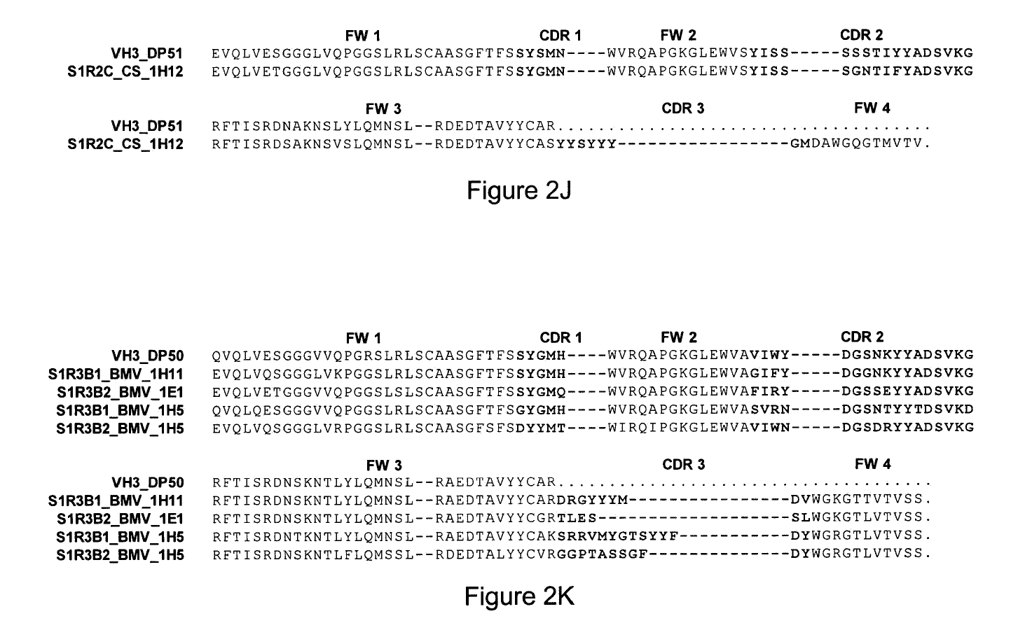

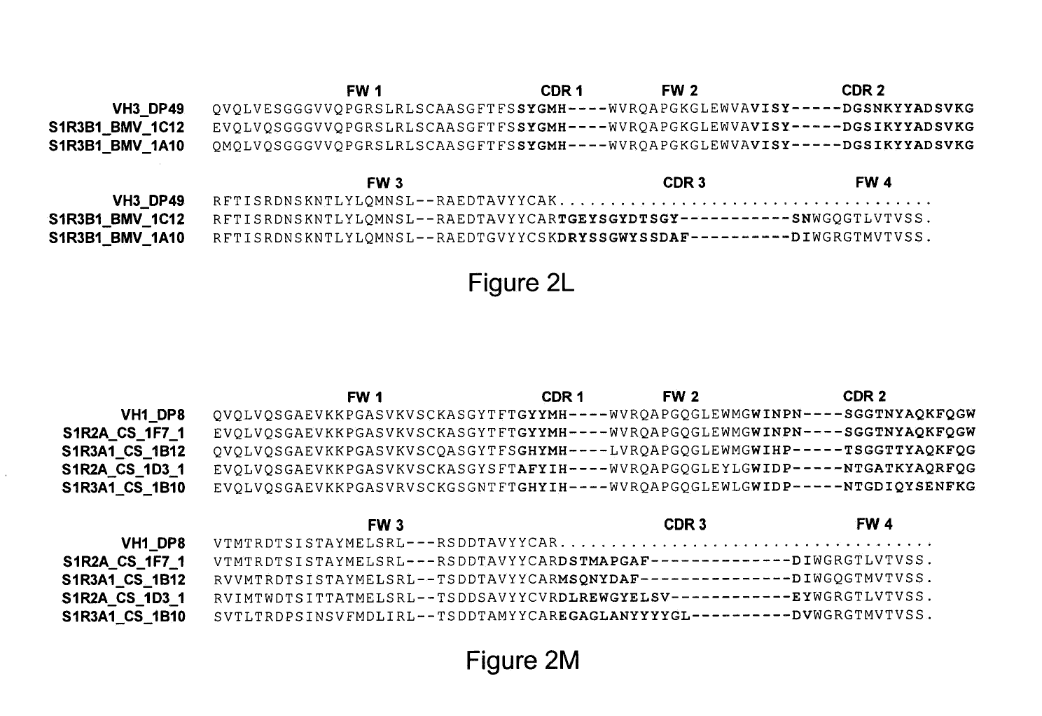

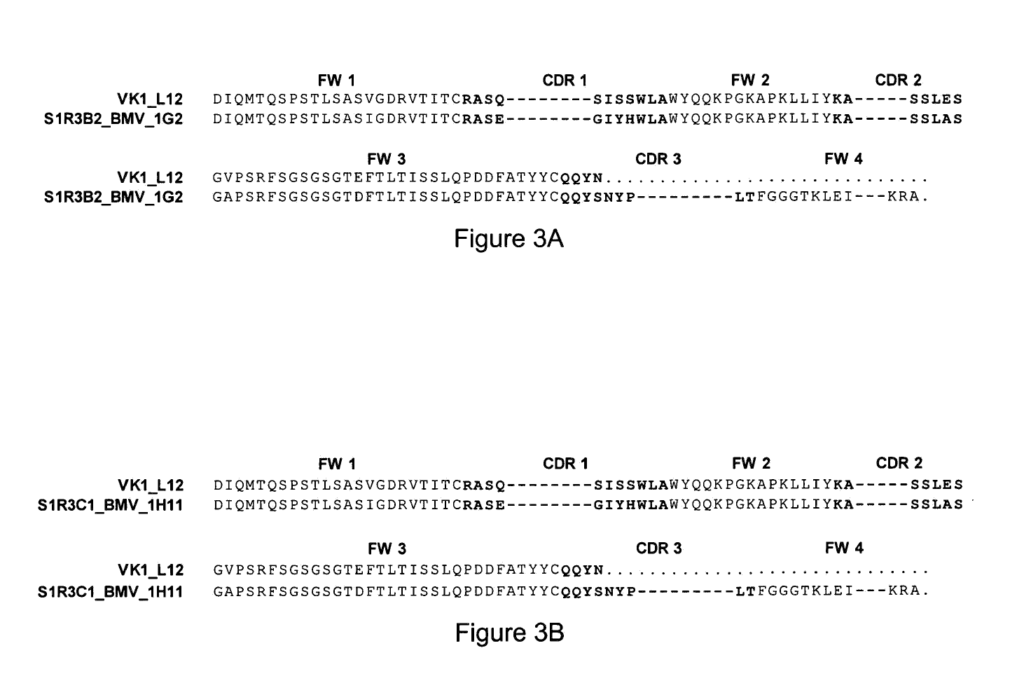

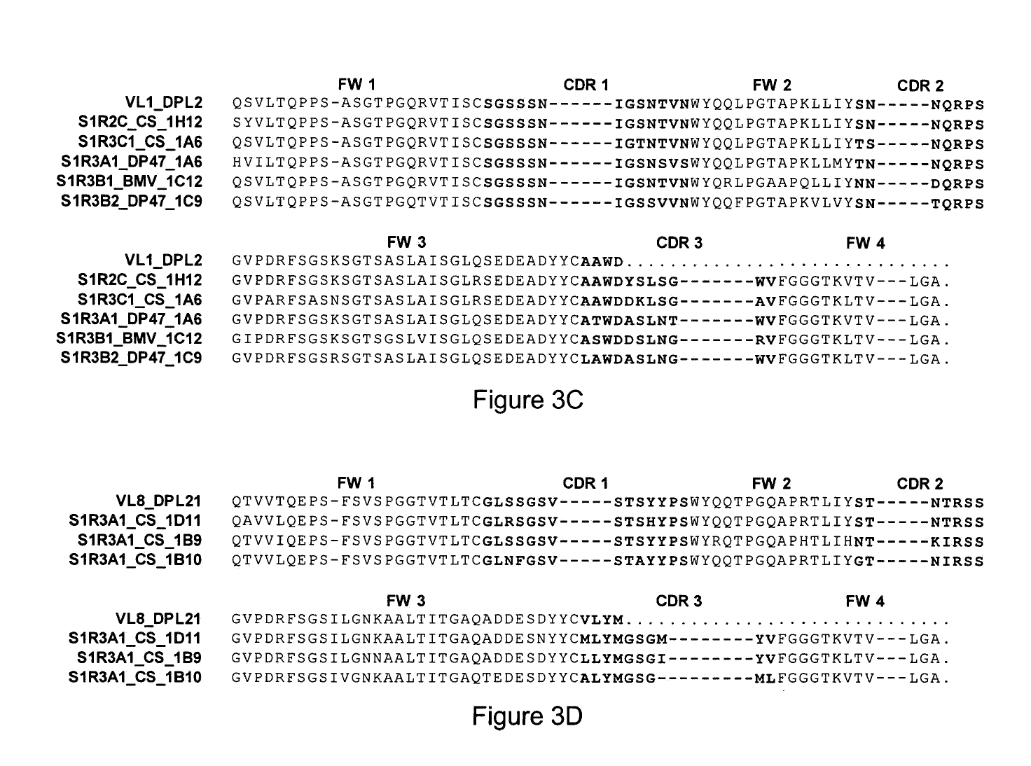

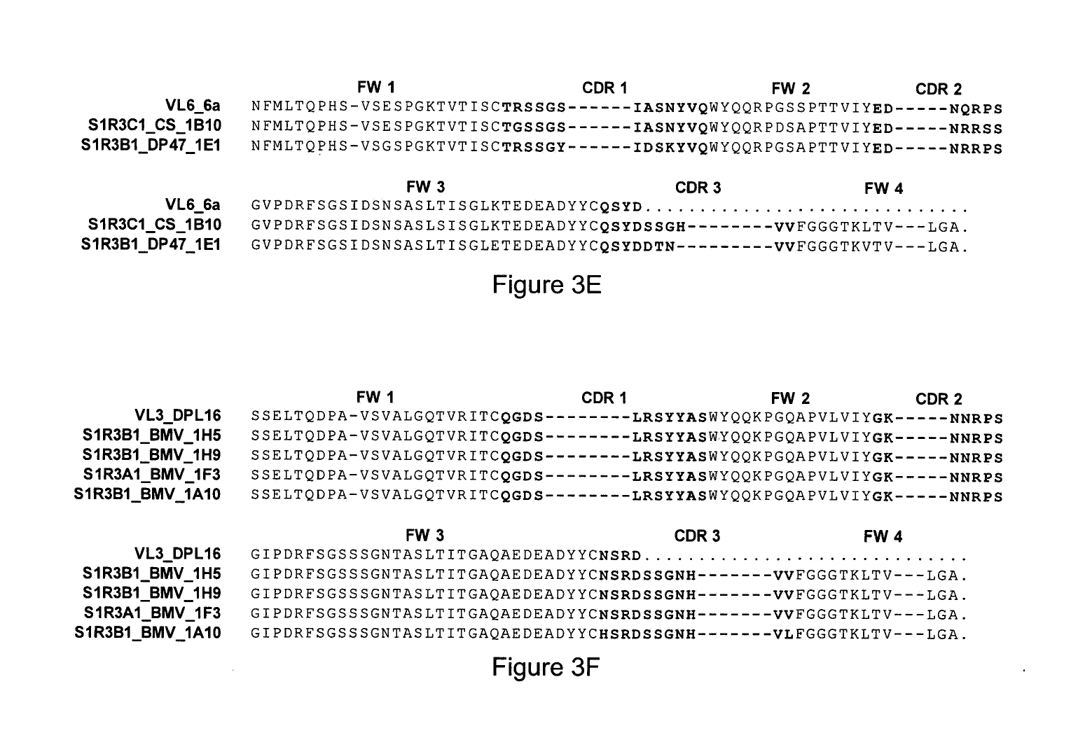

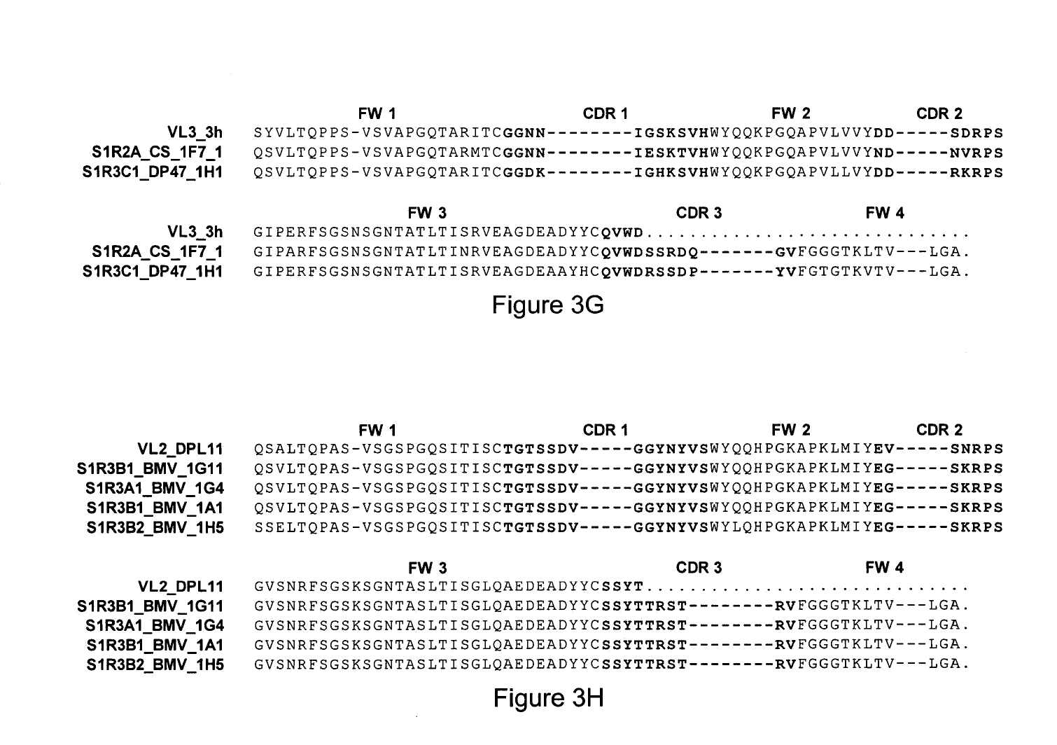

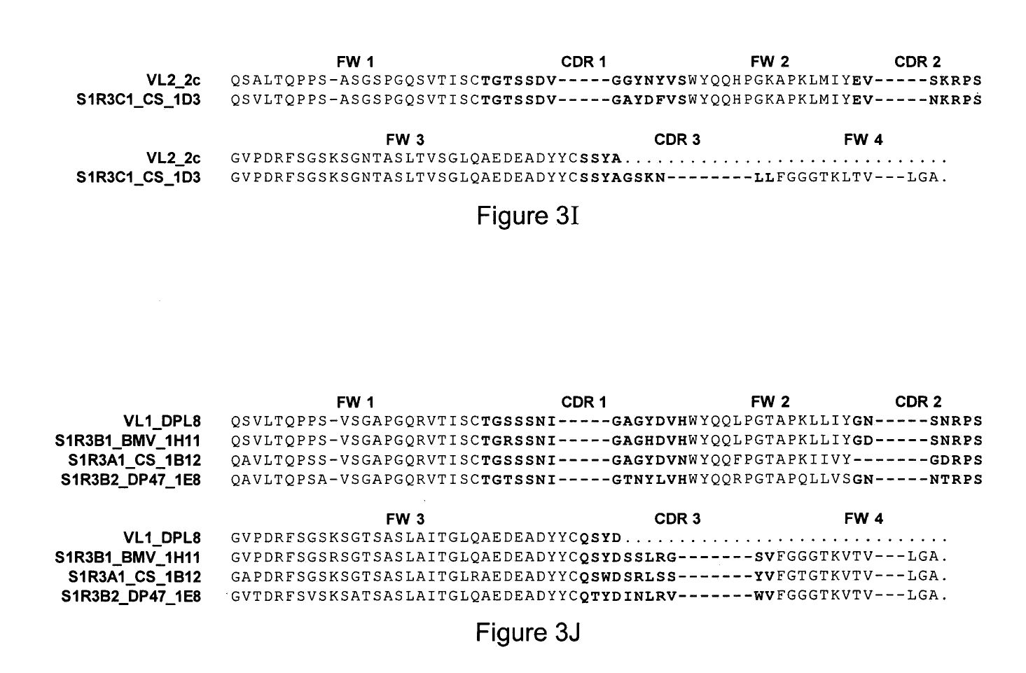

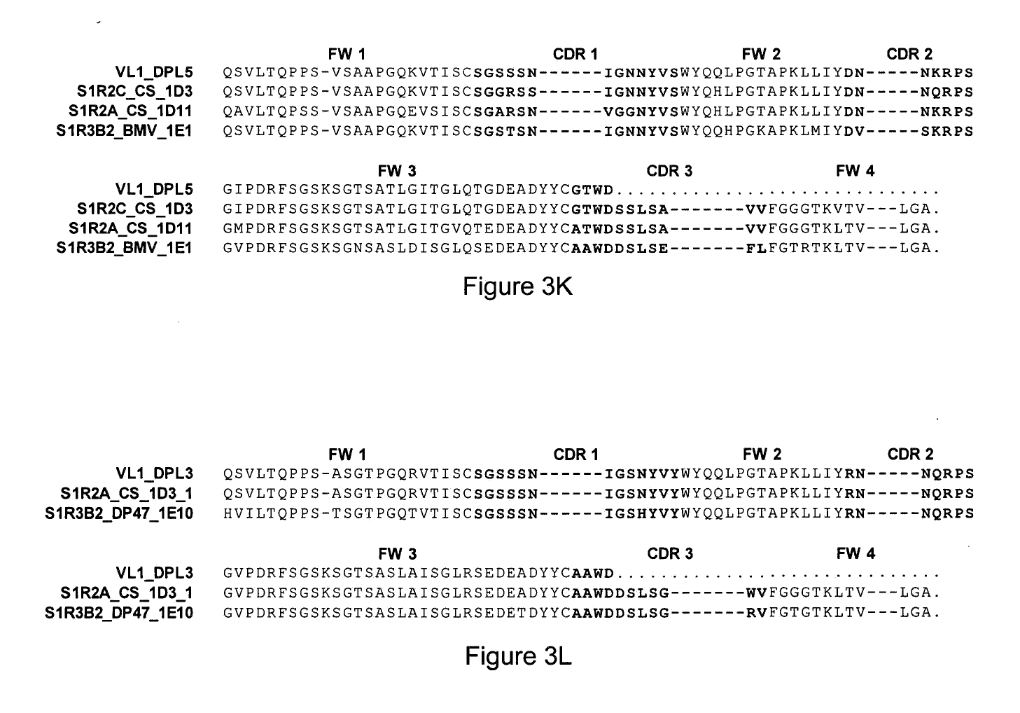

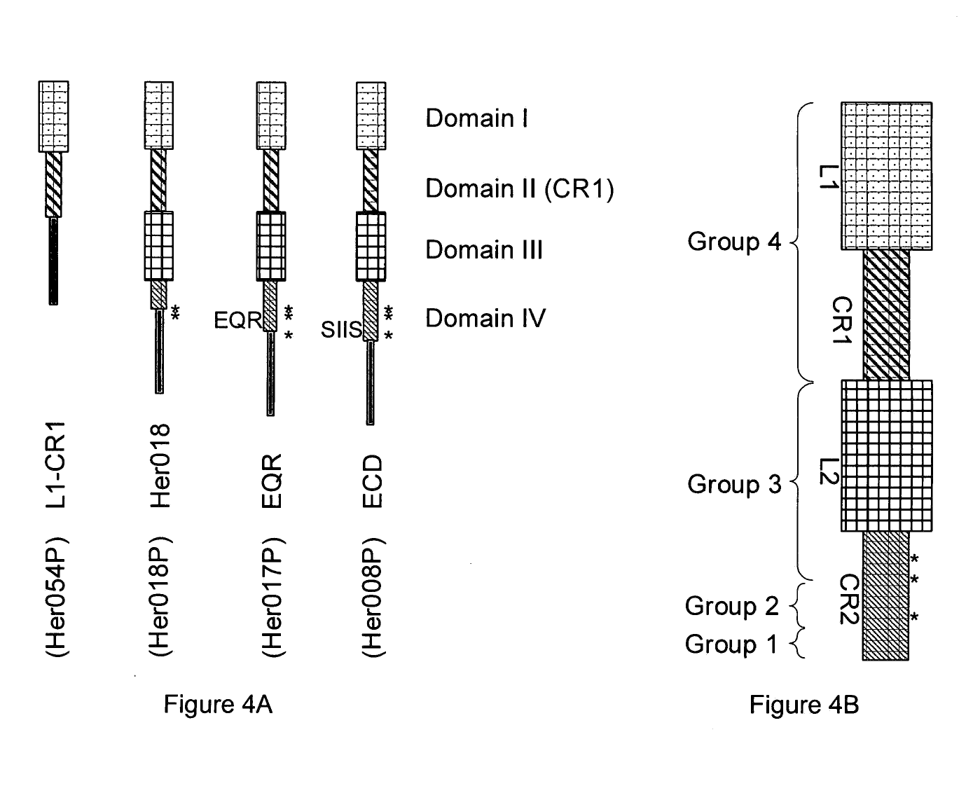

Among these thirty-one Her2-binding ScFv's, fourteen ScFv's were expressed on the surface of a phage particle for the purpose of screening. These ScFv's are: S1R2A_CS_{—}1F7, S1R2A_CS_{—}1D11, S1R2C_CS_{—}1D3, S1R2C_CS_{—}1H12, S1R2A_CS_{—}1D3, S1R3B2_BMV_{—}1E1, S1R3C1_CS_{—}1D3, S1R3B2_DP47_{—}1E8, S1R3B2_BMV_{—}1G2, S1R3B2_BMV_{—}1H5, S1R3C1_CS_{—}1A6, S1R3B2_DP47_{—}1C9, S1R3B2_DP47_{—}1E10, and S1R3C1_CS_{—}1B10 (FIGS. 2 and 3).

The remaining seventeen ScFv's were expressed in bacterial periplasm in soluble form for the purpose of screening: S1R3A1_BMV_{—}1F3, S1R3B1_BMV_{—}1G11, S1R3A1_BMV_{—}1G4, S1R3B1_BMV_{—}1H11, S1R3A1_CS_{—}1B9, S1R3B1_BMV_{—}1H9, S1R3A1_CS_{—}1B10, S1R3B1_BMV_{—}1C12, S1R3C1_BMV_{—}1H11, S1R3B1_BMV_{—}1A10, S1R3A1_CS_{—}1D11, S1R3C1_DP47_{—}1H1, S1R3A1_CS_{—}1B12, S1R3B1_BMV_{—}1H5, S1R3A1_DP47_{—}1A6, S1R3B1_DP47_{—}1E1, and S1R3B1_BMV_{—}1A1 (FIGS. 2 and 3).

### Example 3

### Elisa to Test Her2 Protein Construct Binding by scFvs Expressed in the E. coli Periplasm, on the Surface of Phage, or in Mammalian Cells as Fc Fusions

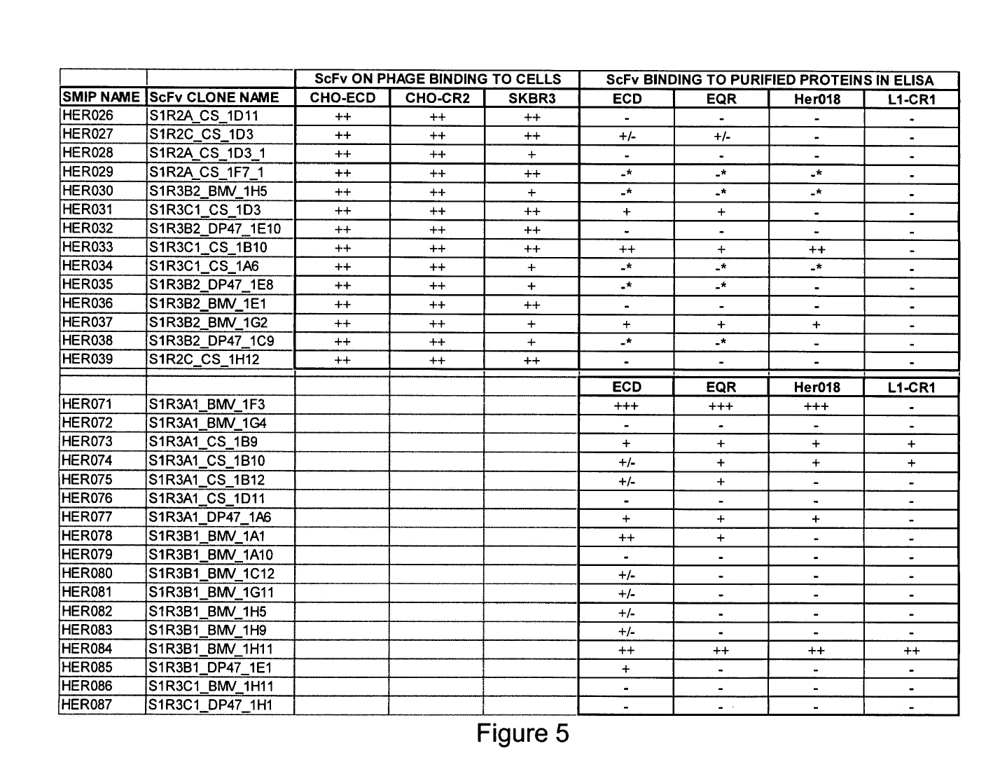

Various Her2-Fc proteins (e.g., Her008P, Her017P, Her018P, etc.) or a negative control murine IgG2a protein were coated overnight at 4° C. on 96-well Nunc Maxisorp at a concentration of 1 ug/ml in PBS. Alternatively, pre-blocked streptavidin-coated plates (Greiner) were coated with biotinylated Her2-Fc proteins for 1 hour at room temperature at a concentration of 1 ug/ml in block buffer (3% skim milk/1% BSA/PBS). Plates were washed three times using PBS and blocked for 1 hour at room temperature in 3% skim milk/1% BSA/PBS. Phage or peripreps were prepared as described above and were blocked for 1 hour at room temperature in an equal volume of 6% skim milk/1% BSA/PBS. Blocked plates were washed five times with PBS and 50 μl/well of blocked phage or periprep were transferred to the appropriate plates and incubated for 1 hour at room temperature. A 1 ug/ml solution of HERCEPTIN® (trastuzumab) (in blocking buffer) was added to well H12 of each plate to serve as a positive control. Plates were washed five times with PBS prior to the addition of a 1:250 dilution of anti-myc peroxidase (Roche), a 1:2500 dilution of anti-M13 peroxidase (Amersham Biosciences) or a 1:5000 or 1:1000 dilution of goat anti-human peroxidase (Southern Biotech) secondary antibody to detect bound scFv, phage, HERCEPTIN® (trastuzumab) or SMIP, respectively. Plates were incubated for a further hour at room temperature and washed seven times with PBS. Signal was developed using TMB, the reaction stopped with H₂SO₄ and the absorbance read at 450 nm on an Envision plate reader (Perkin Elmer). The results of these binding assays are shown in FIG. 5.

Alternatively, plates were coated with 1 ug/ml of a SMIP (Her030, Her033/Her067, Her018) or antibody (Herceptin®, positive control). SMIPs were used to capture 3-fold serial dilution (9-0 μg/ml) of soluble protein sample as follows: dimeric HER2 (HERB017), monomeric HER2 (HER155), or monomeric HER2 (shed ectodomain from SKBR3 supernatant). Captured soluble protein was detected using 0.1 mg/ml anti-c-Erb B2/c-Neu (Ab-5) mouse mAb (TA-1; binds ECD; Calbiochem) and detected using HRP-conjugated Goat anti-mouse IgG (Fcg Subclass 1 specific; Jackson ImmunoResearch).

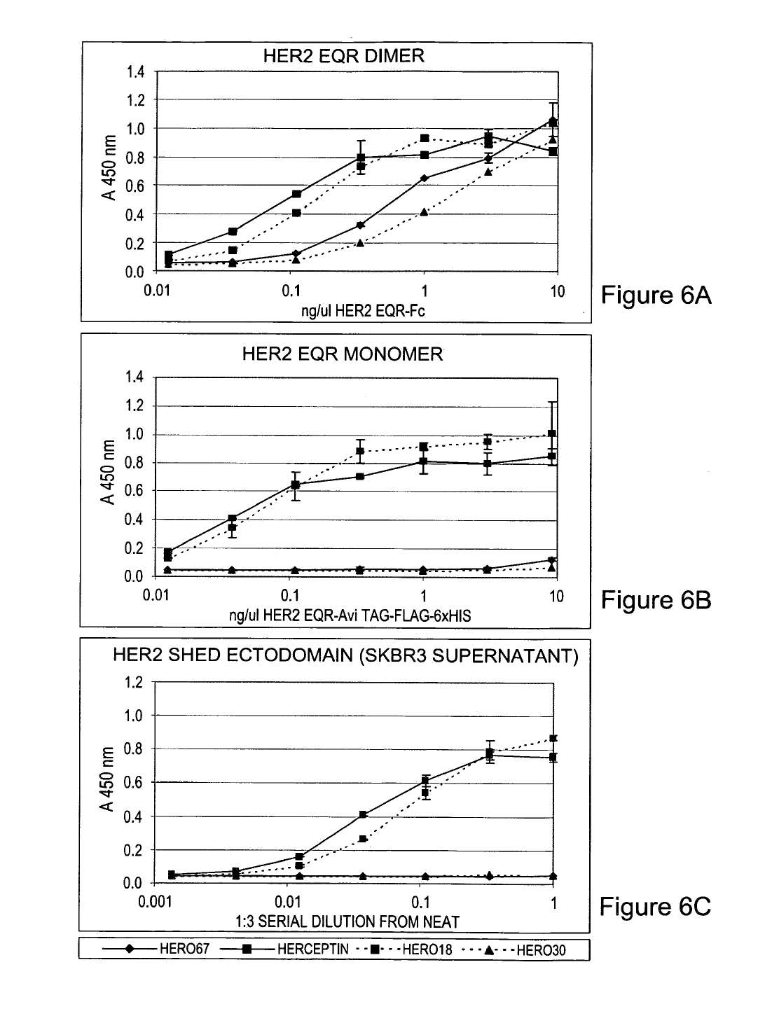

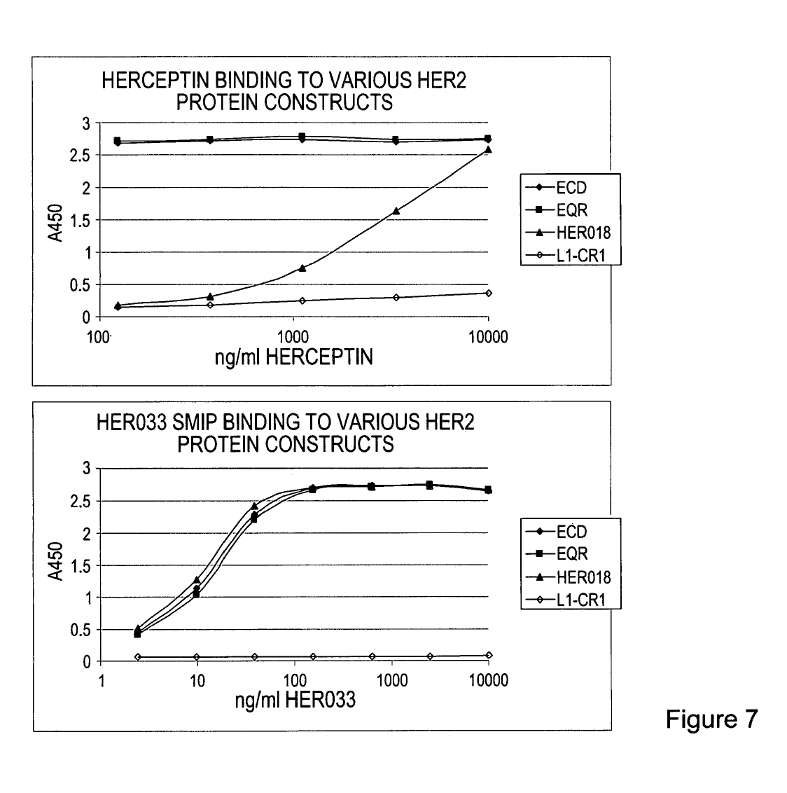

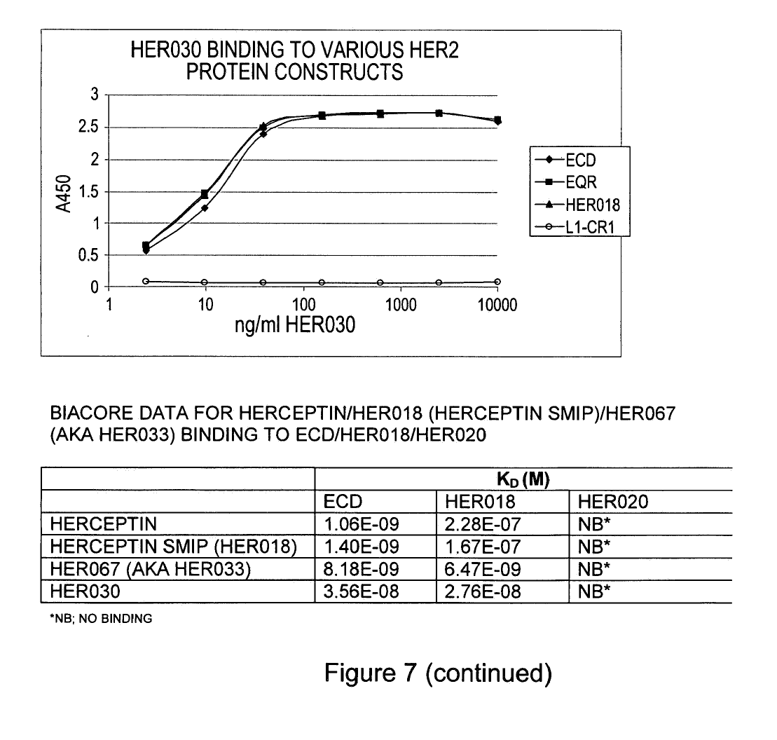

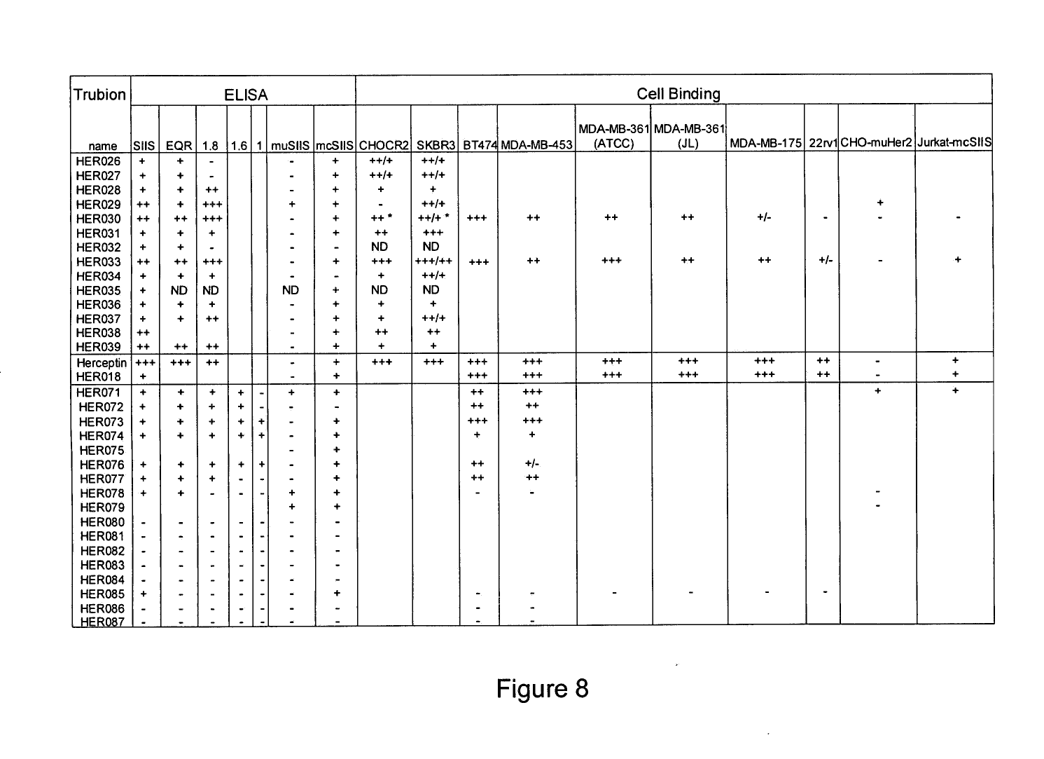

The results of the SMIP binding assays are shown in FIG. 6A-C, FIG. 7A-7D and FIG. 8. In FIG. 8, the binding of HER018, HER026-HER039 and Herceptin® (trastuzumab) to Her2 protein constructs was scored as −, +, ++ or +++, while the binding of HER071-HER087 to Her2 protein constructs was scored as a − or +.

### Example 4

### ELISA to Measure Binding of scFvs (Expressed in the Periplasm or on the Surface of Phage) to Her2-Expressed Cells

2×10⁴ CHOK1 cells/well were seeded in a 96-well tissue culture plate on Day 1 and incubated at 37° C./5% CO₂ for 2-4 days until a confluent monolayer was observed. Cells were washed five times with PBS (+Ca/Mg ions) and blocked for 1 hour at room temperature with 3% skim milk/1% BSA/PBS (+Ca/Mg ions). Phage or peripreps were prepared as described above and were blocked for 1 hour at room temperature in an equal volume of 6% skim milk/1% BSA/PBS (+Ca/Mg ions). Blocked plates were washed five times with PBS (+Ca/Mg ions) and 50 μl/well of blocked phage or periprep were transferred to the appropriate plates and incubated for 1 hour at room temperature. A 1 ug/ml solution of HERCEPTIN® (trastuzumab) (in blocking buffer) was added to well H12 of each plate to serve as a positive control. Plates were washed five times with PBS (+Ca/Mg ions) prior to the addition of a 1:250 dilution of anti-myc peroxidase (Roche), a 1:2500 dilution of anti-M13 peroxidase (Amersham Biosciences) or a 1:5000 dilution of goat anti-human (Southern Biotech) secondary antibody to detect bound scFv, phage or HERCEPTIN® (trastuzumab) respectively. Plates were incubated for a further hour at room temperature and washed ten times with PBS (+Ca/Mg ions). Signal was developed using TMB, the reaction stopped with H₂SO₄ and the absorbance read at 450 nm on an ENVISION plate reader (Perkin Elmer). The results of these binding assays are shown in FIG. 5.

Alternatively, the cell lines tested for SMIP binding included SKBR3, BT474, 22rv1, MDA-MB-175, MDA-MB-453, MDA-MB-361 (ATCC), MDA-MB-361 (JL), and Ramos (Her2⁻/CD20⁺ control). The SMIPs tested included Her067 (c.f. Her033), Her094 (c.f. Her030), and Her018, while the controls used included Herceptin® (trastuzumab), Rituxan® (anti-CD20 mAb rituximab), and CD20-SMIP.

Each well of a 6 well plate was seeded with 2×10⁵ cells and incubated overnight at 37° C./5% CO₂. Cells were then treated with antibody or SMIP (at 10 ug/ml final) (in triplicate) and incubated for another 24 or 48 hours. After incubation, the cells were pulsed with 50 uM BrdU (Sigma) for 30 minutes at 37° C., the media was removed, and the cells were treated with trypsin (except Ramos) and then 3−3.5×10⁵ cells per well were stained in 100 μl Staining Buffer in the presence or absence of a SMIP or antibody one of three different concentrations (ranging from 200 nM to 0.27 nM). The SMIP or antibody treatment was removed and the cells were washed three times with PBS, pH 7.2-7.4 with 0.1% TWEEN®-20 (PBS-T). A secondary antibody (5 ug/ml Alexa Fluor 488-conjugated Goat anti-Human IgG; Molecular Probes) was then added and incubated for 1-2 hours at room temperature. The secondary antibody was removed and the cells washed again three times with PBS-T. The cells were then fixed in 1% paraformaldehyde in Staining Buffer and analyzed 1 hour to 1 day later.

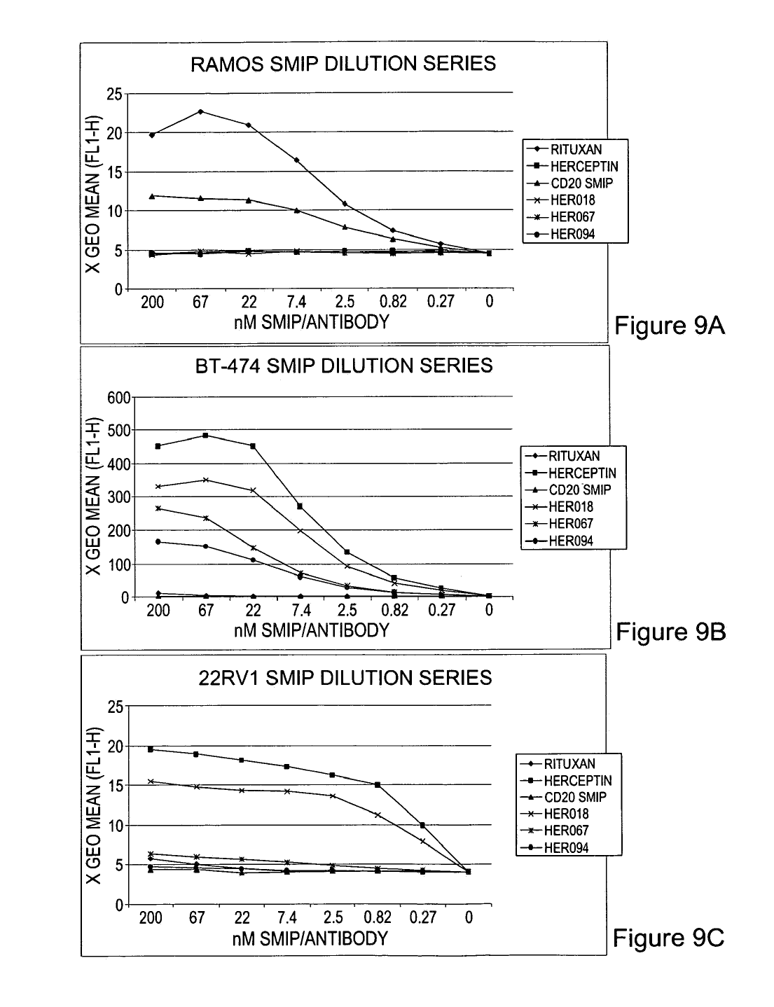

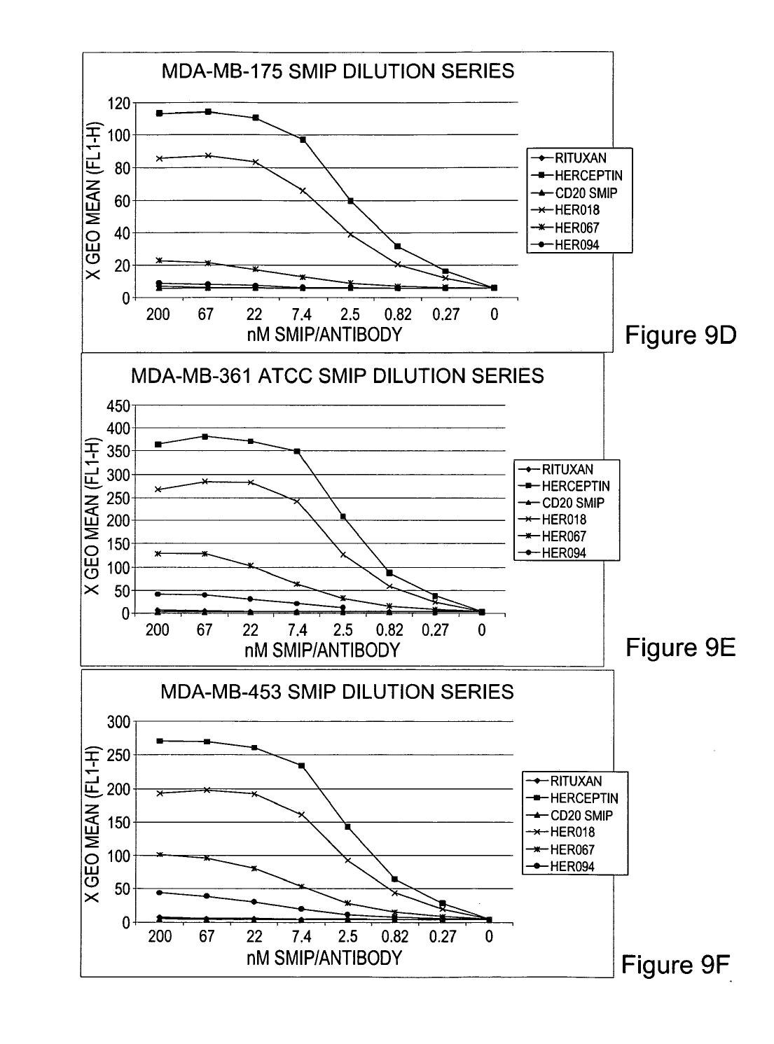

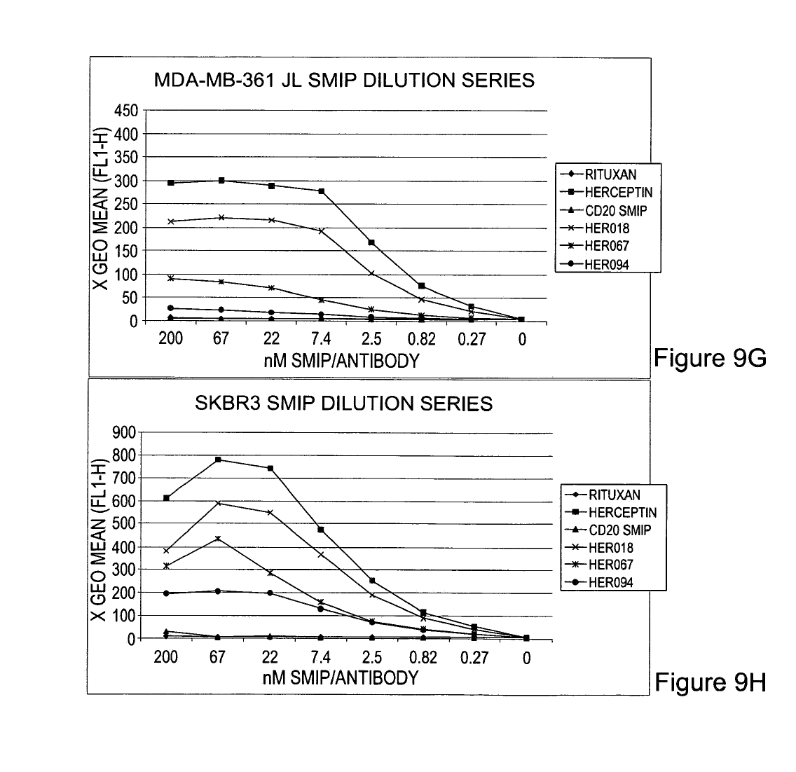

SMIPs maintain a similar staining pattern regardless of the amount of HER2 on the cell surface and the other ErbB receptors/ligands expressed by the cell lines (relative surface staining for ErbB1, Her2, Erb3 and production of ligand by cell lines is not shown). The SMIP/antibody staining pattern was Herceptin®>Her018>HER067 (Her033)>HER094 (Her030). The results of these binding assays are shown in FIG. 8 and FIG. 9A-9H. (In FIG. 9E, 0.82 nM HER094 data not collected due to mechanical error.)

### Example 5

### PCR Amplification of scFv Regions for Sequencing Analysis

PCR amplification of scFvs was carried out using the KOD HOT START DNA Polymerase kit (Novagen) in accordance with the manufacturers instructions. 0.2 μM each of the M13rev (5′ GGAAACAGCTATGACCATGA 3′) (SEQ ID NO: 247) forward and Mycseq (5′ CTCTTCTGAGATGAGTTTTTG 3′) (SEQ ID NO: 248) reverse primers were used. 5 μl of a 1:10 dilution of a stationary phase bacterial culture was used as the template for a final reaction volume of 20 μl. The cycling conditions used were a 2 minute hot start at 94° C., 25 cycles of denaturation at 94° C. (1 minute), primer annealing at 42° C. (30 seconds) and extension at 72° C. (1 min), followed by a final 5 minute extension at 72° C. PCR products were verified by agarose gel electrophoresis and cleaned up with Exol/SAP (shrimp alkaline phosphatase) prior to sequencing of both strands with primers 145837 (5′ GGAGATTTTCAACGTGAA 3′) (SEQ ID NO: 249) and 142051 (5′ CTCTTCTGAGATGAGTTTTTG 3′) (SEQ ID NO: 250). The closest human germlines of the V_{H} and V_{L} segments were determined (Table 4).

TABLE 4
V_{H} and V_{L} germlines of ERBB2 clones

| | Human V_{H} germline | Human V_{L} germline |
|---|---|---|
| Mab | gene | gene |
| S1R2A_CS_1F7 | 1-02 (DP8/75) | Vλ 3h |
| S1R2A_CS_1D11 | 1-69 (DP10) | Vλ 1b (DPL5) |
| S1R2C_CS_1D3 | 1-69 (DP10) | Vλ 1b (DPL5) |
| S1R2C_CS_1H12 | 3-48 (DP51) | Vλ 1c (DPL2) |
| S1R2A_CS_1D3 | 1-02 (DP8/75) | Vλ 1g (DPL3) |
| S1R3B2_BMV_1E1 | 3-33 (DP50) | Vλ 1b (DPL5) |
| S1R3C1_CS_1D3 | 6-1 (DP74) | Vλ 2c |
| S1R3B2_DP47_1E8 | 3-23 (DP47) | Vλ 1e (DPL8) |
| S1R3B2_BMV_1G2 | 1-18 (DP14) | Vκ L12 |
| S1R3B2_BMV_1H5 | 3-33 (DP50) | Vλ 2a2 (DPL11) |
| S1R3C1_CS_1A6 | 5-51 (DP73) | Vλ 1c (DPL2) |
| S1R3B2_DP47_1C9 | 3-23 (DP47) | Vλ 1c (DPL2) |
| S1R3B2_DP47_1E10 | 3-23 (DP47) | Vλ 1g (DPL3) |
| S1R3C1_CS_1B10 | 1-69 (DP10) | Vλ 6a |
| S1R3A1_BMV_1F3 | 3-21 (DP77) | Vλ 31 (DPL16) |
| S1R3B1_BMV_1G11 | 3-23 (DP47) | Vλ 2a2 (DPL11) |
| S1R3A1_BMV_1G4 | 1-03 (DP25) | Vλ 2a2 (DPL11) |
| S1R3B1_BMV_1H11 | 3-23 (DP47) | Vκ L12 |
| S1R3A1_CS_1B9 | 5-51 (DP73) | Vλ 8a (DPL21) |
| S1R3B1_BMV_1H9 | 4-04 (DP70) | Vλ 3l (DPL16) |
| S1R3A1_CS_1B10 | 1-02 (DP8/75) | Vλ 8a (DPL21) |
| S1R3B1_BMV_1C12 | 3-30.5 (DP49) | Vλ 1c (DPL2) |
| S1R3C1_BMV_1H11 | 3-33 (DP50) | Vλ 1e (DPL8) |
| S1R3B1_BMV_1A10 | 3-30.5 (DP49) | Vλ 3l (DPL16) |
| S1R3A1_CS_1D11 | 5-51 (DP73) | Vλ 8a (DPL21) |
| S1R3C1_DP47_1H1 | 3-23 (DP47) | Vλ 3h |
| S1R3A1_CS_1B12 | 1-02 (DP8/75) | Vλ 1e (DPL8) |
| S1R3B1_BMV_1H5 | 3-33 (DP50) | Vλ 3l (DPL16) |
| S1R3A1_DP47_1A6 | 3-23 (DP47) | Vλ 1c (DPL2) |
| S1R3B1_DP47_1E1 | 3-23 (DP47) | Vλ 6a |
| S1R3B1_BMV_1A1 | 1-18 (DP14) | Vλ 2a2 (DPL11) |

### Example 6

### BIACORE® Binding Assay

Binding of different Her2-directed binders (antibodies and SMIPs) to monomeric Her2 ECD and truncations of dimeric Her2 ECD were determined using a BIACORE® T100 instrument (GE Healthcare, Biacore, Piscataway, N.J.). Her2-directed binders were captured by a monoclonal mouse anti-human Fc (GE healthcare), which was covalently conjugated to a carboxylmethyl dextran surface (CM4) via amines using N-ethyl-N′-(3-dimethylaminopropyl)-carbodiimide hydrochloride and N-hydroxysuccinimide. The unoccupied sites of the activated surface were blocked by ethanolamine. The capturing antibody (referred to as anti hFc) binds to the C_{H}2 domain of IgG Fc of all sub-classes and showed no discernible dissociation from the captured her2-binders during the course of the assay. Every cycle, 3 different Her2 binders and a non-binder (negative control) were individually captured by anti hFc on 4 different flow cells, typically to about 50 RU, followed by injection of the analyte (Her2 dimers and monomer) at a particular concentration for 10 minutes over all flow cells. The dissociation of the formed complexes were subsequently followed for 12 minutes. At the end of the cycle, the surface was regenerated gently using 3M MgCl₂ which dissociates protein bound to the capturing anti hFc antibody. Multiple such cycles were performed to study binding of different analytes at different concentrations, in the range of 0-300 nM, for each set of three Her2 binders captured. Her2 binders were reproducibly captured every cycle with CV not exceeding 1%. The binding was performed at 25° C. in 0.01 M HEPES pH 7.4, 0.15 M NaCl, 0.005% v/v SURFACTANT P20. Signal associated with binding to the negative control was used to subtract for bulk refractive changes. The kinetic parameters and affinities were determined using BIAEVALUATION software.

HERCEPTIN® (trastuzumab) bound monomeric EQR, dimeric ECD and shed ECD (monomeric), weakly bound HER018 but did not bind a truncated fusion protein lacking the CR2 domain. In contrast, HER033 and HER030 bound only dimeric ECD and dimeric HER018 but did not bind monomeric EQR or shed ectodomain (ECD). Specifically for dimeric HER2 may be advantageous in that such binders may have increased selectivity for tumors and may not bind, or show reduced binding to tissues that express low levels of HER2 and/or where ligand independent homodimer formation is limited. Such HER2 binders with reduced binding to non-tumor target tissues (e.g., cardiac tissues) may, thus, have fewer side effects including lower toxicity. In addition, a lack of binding to shed HER2 ectodomain would reduce the effective dose compared to a HER2-binding agent that has significant binding to shed ECD.

The results of the BIACORE® assay are shown in FIG. 7.

Trastuzumab and the SMIP version of trastuzumab (HER018) bind full length dimer and monomer soluble receptors similarly at low nanomolar levels (about 1 to about 5 nM), whereas truncated dimer soluble receptors (i.e., lacking all three trastuzumab contact sites) are bound poorly or not at all (see Table 5). In contrast, Her030 and Her033/Her067 SMIPs bind soluble dimer receptors at nanomolar affinities (about 4 to about 8 nM), but not monomer HER2. The HER033 and HER067SMIPs have the same amino acid sequence, but the difference between them is that the former is produced in HEK cells while the latter is produced in CHO cells. Binding by HER033 and HER067SMIPs is substantially the same. HER030 appears to bind less strongly than Her033/Her067 to the dimers.

TABLE 5
BIACORE ® binding affinity summary

| | Affinity (nM) at 25° C. | | | | |
|---|---|---|---|---|---|
| | | Her | Her | Her | Her |
| | Herceptin | 018 | 033 | 067 | 030 |
| SIIS (Dimer) | 1.06 | 1.4 | 7.23 | 8.18 | 35.6 |
| 1.8 (Dimer) | 228 | 167 | 4.92 | 6.47 | 27.6 |
| 1.6 (Dimer) | NB | NB | NB | NB | NB |
| SIIS (Monomer) (Her155) | 3.44 | 4.59 | 508 | ND | ND |

NB—No Binding Observed
ND—not enough binding to fit

### Example 7

### BrdU and ATP Proliferation Assays

To 96-well plates, cells were added at 2.5×10³ cells/well (SKBR3, BT474, MDA-MB-453, MDA-MB-175) or at 5×10³ cells/well (MDA-MB-361). The next day, SMIPs were added to the cells at the desired concentration and then incubated at 37° C./5% CO₂ for 4 (SKBR3, MDA-MB-453, MDA-MB-361, MDA-MB-175), 5 (BT474), or 7 (MDA-MB-361) days. The day before cells were harvested, 5-bromo-2′-deoxyuridine (BrdU) is added to a final concentration of 0.1 mM and continued to incubate overnight at 37° C. After incubation, media was removed and then the cells were treated with ethanol-based fix solution (DELFIA® Cell Proliferation Kit, Perkin Elmer, Waltham, Mass.) at room temperature (RT) for 30 minutes. Fix solution was removed by aspiration, 100 μl/well anti-BrdU-Eu labeled antibody (0.5 mg/mL) was added, and the cells were incubated at RT for 2 hours. Cells were then washed 4 times with Tris-based DELFIA Platewash (300 μl/well/wash). DELFIA Inducer (with Triton® X-100, glycine, HCl, and chelator) was then added to the cells (200 μl/well) and incubated with shaking for 15 minutes at RT. Fluorescence was measured using Flex Station® 3 in Time resolved fluorescence mode (Molecular Devices, Sunnyvale, Calif.).

After the proliferation assay fluorescence reading, the DELFIA Inducer was removed by aspiration and Hoechst 33342 nuclear stain solution (Invitrogen, Carlsbad, Calif.) was added to the cells. Nuclear stain fluorescence was measured on an IN Cell Analyzer at 4× resolution.

Alternatively, we investigated anti-Her2 SMIP anti-proliferation activity in MDA-MB-361 cells as follows. MDA-MB-361 breast cancer cells were plated in 96-well format and treated with anti-Her2 or control reagents for indicated concentrations and times (24-96 hr). For proliferation assays, media (DMEM plus 10% FBS) was removed, the cells washed with phosphate-buffered saline (PBS), fixed with 4% paraformaldehyde and nuclei stained with DAPI (Molecular Probes). Stained nuclei were counted using Cellomics High Content assay measuring fluorescence at 360 nM. For apoptosis assay, fixed cells were permeabilized by treatment with 0.2% Triton 100 in PBS prior to primary staining with mouse anti-cleaved PARP antibody (Cell Signaling Technologies) and secondary staining with goat anti-mouse IgG labeled with ALEXA488 (Invitrogen). Fluorescence was measured in Cellomics High Content assay at 488 nM.

ATP Lite First Step assay (Perkin Elmer) was used to assess cellular viability by measuring ATP levels via luminescence (ATP luciferase). To 96-well plates, cells were added at 2.5×10³ cells/well (SKBR3, BT474, MDA-MB-453, MDA-MB-175) or at 5×10³ cells/well (MDA-MB-361). The next day, SMIPs were added to the cells at the desired concentration and then incubated at 37° C./5% CO₂ for 4 (SKBR3, MDA-MB-453, MDA-MB-361, MDA-MB-175), 5 (BT474), or 7 (MDA-MB-361) days. After SMIP incubation for the desired amount of time, lyophilized ATP Lite substrate is reconstituted with 10 ml of ATP Lite substrate/lysis solution and allowed to sit at room temperature for 10 minutes. This reconstituted substrate solution was added to the cells (100 μl/well) and read luminescence on Top Count Reader (Packard).

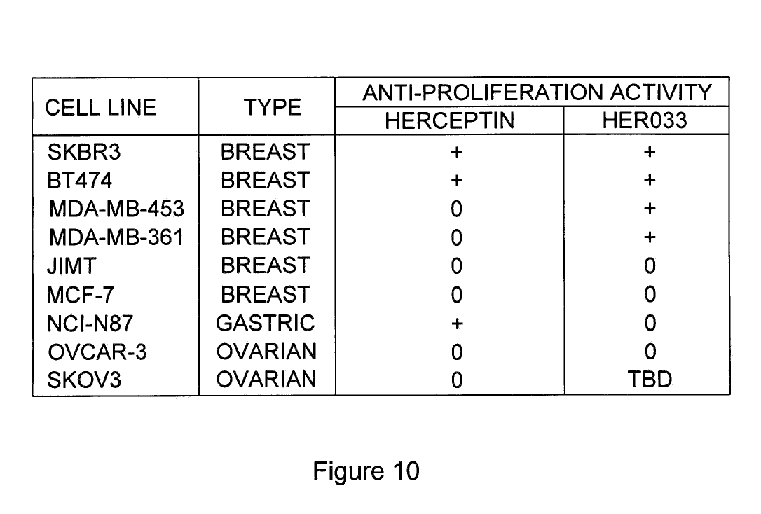

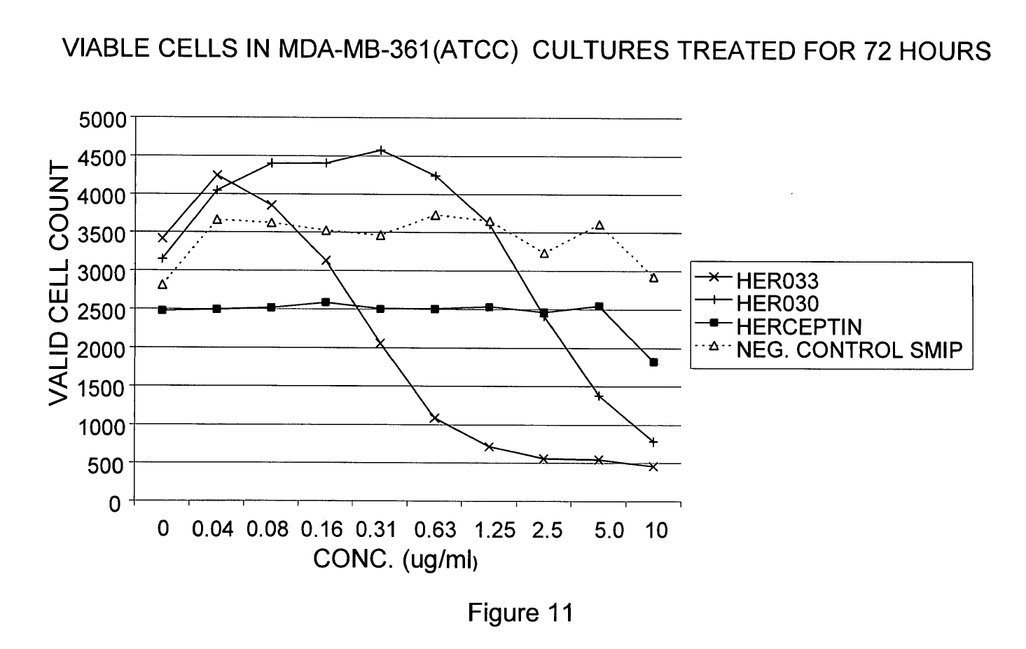

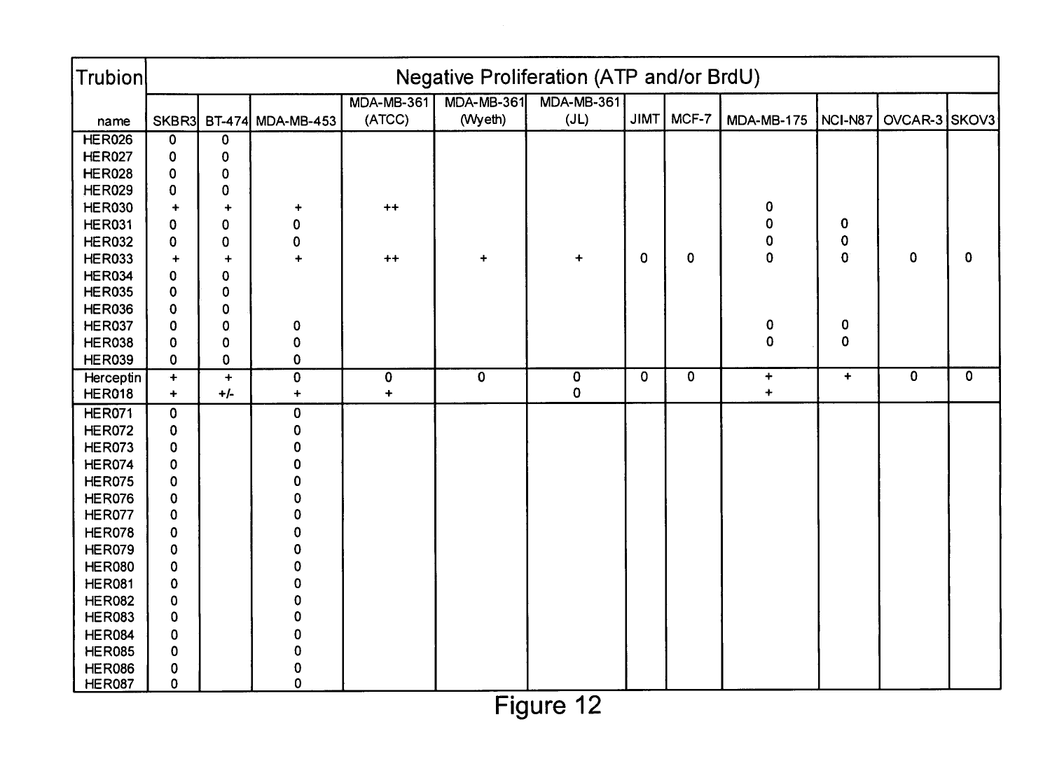

The results of the proliferation assays are shown in FIGS. 10-12.

### Example 8

### Pathway Phosphorylation Assays

To 96-well plates, cells were added at 8−12×10³ cells/well depending on cell type (Becton-Dickinson, San Jose, Calif.) and allowed to incubate overnight in growth medium with serum at 37° C./5% CO₂. After removal of growth medium, the cells were washed with serum-free medium, aspirated, and then serum-free media was added for incubation at 37° C./5% CO₂ for 3 hours. The SMIP of interest was prepared in prewarmed serum-free media, added to each well at the indicated concentration, and incubated at 37° C./5% CO₂ for desired time points. As a control, signaling was inhibited with AG825 (Calbiochem, LaJolla, Calif.) at 40 μM; LY294002 (Cell Signaling) at 50 μM; or 00126 MEK1/2 inhibitor (Cell Signaling) at 10 μM. The cells were then fixed in formaldehyde (diluted in 1×PBS) at a final concentration of 3.7% for 10 minutes at 37° C./5% CO₂. The cells were then washed two times with PBS. After removing the PBS, the cells were permeabilized in 0.1% Triton® X-100 (Sigma-Aldrich, St. Louis, Mo.) solution diluted in 1×PBS at room temperature for 5 minutes. The cells were then washed two times with PBS and blocked by incubation in PBS/1% BSA (Sigma-Aldrich) at room temperature for 30 minutes (or overnight at 4° C.).

The blocking solution was removed and primary antibody (in PBS with 3% horse serum or PBS with 1% BSA, and 0.1% Triton® X-100) was added for 1 hour at room temperature (or overnight at 4° C.). The primary antibodies used (at 0.125 μg/well) were (1) rabbit anti-phospho-akt (Ser473) (Cell Signaling, Danvers, Mass.); (2) mouse anti-phospho-Erk1/2 (Cell Signaling, Danvers, Mass.); and (3) rabbit anti-phospho-ErbB2 (Abgent, San Diego, Calif.). The primary antibody was removed and the cells were washed 3 times with PBS. The secondary antibody (in PBS with 3% horse serum or PBS with 1% BSA, and 0.1% Triton® X-100) was then added for 1 hour at room temperature (or overnight at 4° C.) protected from light. The secondary antibodies used (at 0.2 μg/well) were Alexa 488 donkey anti-rabbit IgG (Invitrogen, Carlsbad, Calif.) and DyLight 649 goat anti-ms IgG (Pierce, Rockford, Ill.). The secondary antibody was removed and the cells were washed 3 times with PBS. Then 100 μL of PBS containing 200 ng/ml Hoechst 33342 nuclear stain (Invitrogen, H3570) (and if needed 1 ug/ml CellMask Blue cytoplasmic stain (Invitrogen, H34558) was added to the cells. The plates were covered and kept protected from light. The plates were then imaged.

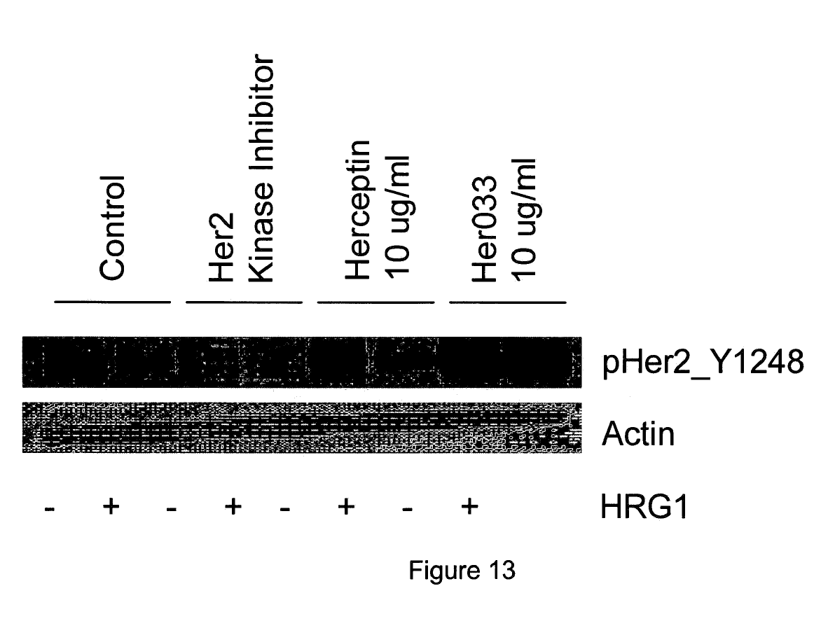

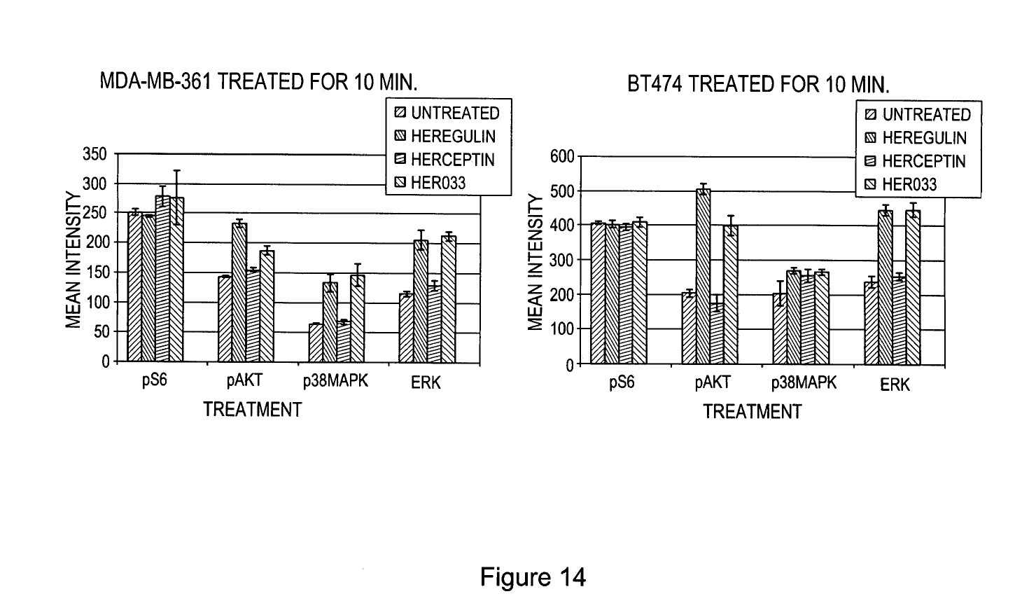

Alternatively, we investigated anti-Her2 SMIP signal transduction activity in MDA-MB-361 cells as follows. MDA-MB-361 breast cancer cells, were plated in 6-well plate to 80-90% confluency (DMEM plus 10% FBS) and treated with anti-Her2 or control reagents for 24 hr with and without pretreatment with Heregulin (HRG—15 min.) or EGF (30 min.). For assay of total and phosphorylated Her2, cells were lysed, 50 ug total protein was fractionated using SDS-PAGE and transferred to nitrocellulose membranes using standard procedures. Western blot analysis used either rabbit anti-Her2 antibody (Cell Signaling Technologies), anti-pHer2_Y1248 (Upstate) or anti-Actin (Santa Cruz) as primary antibody and subsequently stained with HRP-conjugated anti-rabbit IgG. Peroxidase activity was measured using ECLplus2 kit (GE Healthcare) following manufacturer's protocols and exposed to film. As shown in FIG. 13, HER033 induces HER2 phosphorylation.

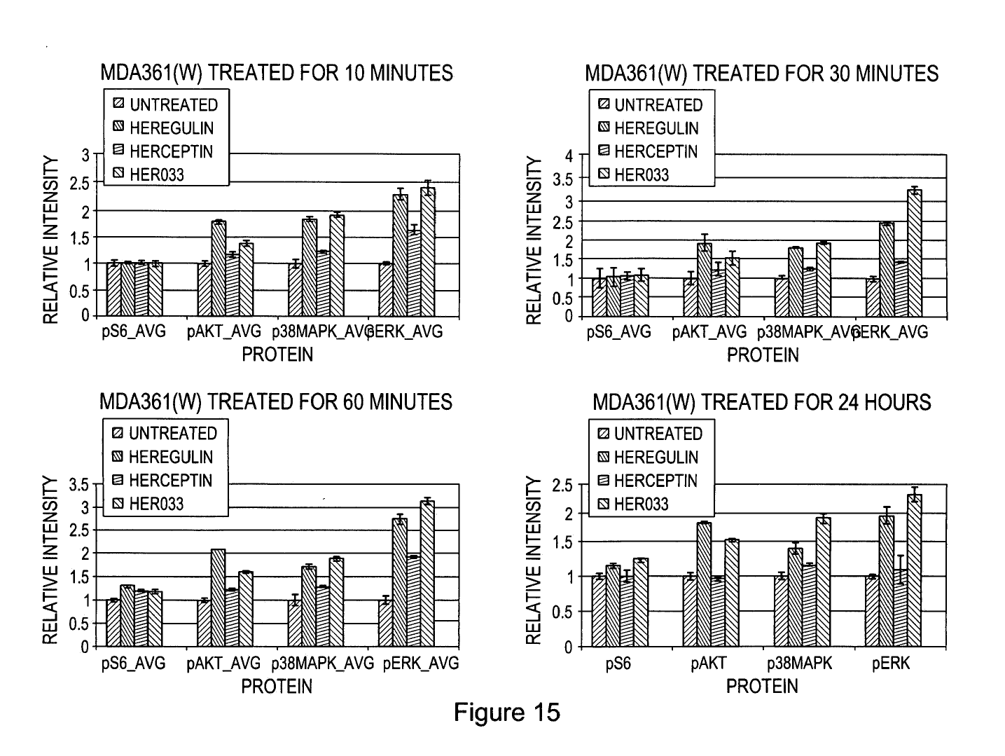

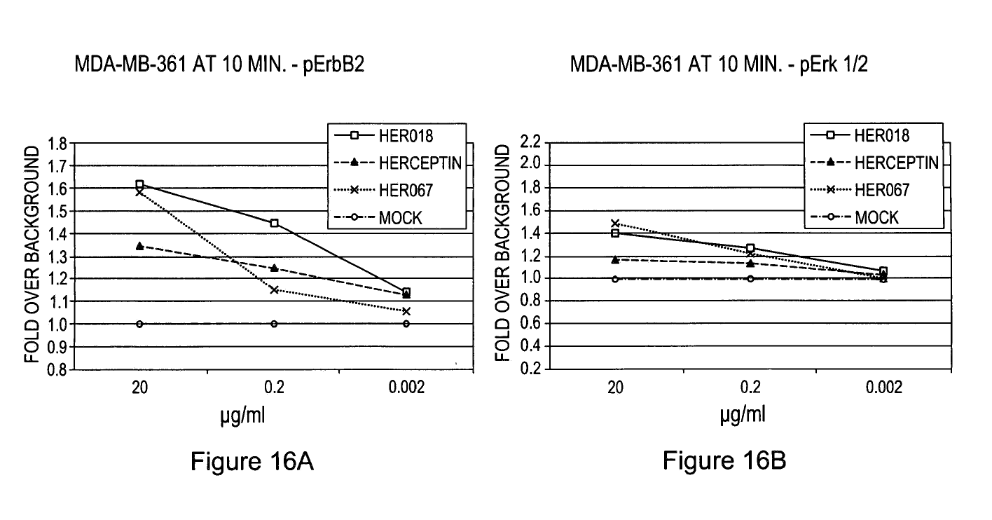

To measure increased downstream phosphoprotein signal transduction, MDA-MB-361 breast cancer cells were plated in 96-well format and treated with anti-Her2 or control reagents for the concentrations and times (10 min to 24 hr) shown in FIG. 15. Media was removed, cells washed with PBS, fixed with 4% paraformaldehyde, and permeabilized with 0.2% Triton 100/PBS. Cells were subsequently stained with either rabbit anti-pAKT (Cell Signaling Technologies), anti-pERK (Cellomics), anti-pS6K (Cell Signaling Technologies), or anti-p38MAPK (Cell Signaling Technologies). Following PBS wash (3×), cells were stained with secondary goat anti-rabbit IgG antibody labeled with ALEXA594. Cell fluorescence was quantified using Cellomics High Content assay at 594 nM.

Her067 (Her033) has agonistic activity (increased signaling) compared to trastuzumab (see Table 6). Moreover, Her067 and Her018 are generally a stronger inducer of Her2, Erk1/2, and Akt phosphorylation than trastuzumab. The increase was statistically significant as compared to the mock treatment when measured by the pairwise student T-test (<0.001).

TABLE 6
Induction of phosphorylation by HER018,
HER067, Herceptin and Heregulin

| MDA-MB-361(JL) | HER018 | HER067 | Herceptin | Heregulin |
|---|---|---|---|---|
| phospho-ErbB2 | ++ | ++ | + | + |
| phospho-Erk1/2 | + | ++ | + | + |
| phospho-Akt | + | + | + | ++ |

### Example 9

### Cell Cycle Assay

To investigate the effect of the ErbB2 ECD binder on cell cycle in HERCEPTIN® sensitive and HERCEPTIN® resistant cells, each well of a 6 well plate was seeded with 2×10⁵ cells (SKBR3 or BT474 (sensitive) or MDA-MB-453 or MDA-MB-361 (resistant) and incubated overnight at 37° C./5% CO₂. Cells were then treated with antibody or SMIP (at 10 μg/ml final) (in triplicate) and incubated for another 24 or 48 hours. After incubation, the cells were pulsed with 50 uM BrdU (Sigma) for 30 minutes at 37° C., the media was removed, and the cells were treated with trypsin and harvested in a FACS tube on ice. The cells were washed with PBS, fixed with 70% cold ethanol, and incubated on ice for 30 minutes. The ethanol was removed and then 2N HCl/0.5% Triton X-100 was added, and the cells were incubated for 30 minutes at room temperature (RT). The acid was removed and neutralized with 0.1 M Na₂B₄O₇ for 15 min at RT. The neutralization buffer was removed, FITC labeled anti-BrdU antibody was added (BD Bioscience) in PBS/0.5% TWEEN® 20/1% BSA, and the cells were incubated for 30 minutes at RT in the dark. The FITC dye was removed, the cells washed, and then DAPI nuclear stain (Invitrogen) and RNAse A (Qiagen) each at 1:1000 dilution was added and the cells were incubated 15 minutes in the dark and then analyzed by FACS. Statistical analysis of the data was performed using ANOVA and Student's t-test.

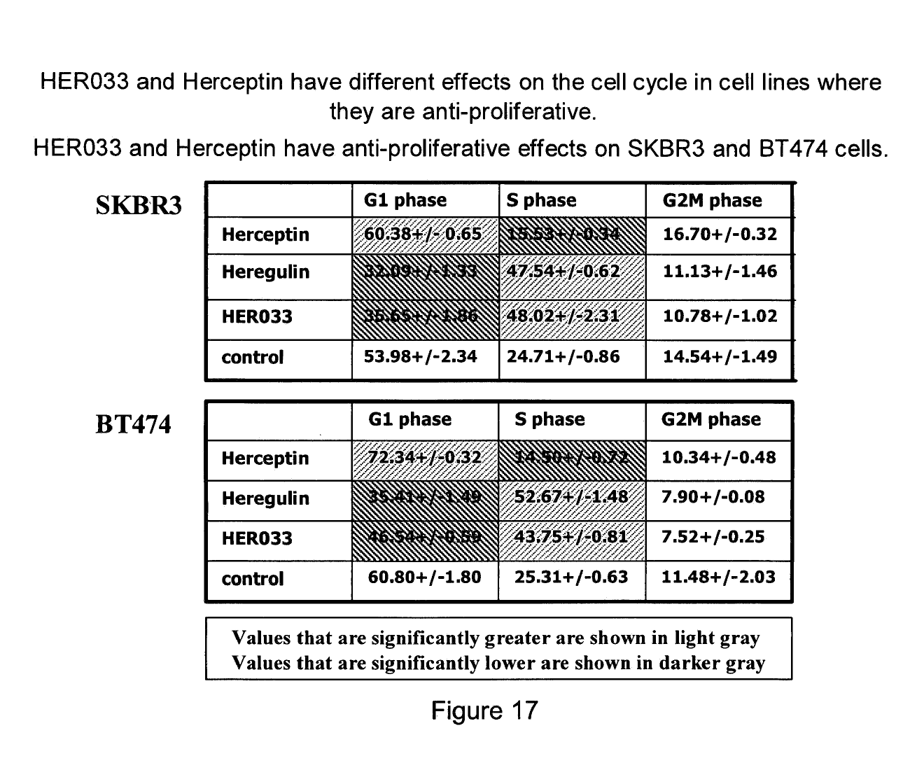

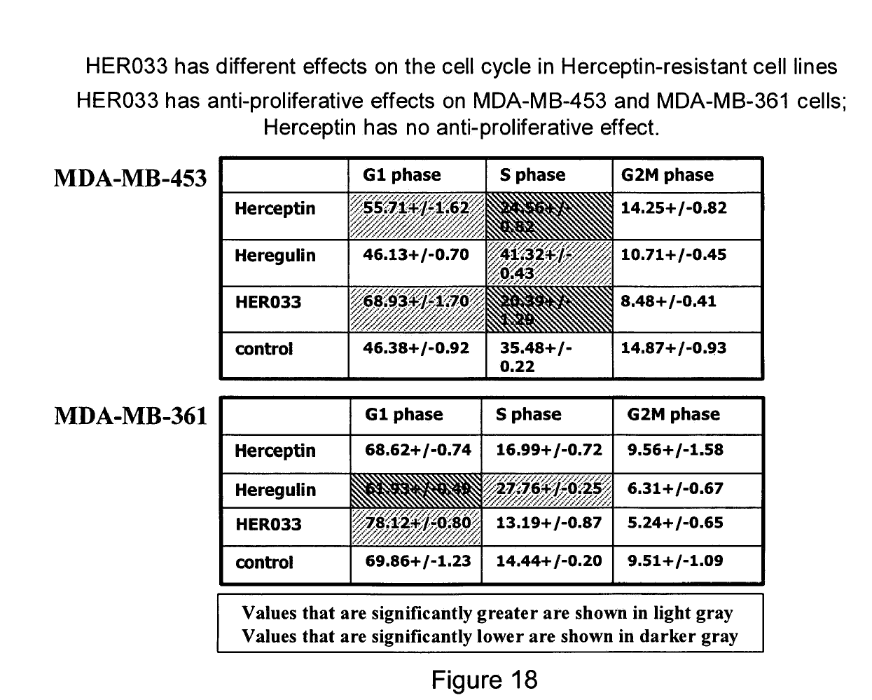

The results are presented in FIGS. 17 and 18. We observed an increased number of cells in the G1 phase in HERCEPTIN® treated SKBR3, BT474 and MDA-MB-453 cells. Among cells treated with HER033SMIP, we observed an increased number of cells in S phase in SKBR3 and BT474 cells.

### Example 10

### In Vivo Xenograft Assay

To investigate the effect of the ErbB2 binding molecules of the invention in vivo, we tested the molecules in three mouse models.

SCID/Beige Mouse Model

Female (6-7 week old) Beige SCID mice (Beige SCID CB-17/IcrHsd-Prkdcscid-Lystbg) were obtained from Harlan Sprague Dawley, N.J. Virus free MDA-MB-361 cells were thawed from a new vial and cultured to generate appropriate numbers. Cells were grown to near confluency and had a viability of >90%. Cells were harvested, washed twice with sterile PBS, resuspended to 2×10⁸ cells/ml, then combined with Matrigel 1:2. and kept on ice until injection.

Tumor Cell Implantation and Monitoring: Each mouse was injected with 100 μl of the cell/Matrigel suspension (1×10⁷ cells) subcutaneously on the right flank. Mice were monitored daily for tumor growth. Tumors were established when they reached about 150 to about 300 mm³ (Volume=½[length×(width)²). Tumors developed in 100% of the implanted mice. Mice were sorted into groups according to tumor size, keeping means consistent among groups using LabCat software. Sorting occurred on day 0, which was the same day the mice received their first treatment.

Mice were monitored (i.e., weighed and tumors measured) two to three times weekly. Mice were sacrificed if ulceration of tumor occurred, extreme body weight loss (greater than or equal 20%), tumor exceeded about 1200 to about 1500 mm³, or tumor inhibited mobility of a mouse. The study is continued for a total of about 60 days.

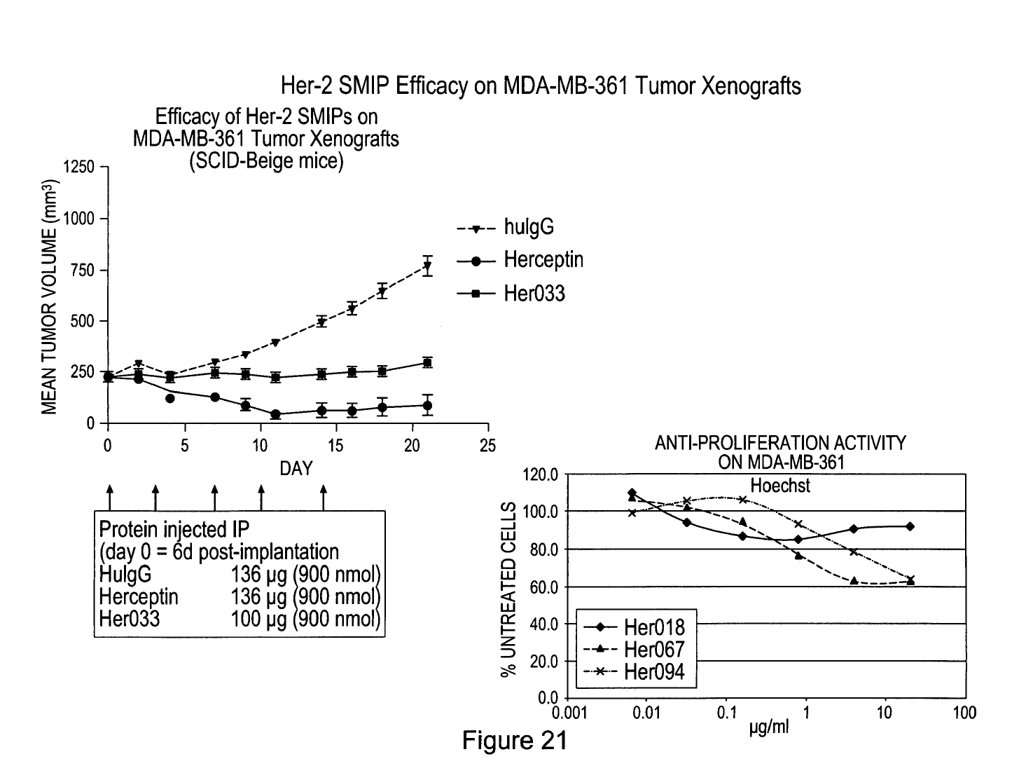

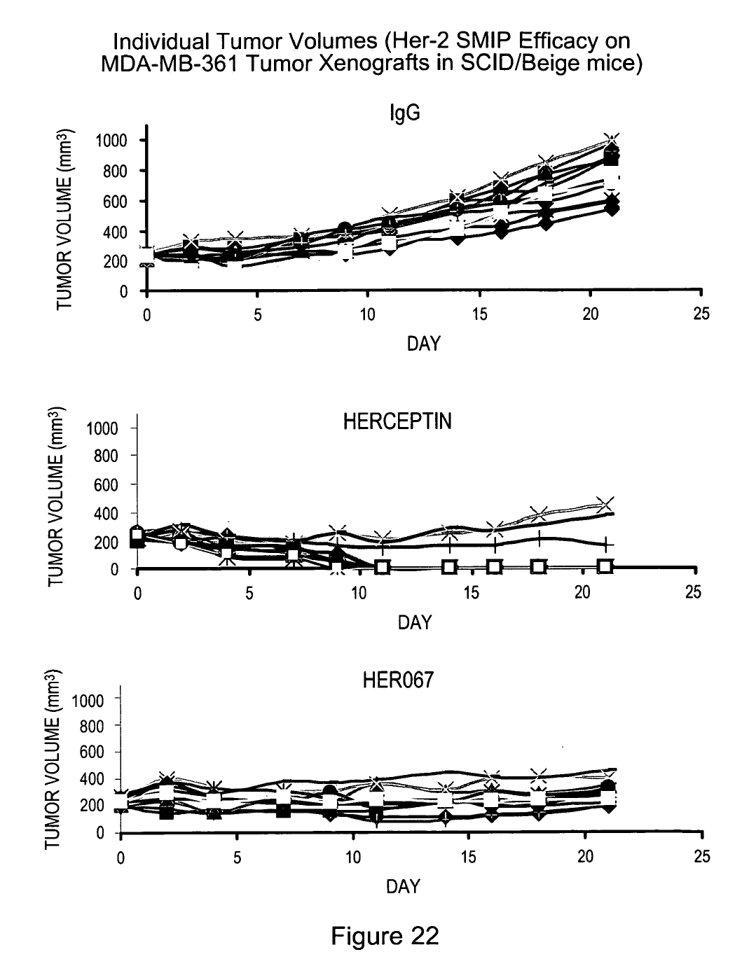

Treatment: Mice were sorted into three groups of 11 mice each. Treatment began on day 0 (about six days after cell implantation). Each mouse of a group received intraperitoneal treatments twice a week (for a total of five treatments), which were given in equimolar amounts (900 nM) of (1) SMIP HER067 (100 μg), (2) Herceptin (136 μg, positive control), or (3) human IgG (136 μg, negative control). Survival and tumor size was recorded two to three times weekly. Results were graphed (+/−SEM) and analyzed using Prism software (see FIGS. 21 and 22).

BALB/c nu and nu/nu Mouse Models

Male BALB/c nu/nu (nude) mice (18-23 g) and female nu/nu (nude) mice (18-23 g) were obtained from Charles River Laboratories, Wilmington, Mass.

Subcutaneous BCL Xenografts:

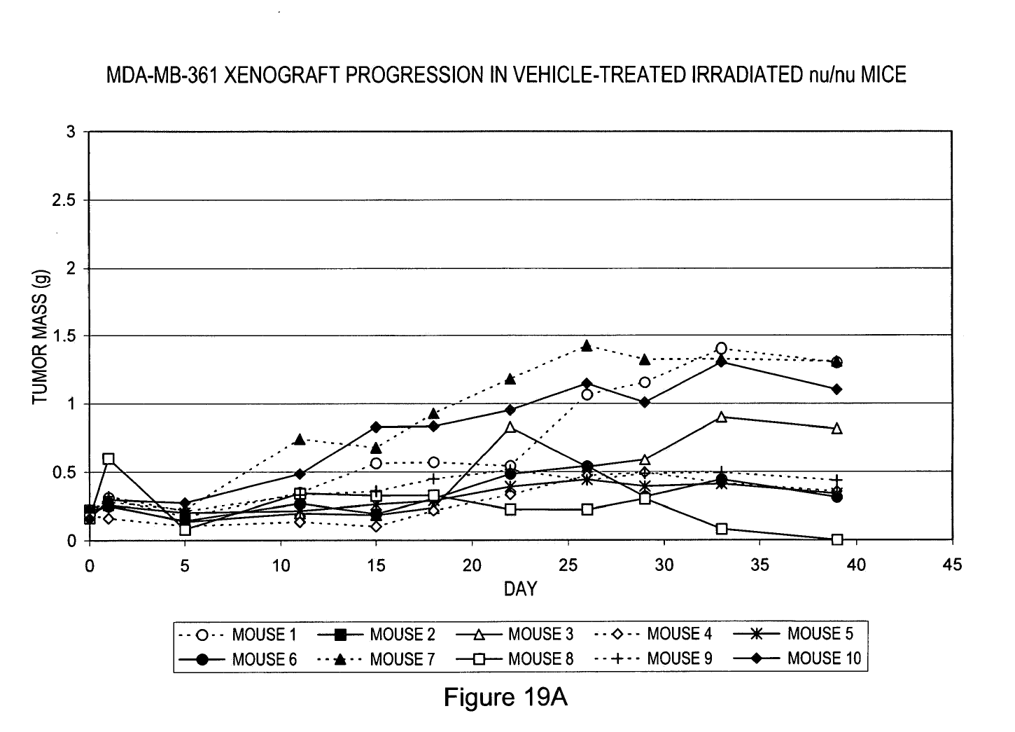

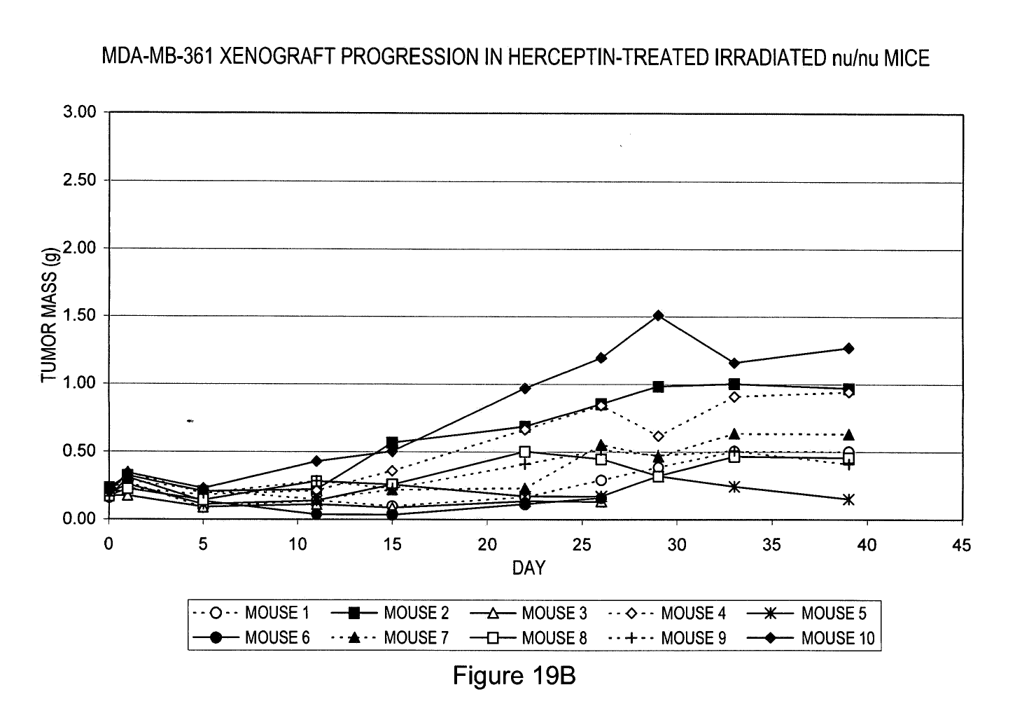

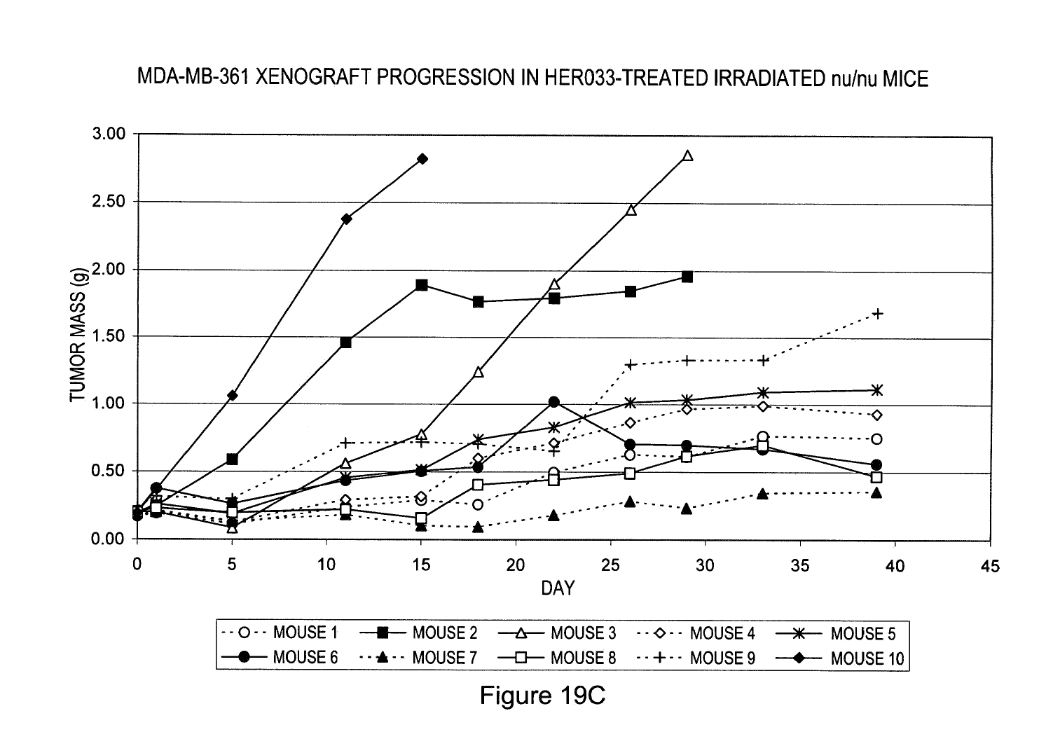

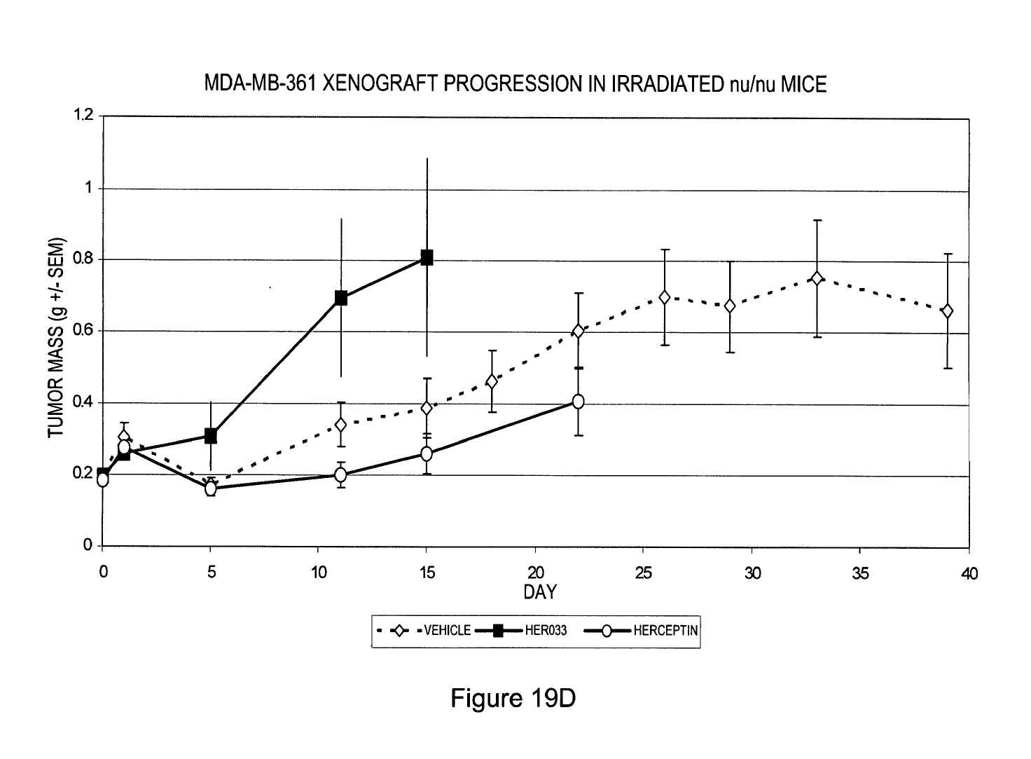

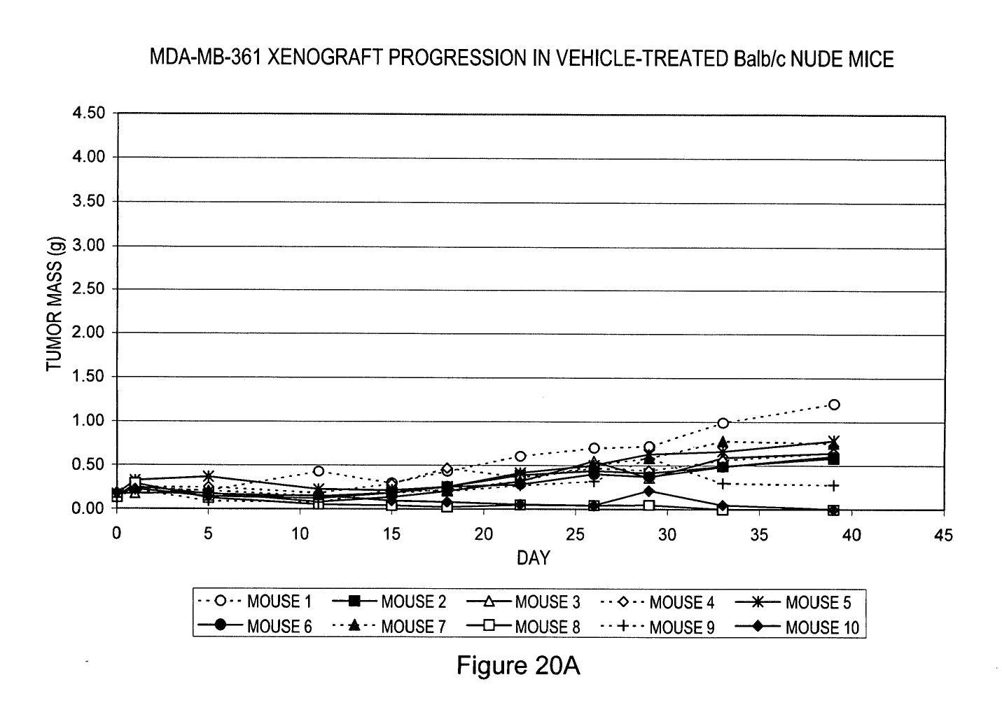

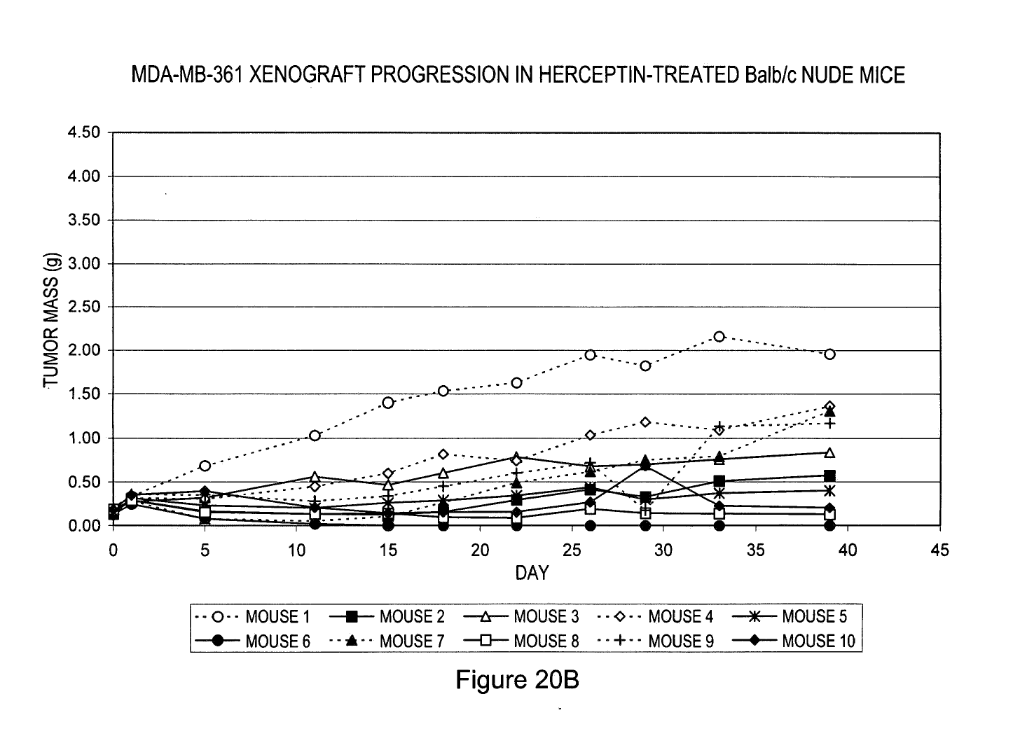

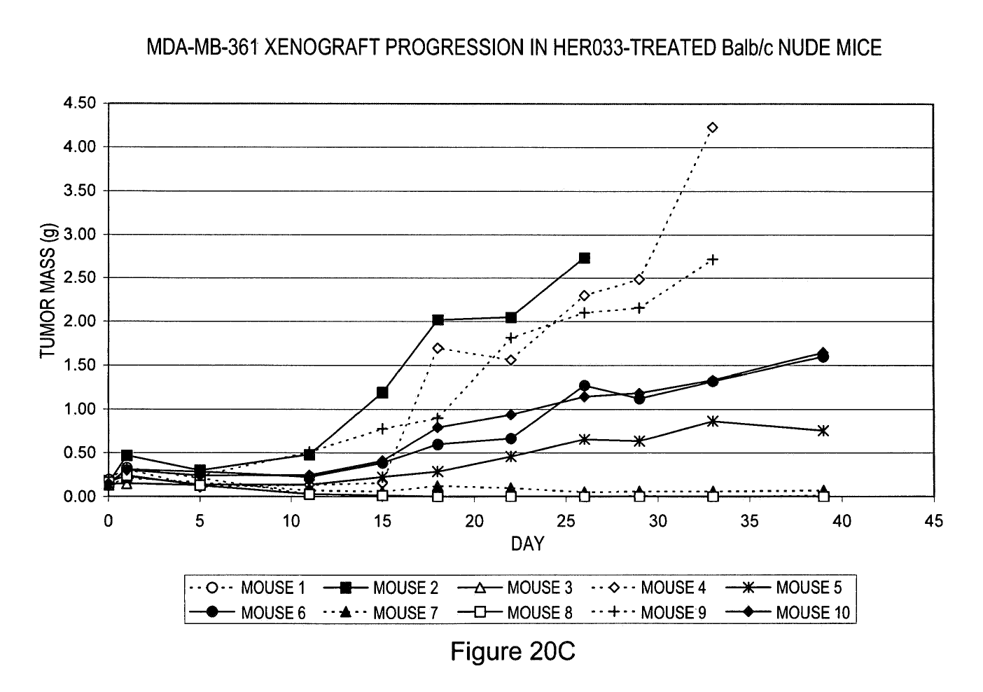

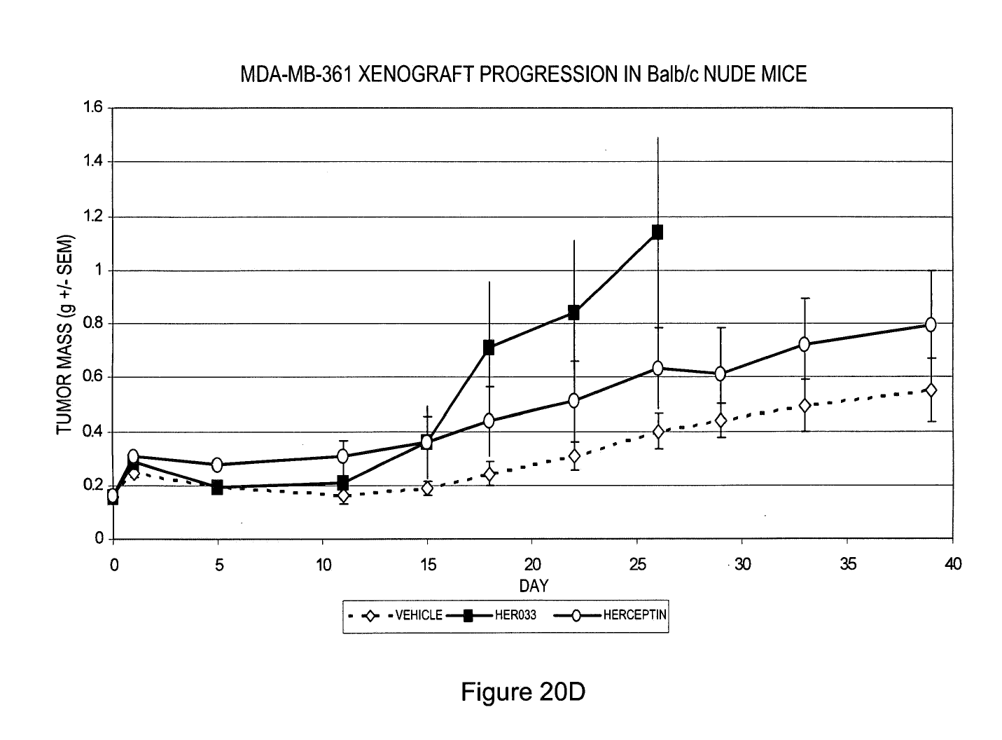

Female, athymic nude mice were exposed to total body irradiation (400 rads) to further suppress their residual immune system and facilitate the establishment of xenografts. Three days later, the irradiated mice were injected subcutaneously (SC) with 1×10⁷ MDA-MB-361 cells in Matrigel (Collaborative Biomedical Products, Belford, Mass., diluted 1:1 in culture medium) in the dorsal, right flank. When the tumors reached the mass of 0.1 to 0.25 g, the tumors were staged to ensure uniformity of the treatment groups. Male, athymic Balb/c nude mice were injected s.c. with 1×10⁷ cells in the right flank. When tumors reached an average tumor mass of 0.1 to 0.25 g, the tumors were staged to ensure uniformity of the treatment groups. Mice were dosed with compounds (100 μg/mouse ip) on days 1,4,6,8 and 11 (n=10 mice/treatment group). All compounds were administered ip. Tumors were measured at least once a week and their mass (±SEM) was calculated. Tumor mass for each treatment group was compared to that from the vehicle-treated group for statistical significance using ANOVA and subsequent pairwise comparisons to the vehicle-treated group using a one-tailed t-test with the error term for the t-test based on the pooled variance across all treatment groups. The results are shown in FIGS. 19 and 20.

The preliminary results in vivo as shown in FIGS. 19-22 are inconclusive. A number of factors could contribute to the differences observed in the three mouse models and are being further investigated. For example, while not intending to be limiting, the different experiments were dosed differently (twice weekly as compared to every other day, which means the former dosing lasted over a longer period of time, the tumors in the vehicle control groups in some of the experiments did not grow particularly well, and the mouse backgrounds had differing effector functionality (i.e, the nu/nu nude mice have B cells and NK cells, while the SKID/Beige mice have macrophages and monocytes. Based on the in vitro and in vivo results taken as a whole, the anti-ErbB2 binding proteins are believed to be efficacious in treating tumors.

The specification is most thoroughly understood in light of the teachings of the references cited within the specification. The embodiments within the specification provide an illustration of embodiments of the invention and should not be construed to limit the scope of the invention. The skilled artisan readily recognizes that many other embodiments are encompassed by the invention. All publications and patents cited in this disclosure are incorporated by reference in their entirety. To the extent the material incorporated by reference contradicts or is inconsistent with this specification, the specification will supercede any such material. The citation of any references herein is not an admission that such references are prior art to the present invention.

Unless otherwise indicated, all numbers expressing quantities of ingredients, reaction conditions, and so forth used in the application, are to be understood as being modified in all instances by the term “about.” Accordingly, unless otherwise indicated to the contrary, the numerical parameters are approximations and may vary depending upon the desired properties sought to be obtained by the present invention. At the very least, and not as an attempt to limit the application of the doctrine of equivalents, each numerical parameter should be construed in light of the number of significant digits and ordinary rounding approaches.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein.

What is claimed is:

1. A binding protein that specifically binds ErbB2, wherein the binding protein is an ErbB2 agonist.

2. The binding protein of claim 1 which reduces cellular proliferation in an ErbB2-expressing cancer cell.

3. The binding protein of claim 1 or claim 2 which increases apoptosis in an ErbB2-expressing tumor.

4. The binding protein of claim 1 which reduces the growth of an ErbB2-expressing tumor.

5. The binding protein of claim 2 wherein the ErbB2-expressing cancer cell is a breast cancer cell.

6. The binding protein of claim 2 wherein the ErbB2 expressing cancer cell is from a cell line selected from the group consisting of: SKBR3, BT474, MDA-MB-453 and MDA-MB-361.

7. A binding protein that specifically binds ErbB2, wherein the binding protein preferentially binds an ErbB2 extracellular domain (ECD) homo-dimer over ErbB2 ECD monomer and shed ErbB2 ECD.

8. The binding protein of claim 1, wherein the binding protein preferentially binds an ErbB2 extracellular domain (ECD) homo-dimer over ErbB2 ECD monomer and shed ErbB2 ECD.

9. The binding protein of claim 2 or claim 7, that possesses one or more or the following properties:
(a) increases ErbB2 phosphorylation in a breast cancer cell;
(b) increases the phosphorylation of one or more of AKT, MAPK and ERK; or
(c) binds ErbB2 ECD in the CR2 domain.

10. The binding protein of claim 1 which is an antibody, an antigen-binding fragment of an antibody or a small modular immunopharmaceutical (SMIP).

11. The binding protein of claim 10 which is an antigen-binding fragment of an antibody, wherein the antigen-binding fragment is selected from the group consisting of: a Fab fragment, an F(ab′)2 fragment, an scFv, a dAb, and Fv fragment and a VHH.

12. The binding protein of claim 1, which is human antibody or an antigen-binding fragment thereof.

13. The binding protein of claim 1, wherein the ErbB2 is human ErbB2 (SEQ ID NO: 246).

14. A binding protein that specifically binds ErbB2, wherein the binding protein comprises:
(a) a VH domain comprising the CDR1, CDR2 and CDR3 amino acid sequences set forth in any one of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 65 or 67; or
(b) a VL domain comprising the CDR1, CDR2 and CDR3 amino acid sequences set forth in any one of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 63, 64, 66, 68, 69, 70, 71, 72, 73, 74, 75, 76, 77, 78, 79, 80, 81, 82, 83, 84, 85, 86, 87, 88, 89, 90, 91, 92, 93, 94 or 95; or
(c) a VH of (a) and a VL of (b).

15. The binding protein of claim 14, comprising the VH CDR1, CDR2 and CDR3 amino acid sequences and the VL CDR1, CDR2 and CDR3 sequences of any one of: S1R2A_CS_{—}1F7, S1R2A_CS_{—}1D11, S1R2C_CS_{—}1D3, S1R2C_CS_{—}1H12, S1R2A_CS_{—}1D3, S1R3B2_BMV_{—}1E1, S1R3C1_CS_{—}1D3, S1R3B2_DP47_{—}1E8, S1R3B2_BMV_{—}1G2, S1R3B2_BMV_{—}1H5, S1R3C1_CS_{—}1A6, S1R3B2_DP47_{—}1C9, S1R3B2 DP47_{—}1E10, S1R3C1_CS_{—}1B10, S1R3A1_BMV_{—}1F3, S1R3B1_BMV_{—}1G11, S1R3A1_BMV_{—}1G4, S1R3B1_BMV_{—}1H11, S1R3A1_CS_{—}1B9, S1R3B1_BMV_{—}1H9, S1R3A1_CS_{—}1B10, S1R3B1_BMV_{—}1C12, S1R3C1 BMV 1H11, S1R3B1_BMV_{—}1A10, S1R3A1_CS_{—}1D11, S1R3C1_DP47_{—}1H1, S1R3A1_CS_{—}1B12, S1R3B1_BMV_{—}1H5, S1R3A1_DP47_{—}1A6, S1R3B1_DP47_{—}1E1 or S1R3B1_BMV_{—}1 A1.

16. A binding protein that specifically binds ErbB2, wherein the binding protein comprises:
(a) a VH having the amino acid sequence that is at least 90% identical to any one of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 65 or 67; or
(b) a VL having the amino acid sequence that is at least 90% identical to any one of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 63, 64, 66, 68, 69, 70, 71, 72, 73, 74, 75, 76, 77, 78, 79, 80, 81, 82, 83, 84, 85, 86, 87, 88, 89, 90, 91, 92, 93, 94 or 95;
(c) a VH of (a) and a VL of (b); or
(d) a VH and aVL amino acid sequence that are at least 90% identical to the VH and VL, amino acid sequences, respectively, in any one of S1R2A_CS_{—}1F7, S1R2A_CS_{—}1D11, S1R2C_CS_{—}1D3, S1R2C_CS_{—}1H12, S1R2A_CS_{—}1D3, S1R3B2_BMV_{—}1E1, S1R3C1_CS_{—}1D3, S1R3B2_DP47_{—}1E8, S1R3B2_BMV_{—}1G2, S1R3B2_BMV_{—}1H5, S1R3C1_CS_{—}1A6, S1R3B2_DP47_{—}1C9, S1R3B2_DP47_{—}1E10, S1R3C1_CS_{—}1B10, S1R3A1 BMV 1F3, S1R3B1_BMV_{—}1G11, S1R3A1_BMV_{—}1G4, S1R3B1_BMV_{—}1H11, S1R3A1_CS_{—}1B9, S1R3B1_BMV_{—}1H9, S1R3A1_CS_{—}1B10, S1R3B1_BMV_{—}1C12, S1R3C1_BMV_{—}1H11, S1R3B1_BMV_{—}1A10, S1R3A1_CS_{—}1D11, S1R3C1_DP47_{—}1H1, S1R3A1_CS_{—}1B12, S1R3B1_BMV_{—}1H5, S1R3A1_DP47_{—}1A6, S1R3B1_DP47_{—}1E1 or S1R3B1_BMV_{—}1A1.

17. The binding protein of claim 16, wherein the binding protein comprises:
(a) a VH having the amino acid sequence that is at least 95% identical to any one of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 65 or 67; or
(b) a VL having the amino acid sequence that is at least 95% identical to any one of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 63, 64, 66, 68, 69, 70, 71, 72, 73, 74, 75, 76, 77, 78, 79, 80, 81, 82, 83, 84, 85, 86, 87, 88, 89, 90, 91, 92, 93, 94 and 95; or
(c) a VH of (a) and a VL of (b); or
(d) a VH and aVL amino acid sequence that are at least 95% identical to the VH and VL, amino acid sequences, respectively, in any one of S1R2A_CS_{—}1F7, S1R2A_CS_{—}1D11, S1R2C_CS_{—}1D3, S1R2C_CS_{—}1H12, S1R2A_CS_{—}1D3, S1R3B2_BMV_{—}1E1, S1R3C1_CS_{—}1D3, S1R3B2_DP47_{—}1E8, S1R3B2_BMV_{—}1G2, S1R3B2_BMV_{—}1H5, S1R3C1_CS_{—}1A6, S1R3B2_DP47_{—}1C9, S1R3B2_DP47_{—}1E10, S1R3C1_CS_{—}11310, S1R3A1_BMV_{—}1F3, S1R3B1_BMV_{—}1G11, S1R3A1_BMV_{—}1G4, S1R3B1_BMV_{—}1H11, S1R3A1_CS_{—}1B9, S1R3B1_BMV_{—}1H9, S1R3A1_CS_{—}1B10, S1R3B1_BMV_{—}1C12, S1R3C1_BMV_{—}1H11, S1R3B1_BMV_{—}1A10, S1R3A1_CS_{—}1D11, S1R3C1_DP47_{—}1H1, S1R3A1_CS_{—}1B12, S1R3B1_BMV_{—}1H5, S1R3A1_DP47_{—}1A6, S1R3B1 DP47_{—}1E1 or S1R3B1_BMV_{—}1A1.

18. The binding protein of claim 16, wherein the binding protein comprises:
(a) a VH having the amino acid sequence of any one of SEQ ID NOS: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 65 or 67; or
(b) a VL having the amino acid sequence of any one of SEQ ID NOS: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 63, 64, 66, 68, 69, 70, 71, 72, 73, 74, 75, 76, 77, 78, 79, 80, 81, 82, 83, 84, 85, 86, 87, 88, 89, 90, 91, 92, 93, 94 or 95; or
(c) a VH of (a) and a VL of (b); or
(d) a VH and aVL amino acid sequence of the VH and VL, amino acid sequences, respectively, in any one of S1R2A_CS_{—}1F7, S1R2A_CS_{—}1D11, S1R2C_CS_{—}1D3, S1R2C_CS_{—}1H12, S1R2A_CS_{—}1D3, S1R3B2_BMV_{—}1E1, S1R3C1_CS_{—}1D3, S1R3B2_DP47_{—}1E8, S1R3B2_BMV_{—}1G2, S1R3B2_BMV_{—}1H5, S1R3C1_CS_{—}1A6, S1R3B2_DP47_{—}1C9, S1R3B2_DP47_{—}1E10, S1R3C1_CS_{—}1B10, S1R3A1_BMV_{—}1F3, S1R3B1_BMV_{—}1G11, S1R3A1_BMV_{—}1G4, S1R3B1_BMV_{—}1H11, S1R3A1_CS_{—}1B9, S1R3B1_BMV_{—}1H9, S1R3A1_CS_{—}1B10, S1R3B1_BMV_{—}1C12, S1R3C1_BMV_{—}1H11, S1R3B1_BMV_{—}1A10, S1R3A1_CS_{—}1D11, S1R3C1_DP47_{—}1H1, S1R3A1_CS_{—}1B12, S1R3B1_BMV_{—}1H5, S1R3A1_DP47_{—}1A6, S1R3B1 DP47_{—}1E1 or S1R3B1_BMV_{—}1A1.

19. The binding protein of any one of claims 14-18, which is a SMIP.

20. A SMIP comprising an amino acid sequence that is at least 90% identical to the amino acid sequence of any one of SEQ ID NOS: 159, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231 or 233, excluding the leader sequence.

21. The SMIP of claim 20, comprising an amino acid sequence that is at least 95% identical to the amino acid sequence of any one of SEQ ID NOS: 159, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231 or 233, excluding the leader sequence.

22. The SMIP of claim 20, comprising the amino acid sequence of any one of SEQ ID NOS: 159, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231 or 233, excluding the leader sequence.

23. A nucleic acid molecule encoding the binding protein of any one of claims 14-18 or encoding the SMIP of any one of claims 20-22.

24. A nucleic acid molecule that encodes a binding protein that specifically binds ErbB2, wherein the nucleic acid molecule comprises a nucleotide sequence selected from:
(a) the nucleotide sequence of any one of SEQ ID NOS: 96, 98, 100, 102, 104, 106, 108, 110, 112, 114, 116, 118, 120, 122, 124, 126, 128, 130, 132, 134, 136, 138, 140, 142, 144, 146, 148, 150, 152, 154 or 156; or
(b) the nucleotide sequence of any one of SEQ ID NOS: 97, 99, 101, 103, 105, 107, 109, 111, 113, 115, 117, 119, 121, 123, 125, 127, 129, 131, 133, 135, 137, 139, 141, 143, 145, 147, 149, 151, 153, 155 or 157; or
(c) both the nucleotide sequence of (a) and the nucleotide sequence of (b).

25. The nucleic acid molecule of claim 24, comprising the nucleotide sequence of any one of SEQ ID NOS: 158, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230 or 232.

26. A composition comprising a binding protein of any one of claims 1-18 or the SMIP of any one of claims 20-22.

27. The composition of claim 26, further comprising an additional therapeutic or diagnostic agent.

28. The composition of claim 27 that comprises an additional therapeutic agent, wherein the therapeutic agent is a chemotherapeutic or anti-inflammatory agent.

29. A host cell comprising a nucleic acid molecule of any one of claims 23-25.

30. The host cell of claim 29, selected from the group consisting of an HEK cell, an NS0 cell and a CHO cell.

31. A method for producing a binding molecule of claim 1 or a SMIP of claim 20, comprising the step of culturing the host cell of claim 29 under conditions the permit protein expression.

32. A method for reducing ErbB2-mediated proliferation of a cancer cell comprising the step of administering to a subject or mammal in need thereof an effective amount of a binding protein of claim 2 or a composition of claim 26.

33. A method for reducing tumor growth of an ErbB2-expressing tumor, comprising administering to a subject or mammal in need thereof an effective amount of a binding protein of claim 2 or a composition of claim 26.

34. A method for increasing apoptosis in an ErbB2-expressing tumor, comprising administering to a subject or mammal in need thereof an effective amount of a binding protein of claim 2 or a composition of claim 26.

35. The binding protein of claim 1, which is detectably labeled.

36. A method for detecting an ErbB2 expressing tumor in a subject, comprising administering the binding protein of claim 35.

37. A method for detecting ErbB2 in a sample from a subject comprising the step of contacting the sample with a binding protein of any one of claims 14-19 or a SMIP of claim 20-22 under conditions that permit binding and detecting binding, wherein binding indicates the presence of ErbB2.

38. A method of treating cancer characterized by ErbB2 expression comprising administering to a mammal or subject in need thereof an effective amount of a binding protein of any of claims 1-19.
